SEQUENCE LISTING (126 sequences)

SEQ ID NO: 1 - protein, 232 aa, Homo sapiens
  Amino acid sequence for human invariant chain isoform p35
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 2 - DNA, 699 bp, Homo sapiens
  Nucleotide sequence encoding human invariant chain isoform p35
atgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagagtcactggaactggaggacccgtcttctgggctgggtgtgaccaagcaggatctgggcccagtccccatgtga

SEQ ID NO: 3 - protein, 216 aa, Homo sapiens
  Amino acid sequence for human invariant chain isoform p33
MDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 4 - protein, 75 aa, Mus musculus
MDDQRDLISNHEQLPILGNRPREPERCSRGALYTGVSVLVALLLAGQATTAYFLYQQQGRLDRLTITSQNLQLES

SEQ ID NO: 5 - protein, 296 aa, Homo sapiens
  Amino acid sequence for human invariant chain isoform p43
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKVLTKCQEEVSHIPAVHPGSFRPKCDENGNYLPLQCYGSIGYCWCVFPNGTEVPNTRSRGHHNCSESLELEDPSSGLGVTKQDLGPVPM

SEQ ID NO: 6 - DNA, 891 bp, Homo sapiens
  Nucleotide sequence encoding human invariant chain isoform p43
atgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagtactgaccaagtgccaggaagaggtcagccacatccctgctgtccacccgggttcattcaggcccaagtgcgacgagaacggcaactatctgccactccagtgctatgggagcatcggctactgctggtgtgtcttccccaacggcacggaggtccccaacaccagaagccgcgggcaccataactgcagtgagtcactggaactggaggacccgtcttctgggctgggtgtgaccaagcaggatctgggcccagtccccatgtga

SEQ ID NO: 7 - protein, 280 aa, Homo sapiens
  Amino acid sequence for human invariant chain isoform p41
MDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKVLTKCQEEVSHIPAVHPGSFRPKCDENGNYLPLQCYGSIGYCWCVFPNGTEVPNTRSRGHHNCSESLELEDPSSGLGVTKQDLGPVPM

SEQ ID NO: 8 - DNA, 1185 bp, Gallus gallus
  Nucleotide sequence encoding the OVA antigen
atgggctccatcggcgcagcaagcatggaattttgttttgatgtattcaaggagctcaaagtccaccatgccaatgagaacatcttctactgccccattgccatcatgtcagctctagccatggtatacctgggtgcaaaagacagcaccaggacacagataaataaggttgttcgctttgataaacttccaggattcggagacagtattgaagctcagtgtggcacatctgtaaacgttcactcttcacttagagacatcctcaaccaaatcaccaaaccaaatgatgtttattcgttcagccttgccagtagactttatgctgaagagagatacccaatcctgccagaatacttgcagtgtgtgaaggaactgtatagaggaggcttggaacctatcaactttcaaacagctgcagatcaagccagagagctcatcaattcctgggtagaaagtcagacaaatggaattatcagaaatgtccttcagccaagctccgtggattctcaaactgcaatggttctggttaatgccattgtcttcaaaggactgtgggagaaaacatttaaggatgaagacacacaagcaatgcctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaagcacatcgcaaccaacgccgttctcttctttggcagatgtgtttcccctcatcaccatcaccatcactgataatag

SEQ ID NO: 9 - protein, 160 aa, Homo sapiens
  Amino acid sequence for human invariant chain isoform c
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQSHWNWRTRLLGWV

SEQ ID NO: 10 - DNA, 483 bp, Homo sapiens
  Nucleotide sequence encoding human invariant chain isoform c
atgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagagtcactggaactggaggacccgtcttctgggctgggtgtga

SEQ ID NO: 11 - protein, 215 aa, Homo sapiens
  Amino acid sequence for murine invariant chain p31
MDDQRDLISNHEQLPILGNRPREPERCSRGALYTGVSVLVALLLAGQATTAYFLYQQQGRLDKLTITSQNLQLESLRMKLPKSAKPVSQMRMATPLLMRPMSMDNMLLGPVKNVTKYGNMTQDHVMHLLTRSGPLEYPQLKGTFPENLKHLKNSMDGVNWKIFESWMKQWLLFEMSKNSLEEKKPTEAPPKEPLDMEDLSSGLGVTRQELGQVTL

SEQ ID NO: 12 - DNA, 648 bp, Homo sapiens
  Nucleotide sequence encoding murine invariant chain p31
atggatgaccaacgcgacctcatctctaaccatgaacagttgcccatactgggcaaccgccctagagagccagaaaggtgcagccgtggagctctgtacaccggtgtctctgtcctggtggctctgctcttggctgggcaggccaccactgcttacttcctgtaccagcaacagggccgcctagacaagctgaccatcacctcccagaacctgcaactggagagccttcgcatgaagcttccgaaatctgccaaacctgtgagccagatgcggatggctactcccttgctgatgcgtccaatgtccatggataacatgctccttgggcctgtgaagaacgttaccaagtacggcaacatgacccaggaccatgtgatgcatctgctcacgaggtctggacccctggagtacccgcagctgaaggggaccttcccagagaatctgaagcatcttaagaactccatggatggcgtgaactggaagatcttcgagagctggatgaagcagtggctcttgtttgagatgagcaagaactccctggaggagaagaagcccaccgaggctccacctaaagagccactggacatggaagacctatcttctggcctgggagtgaccaggcaggaactgggtcaagtcaccctgtga

SEQ ID NO: 13 - protein, 279 aa, Homo sapiens
  Amino acid sequence for murine invariant chain p41
MDDQRDLISNHEQLPILGNRPREPERCSRGALYTGVSVLVALLLAGQATTAYFLYQQQGRLDKLTITSQNLQLESLRMKLPKSAKPVSQMRMATPLLMRPMSMDNMLLGPVKNVTKYGNMTQDHVMHLLTRSGPLEYPQLKGTFPENLKHLKNSMDGVNWKIFESWMKQWLLFEMSKNSLEEKKPTEAPPKVLTKCQEEVSHIPAVYPGAFRPKCDENGNYLPLQCHGSTGYCWCVFPNGTEVPHTKSRGRHNCSEPLDMEDLSSGLGVTRQELGQVTL

SEQ ID NO: 14 - DNA, 840 bp, Homo sapiens
  Nucleotide sequence encoding murine invariant chain p41
atggatgaccaacgcgacctcatctctaaccatgaacagttgcccatactgggcaaccgccctagagagccagaaaggtgcagccgtggagctctgtacaccggtgtctctgtcctggtggctctgctcttggctgggcaggccaccactgcttacttcctgtaccagcaacagggccgcctagacaagctgaccatcacctcccagaacctgcaactggagagccttcgcatgaagcttccgaaatctgccaaacctgtgagccagatgcggatggctactcccttgctgatgcgtccaatgtccatggataacatgctccttgggcctgtgaagaacgttaccaagtacggcaacatgacccaggaccatgtgatgcatctgctcacgaggtctggacccctggagtacccgcagctgaaggggaccttcccagagaatctgaagcatcttaagaactccatggatggcgtgaactggaagatcttcgagagctggatgaagcagtggctcttgtttgagatgagcaagaactccctggaggagaagaagcccaccgaggctccacctaaagtactgaccaagtgccaggaagaagtcagccacatccctgccgtctacccgggtgcgttccgtcccaagtgcgacgagaacggtaactatttgccactccagtgccacgggagcactggctactgctggtgtgtgttccccaacggcactgaggttcctcacaccaagagccgcgggcgccataactgcagtgagccactggacatggaagacctatcttctggcctgggagtgaccaggcaggaactgggtcaagtcaccctgtga

SEQ ID NO: 15 - protein, 280 aa, Cavia porcellus
  Amino acid sequence for Cavia porcellus invariant chain (UniProt accession number H0UZ94)
MEDQHDLISNHEQLPMLGQRPGAQDGKCSRGALYTGFSVLVALLLAGQATTAYFLYQQQGRLDKLTVTSQNLQLENLRMKMPKPPKPVSQMRMATPLLMRALPMEVMHKGPVQNATKYGNTTQDYVMHTLLKSDPLKVYPQLQGSFLENLKHLKNTMESLDWKVFESWMHHWLLFEMSRNSPEEKPTEAPPKVLSKCQEEVSHIPAVHPGTFRPQCDENGNYMPLQCHGSTGYCWCVFPNGTEVPHTRSHGHHNCSEPLEAEDLSSGLGVTKQELGQASL

SEQ ID NO: 16 - protein, 329 aa, Heterocephalus glaber
  Amino acid sequence for Heterocephalus glaber invariant chain (UniProt accession number G5C391)
MEDQRDLISNHEQLPMLGQRLGAQDRKCSRGALYTGFSVLVALLLAGQATTAYFLYQQQGRLDKLTVTSQNLQLENLRMKMPQSPKPVSQMRVATPLLMRALPMEGLLQGPMQQNATKYGNMTQDHVMHMLLKSDPLKVYPQLEGSFLDNLKHLKNTMESLDWKVFESWMHHWLLFEMSRNSPGEKPTEAPPKVLSKCQEEVSHIPAVHPGTFRPQCDENGNYMPLQCHGSTGYCWCVFPNGTEVPQTRSRGHHNCSEPLEAEDLSSGLGMTKQELGPAHLAARAKDSSVRKRTCTRCLGLSHRLLCRLLLLGEKGDRLWSLLFLSIAA

SEQ ID NO: 17 - protein, 298 aa, Fukomys damarensis
  Amino acid sequence for Fukomys damarensis invariant chain (UniProt accession number A0A091E9W3)
MEDQRDLISNHEQLPMLGQRPAAQDRKCSRGALYTGFSILVALLLAGQATTAYFLYQQQGRLDKLTVTSQNLQLENLRMKMPKSSKPMTQMRVATPMLMRALPMEGLLQGPMQNATKYGNMTQDHVMHTLLQSDPLKVYPQLTGSFLENLKHLKNTMQSLDWKVFESWMHHWLLFEMSRNSPEKPTEAPPKVLSKCQEEVSHIPAVHPGTFRPQCDENGNYMPLQCHGSTGYCWCVFPNGTEVPHTRSRGHHNCSDPLEAEDLSSGLGVTKQELGPGLCLAKLVISSQGRGSWKNKRG

SEQ ID NO: 18 - protein, 216 aa, Rattus norvegicus
  Amino acid sequence for Rattus norvegicus second isoform invariant chain (UniProt accession number P10247-2)
MDDQRDLISNHEQLPILGQRARAPESNCNRGVLYTSVSVLVALLLAGQATTAYFLYQQQGRLDKLTVTSQNLQLENLRMKLPKSAKPVSPMRMATPLLMRPLSMDNMLQAPVKNVTKYGNMTQDHVMHLLTKSGPVNYPQLKGSFPENLKHLKNSMNGLDWKVFESWMKQWLLFEMSKNSLEEKQPTQTPPKEPLDMEDPSSGLGVTKQDMGQMFL

SEQ ID NO: 19 - protein, 280 aa, Rattus norvegicus
  Amino acid sequence for Rattus norvegicus first isoform invariant chain (UniProt accession number P10247)
MDDQRDLISNHEQLPILGQRARAPESNCNRGVLYTSVSVLVALLLAGQATTAYFLYQQQGRLDKLTVTSQNLQLENLRMKLPKSAKPVSPMRMATPLLMRPLSMDNMLQAPVKNVTKYGNMTQDHVMHLLTKSGPVNYPQLKGSFPENLKHLKNSMNGLDWKVFESWMKQWLLFEMSKNSLEEKQPTQTPPKVLTKCQEEVSHIPDVHPGAFRPKCDENGNYMPLQCHGSTGYCWCVFPNGTEVPHTKSRGRHNCSEPLDMEDPSSGLGVTKQDMGQMFL

SEQ ID NO: 20 - protein, 280 aa, Myotis lucifugus
  Amino acid sequence for Myotis lucifugus invariant chain (UniProt accession number G1QEN4)
MEDQRDLISNHEQLPMLGQRPGAQESKCSRGALYTGFSVLVALLLAGQATTAYFLYQQQGRLDKLTVTSQNLQLENMRMKLPKSAKPVGKMRVATPMLMQALPMDGLLQGPMQNATKYGNTTMDHVMHLLLKADPLKVYPQMKGSFPENLKHLKKTMEGLDWKIFESWMHQWLLFEMSKNSLGEKLTEGSPKVLTKCLEEASRIPAIHPGRFKPQCDENGNYMPLQCFGSIGYCWCVFPNGTEVPHTRSRGRHNCSEPLDMEDLSSGLGVTKQDLVQATM

SEQ ID NO: 21 - protein, 323 aa, Myotis davidii
  Amino acid sequence for Myotis davidii invariant chain (UniProt accession number L5LQM9)
MEDQRDLISNHEQLPMLGQRPGAQESKCSRGALYTGFSVLVALLLAGQATTAYFLYQQQGRLDKLTVTSQNLQLENLRMKLPKSAKPVGKMRVATPMLMQALPMEGLLQGPMQNATKYGNTTMDHVMHLLLKADPLKVYPQMKGSFPENLKHLKKTMEGLDWKIFESWMHQWLLFEMSKNSLGEKLTEGSPKVLTQCLEEASRIPAIHPGRFKPQCDENGNYMPLQCFGSIGYCWCVFPNGTEVPHTRSRGRHNCSEPLDMEDLSSGLGVTKQDLVQAIEDTSTQSALHGHSFLALFRPPNLATYFSPLHALLPPSPTLHLIS

SEQ ID NO: 22 - protein, 295 aa, Myotis brandtii
  Amino acid sequence for Myotis brandtii invariant chain (UniProt accession number S7N2W2)
MLGQRPGAQESKCSRGALYTGFSVLVALLLAGQATTAYFLYQQQGRLDKLTVTSQNLQLENLRMKLPKSAKPVGKMRVATPMLMQALPMEGLLQGPMQNATKYGNTTMDHVMHLLLKADPLKVYPQMKGSFPENLKHLKKTMEGLDWKIFESWMHQWLLFEMSKNSLGEKLTEGSPKVLTKCLEEASRIPAIHPGRFKPQCDENGNYMPLQCFGSIGYCWCVFPNGTEVPHTRSRGRHNCSEPLDMEDLSSGLGVTKQDLVQEITSEQQIRRALLPKPPSISRHTRPKELDHELG

SEQ ID NO: 23 - protein, 299 aa, Pteropus alecto
  Amino acid sequence for Pteropus alecto invariant chain (UniProt accession number L5L1G3)
MEDQRDLISNHEQLPMLGQRPGAPERNCSRGALYTGFSVLVALLLAGQATTAYFLYQQQGRLDKLTVTSQNLQLENLRMKLPKSAKPVSKMRVATPMLMQALPMDGVLQGPMQNATKYGNSTLDHVMHLLLKSDPLKVYPQLKGSFPENLKRLRNTMEGLDWKAFENWMHQWLLFEMSKNSLEEKPKPTQVPTKVLTKCLEEVSRIPAIHPGMFKPKCDENGNYMPLQCYGSIGYCWCVFPNGTEVPHTRSRKRSNCSEPVDMEDLSSGLGVTKQDLSQGKGACRGDAQHGTTLVHSPT

SEQ ID NO: 24 - protein, 232 aa, Pan troglodytes
  Amino acid sequence for Pan troglodytes verus invariant chain (UniProt accession number A5A6L4)
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTEAPPKESLELEDPSSGLGVTKQDLGPVPM

SEQ ID NO: 25 - protein, 232 aa, Pongo abelii
  Amino acid sequence for Pongo abelii invariant chain (UniProt accession number Q5RFJ4)
MHRRRSRSCREDQKPVMDDQRDLISNNEQLPMLGRRPGAPESKCGRGALYTGFSILVTLLLAGQATTAYFLYQQQGRLDKLTVTSQNLQLENLRMKLPKPPKPVSKMRMATPLLMQALPMGALPRGPMQNATKYGNMTEDHVMHLLQNADPLKVYPPLKGSFPENLRHLKNTMETIDWKVFESWMHHWLLFEMSRHSLEQKPTEAPPKESLELEDPSSGLGVTKQDLGPVPM

SEQ ID NO: 26 - protein, 232 aa, Pan troglodytes
  Amino acid sequence for Pan troglodytes invariant chain (UniProt accession number H2QRT2)
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTEAPPKESLELEDPSSGLGVTKQDLGPAPL

SEQ ID NO: 27 - protein, 296 aa, Gorilla gorilla gorilla
  Amino acid sequence for Gorilla gorilla gorilla invariant chain (UniProt accession number G3R7S6)
MHRRSSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTEAPPKVLTKCQEEVSHIPAVHPGSFRPTCDENGNYLPLQCYGSIGYCWCVFPNGTEVPNTRSRGHHNCSESLELEDPSSGLGVTKQDLSPVPM

SEQ ID NO: 28 - protein, 296 aa, Nomascus leucogenys
  Amino acid sequence for Nomascus leucogenys invariant chain (UniProt accession number G1RHB8)
MHRRSSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPRGPMQNATKYGNMTEDHVMHLLQNADPLKVYPPLKGSFPENLRHLKNTMETIDWKVFESWMHHWLLFEMSRHSLEQKPTEAPPKVLTKCQEEVSHIPAVHPGSFRPKCDENGNYLPLQCYGSIGYCWCVFPNGTEVPNTRSRGHHNCSESLELEDPSSGLGVTKQDLGPVPI

SEQ ID NO: 29 - protein, 230 aa, Macaca mulatta
  Amino acid sequence for Macaca mulatta invariant chain (UniProt accession number I0FWR3)
MYRSSRRSCQEDQKPVMDDQRDLISNNEQLPMLGRRPGTPESKCSRGALYTGFSILVTLLLAGQATTAYFLYQQQGRLDKLTVTTQSLQLENLRMKLPKPPKPVSKMRMATPLLMQALPMGAQGPMQNATKYGNMTEDHVMHLLQNADPLKVYPPLKGSFPENLRHLKSTMETLDWKVFESWMHHWLLFEMSKHSLEQKPTEAPPKESLELEDPSSGLGVTKQDLGPVPM

SEQ ID NO: 30 - protein, 346 aa, Macaca fascicularis
  Amino acid sequence for Macaca fascicularis invariant chain (UniProt accession number G7P8P8)
MYRSSRRSCQEDQKPVMDDQRDLISNNEQLPMLGRRPGTPESKCSRGALYTGFSILVTLLLAGQATTAYFLYQQQGRLDKLTVTTQSLQLENLRMKLPKPPKPVSKMRMATPLLMQALPMGAQGPMQNATKYGNMTEDHVMHLLQNADPLKVYPPLKGSFPENLRHLKSTMETLDWKVFESWMHHWLLFEMSKHSLEQKPTEAPPKVLTKCQEEVSRIPAVHPGSFRPKCDENGNYLPLQCYGSTGYCWCVFPNGTEVPNTRSRGHQNCSESLELEDPSSGLGVTKQDLGPVHTQDIILSFMFIHFLPSPPQNHGELDVRGNSLLTFLDLLCLPQLFTMHLQGACP

SEQ ID NO: 31 - protein, 346 aa, Macaca mulatta; identical to SEQ ID NO: 30
  Amino acid sequence for Macaca mulatta invariant chain (UniProt accession number G7MVM5)

SEQ ID NO: 32 - protein, 294 aa, Macaca mulatta
  Amino acid sequence for Macaca mulatta invariant chain (UniProt accession number I0FWR4)
MYRSSRRSCQEDQKPVMDDQRDLISNNEQLPMLGRRPGTPESKCSRGALYTGFSILVTLLLAGQATTAYFLYQQQGRLDKLTVTTQSLQLENLRMKLPKPPKPVSKMRMATPLLMQALPMGAQGPMQNATKYGNMTEDHVMHLLQNADPLKVYPPLKGSFPENLRHLKSTMETLDWKVFESWMHHWLLFEMSKHSLEQKPTEAPPKVLTKCQEEVSRIPAVHPGSFRPKCDENGNYLPLQCYGSTGYCWCVFPNGTEVPNTRSRGHQNCSESLELEDPSSGLGVTKQDLGPVPM

SEQ ID NO: 33 - protein, 270 aa, Macaca mulatta
  Amino acid sequence for Macaca mulatta invariant chain (UniProt accession number F7E9S4)
MYRSSRRSCQEDQKPVMDDQRDLISNNEQLPMLGRRPGTPESKCSHGALYTGFSILVTLLLAGQATTAYFLYQQQGRLDKLTVTTQSLQLENLRMKLPKPPKPVSKMRMATPLLMQALPMGAQGPMQNATKYGNMTEDHVMHLLQNADPLKVYPPLKGSFPENLRHLKSTMETLDWKVFESWMHHWLLFEMSKHSLEQKPTEAPPKVLTKCQEEVSRIPAVHPGSFRPKCDENGNYLPLQCYGSTGYCWCVFPNGTEVPNTRSRGHQNCS

SEQ ID NO: 34 - protein, 294 aa, Papio anubis
  Amino acid sequence for Papio anubis invariant chain (UniProt accession number A0A096MM48)
MYRSSRRSCQEDQKPVMDDQRDLISNNEQLPMLGRRPGTPESKCSRGALYTGFSILVTLLLAGQATTAYFLYQQQGRLDKLTVTTQNLQLENLRMKLPKPPKPVSKMRMATPLLMQALPMGAQGPMQNATKYGNMTEDHVMHLLQNADPLKVYPPLKGSFPENLRHLKSTMETLDWKVFESWMHHWLLFEMSKHSLEQKPTEAPPKVLTKCQEEVSRIPAVHPGSFRPKCDENGNYLPLQCYGSTGYCWCVFPNGTEVPNTRSRGHQNCSESLELEDPSSGLGVTKQDLGPVPM

SEQ ID NO: 35 - protein, 294 aa, Chlorocebus sabaeus
  Amino acid sequence for Chlorocebus sabaeus invariant chain (UniProt accession number A0A0D9RGK4)
MYRSSRRSCQEDQKPVMDDQRDLISNNEQLPMLGRRPGTPESKCSRGALYTGFSILVTLLLAGQATTAYFLYQQQGRLDKLTVTTQNLQLENLRMKLPKPPKPVSKMRMATPLLMQALPMGAQGPMQNATKYGNMTEDHVMHLLQNADPLKVYPPLKGSFPENLRHLKSTMETLDWKVFESWMHHWLLFEMSKHSLEQKPTEAPPKVLTKCQEEVSRIPAVHPGTFRPKCDENGNYLPLQCYGSTGYCWCVFPNGTEVPNTRSRGHQNCSESLELEDPSSGLGVTKQDLGPVPM

SEQ ID NO: 36 - protein, 295 aa, Callithrix jacchus
  Amino acid sequence for Callithrix jacchus invariant chain (UniProt accession number F7ENM4)
VFRRISRNCWEDQKPMDDQRDLISNNEQLPMLGQRPGAPESKCSRGAVYTVFSILVALLLAGQATTAYFLYQQQGRLDKLTVTSQNLQLENLRMKLPKPAKPLSQMRMATPLLMQALPMAGLPQKPMQNATKHGNMTEDHVMHLLLNADPLKVYPPLKGSLSENLKHLKNTMETMDWKVFESWLHHWLLFEMSKHSLEQKPTEAPPKALTKCQEEVSHIPDVHPGSFRPKCDENGNYLPLQCYGSIGYCWCVFPNGTEVPNTRSRGHHNCSESLELEDPSSGLGVTKQDLGPAPL

SEQ ID NO: 37 - protein, 280 aa, Felis catus
  Amino acid sequence for Felis catus invariant chain (UniProt accession number M3VXS2)
MEDQRDLISNHEQLPILGQRPAAPESKCSRGALYTGFSVLVALLLAGQATTAYFLYQQQGRLDKLTVTAQNLQLENLRMKLPKPAKPLNKLRVATPMLMQTMPVRGLLQAPMQNATKYGNMTQDHVMHMLLEGDPLKVYPQLKGNFPENLKHLKNTMGTLDWKVFENWMYQWLLFEMSKNSLEKHPADIPLKVLTKCQEEVSRIPAVHPGTFRPQCDENGNYKPLQCYGSTGYCWCVFPNGTEVPHSRSHGHRNCSESVDVEDLSSGLGMTKPDLGQAPL

SEQ ID NO: 38 - protein, 279 aa, Mustela putorius furo
  Amino acid sequence for Mustela putorius furo invariant chain (UniProt accession number M3YQS4)
MEDQRDLISNHEQLPMLGQRPSAPESKCSRGALYTGFSVLVALLLAGQATTAYFLYQQQGRLDKLTVTSQNLQLENLRMKLPKPPKPLHKMRAATPMLMQALPMPDLLQEPLQNATKYGNMTQDHVMHVLLETDPLKVYPKLKGSFLENLKHLKNTMGPLEWKVFESWMYQWLLFEMSKNSLENKPEVPLKALTQCQEEVSRVPAVHPGTFRPQCDENGNYKPLQCYGSTGYCWCVFPNGTEVPHTRSRGHHNCREPLDMEDLSSGLGMTKQDLGQVAV

SEQ ID NO: 39 - protein, 280 aa, Loxodonta africana
  Amino acid sequence for Loxodonta africana invariant chain (UniProt accession number G3TJE1)
MEDQRDLISNHEQLPILGQRPQAPESKCSRGALYTGFSVLVALLLAGQATTAYFLYQQQGRLDKLTVTAQNLQLENLRMKLPKPAMPMSKMRMATPLLMRALPMEALPHGPMQNATKYGNMPQDYVMHMLLRTDPLKVYPQLKGTLPENLKHLKNTMDGLDWKAFENWMHQWLLFEMSKNSVEEKPTEAPTKALTKCQEEVSRIPAIHPGVYRPKCDENGNYLPLQCYGSTGYCWCVFPNGTEVPHTRSRGHHNCSEPLELEDLSSGVDMTKQGVGEETL

SEQ ID NO: 40 - protein, 280 aa, Loxodonta africana
  Amino acid sequence for Loxodonta africana invariant chain (UniProt accession number G3U7Y6)
MEDQRDLISNHEQLPILGQRPQAPESKCSRGALYTGFSVLVALLLAGQATTAYFLYQQQGRLDKLTVTAQNLQLENLRMKLPKPAMPMSKMRMATPLLMRALPMEALPHGPMQNATKYGNMPQDYVMHMLLRTDPLKVYPQLKGTLPENLKHLKNTMDGLDWKAFENWMHQWLLFEMSKNSVEEKPTEAPTKALTKCQEEVSRIPAIHPGVYRPKCDENGNYLPLQCYGSTGYCWCVFPNGTEVPHTRSRGHHNCSEPLELEDLSSGVDMTKQGVGEGLL

SEQ ID NO: 41 - protein, 214 aa, Sus scrofa
  Amino acid sequence for Sus scrofa invariant chain (UniProt accession number Q764N1)
MEDQRDLISNHEQLPMLGQRPGAPESKCSRGALYTGFSVLVALLLAGQATTAYFLYQQQGRLDKLTVTSQNLQLESLRMKLPKPSKPLSKMRVSAPMLMQALPMEGPEPMRNATKYGNMTQDHVMHLLLKSDPLGVYPKLKGSLPENLKHLKNTMDGVNWKLFENWLRQWLLFEMSKNSLEETPFEVPPKDPLETEDLSSGLGVTKQDLGQVIL

SEQ ID NO: 42 - protein, 340 aa, Camelus ferus
  Amino acid sequence for Camelus ferus invariant chain (UniProt accession number S9XLT6)
MEDQRDLISNHEQLPILGQRPAAPESKCSRGALYTGFSVLMALLLAGQATTAYFLYQQQGRLDKLTITSQNLQLENLRMKLPKPAKPLSQMRMATPMLMQALPMQGPQLMQNATKYGNMTQDHVMHLLLKADPLKVYPQLKGSLPENLKHLKNTMDGMNWKLFENWMHYWLLFEMSKNSQEEQPFEVPTKALTKCQEEVSRIPAIHPGTFRPKCDENGNYMPLQCYGSIGYCWCVFPNGTEVPHTRSRGHHNCSDPLEMEDLSSGLGVTKPDLGQGPTHEALSSSLGPRQMLELPSCPPRVVNDQQGFQTQEAYLPPGVLQTVCSAVFFCEERGMTGSRT

SEQ ID NO: 43 - protein, 268 aa, Bos
  Amino acid sequence for Bos mutus invariant chain (UniProt accession number L8I7V9)
MEDQRDLISNHEQLPMLGQRPGAQESKCSRGALYTGFSVLVALLLAGQATTAYFLYQQQGRLDKLTVTSQNLQLENLRMKLPKPAKPMSQMRMATPMLMRALPMAGPEPMKNATKYGNMTQDHVMHLLLKADPLKVYPQLKGSLPENLKHLKDSMDGLDWKLFESWLHQWLLFEMSKNSLEEKPFEGPPKVLTQCQEEVSRIPAIHPGVFKPNCDENGNYMPLQCYGSIGYCWCVFPNGTEVPHTRSRGHRNCSDPMEMEYPSSGLGV

SEQ ID NO: 44 - protein, 190 aa, Bos taurus
  Amino acid sequence for Bos taurus invariant chain (UniProt accession number Q7JFY1)
ISNHEQLPMLGQRPGAQESKCSRGALYTGFSVLVALLLAGQATTAYFLYQQQGRLDKLTVTSQNLQLENLRMKLPKPAKPMSQMRMATPMLMRALPMAGPEPMKNATKYGNMTQDHVMHLLLKADPLKVYPQLKGSLPENLKHLKDSMDGLDWKLFESWLHQWLLFEMSKNSLEEKPFEGPPKDPMEMEY

SEQ ID NO: 45 - protein, 204 aa, Bos taurus
  Amino acid sequence for Bos taurus invariant chain (UniProt accession number Q29630)
MEDQRDLISNHEQLPMLGQRPGAQESKCSRGALYTGFSVLVALLLAGQATTAYFLYQQQGRLDKLTVTSQNLQLENLRMKLPKPAKPMSQMRMATPMLMRALPMAGPEPMKNATKYGNMTQDHVMHLLLKADPLKVYPQLKGSLPENLKHLKDSMDGLDWKLFESWLHQWLLFEMSKNSLEEKPFEGPPKDPMEMEYPSSGLGV

SEQ ID NO: 46 - protein, 208 aa, Equus caballus
  Amino acid sequence for Equus caballus invariant chain (UniProt accession number F6TGS3)
MEDQRDLISNHEQVPILGQRPAAPERKCSRGALYTGFSVLVALLLAGQATTAYFLFQQQGRLDKLTVTAQNLQLEKLRMKLPKSAKPVSKIRVATPMLMQALPMEGLSHGPMQNATKYGNTTQDHVMHLLLRADPLKVYPQLKGSFQENLKHLKSTMDGLDWKVFENWMHQWLLFEMSRNSLEEKPTQGPTKEPLEIEDLSSGVGMAK

SEQ ID NO: 47 - protein, 208 aa, Equus caballus
  Amino acid sequence for Equus caballus invariant chain (UniProt accession number Q9MXD5)
MEDQRDLISNHEQVPILGQRPAAPERKCSRGALYTGFSVLVALLLAGQATTAYFLFQQQGRPDKLTVTAQNLQLESLRMKLPKSAKPVSKIRVATPMLMQALPMEGLSHGPMQNATKYGNTTQDHVMHLLLRADPLKVYPQLKGSFQENLKHLKSTMDGLDWKVFENWMHQWLLFEMSRNSLEEKPTQGPTKEPLEIEDLSSGVGMAK

SEQ ID NO: 48 - protein, 292 aa, Oryctolagus cuniculus
  Amino acid sequence for Oryctolagus cuniculus invariant chain (UniProt accession number G1SKK3)
FRSQTRKLKTSEARAMDDQRDLISNHEQMPMLGQRPGAQERKCSRGALYTGFSVLVALLLAGQATTAYFLYQQQDRLDKLTVTSQNLQLESLRMKLPKSAKPMSQMRMAAPMMMQALPMENLSQGPVQNVTKYGNTTQDYVMHLLLRSDPLKVYPQLKGSFPENLKQLKGTMDGLNWKVFESWLHQWLLFEMSKNSLEEKPTEAPTKVLSKCLEEASHVPDVHPGRFKPQCDENGNYMPLQCHGSIGYCWCVFPNGTEVPHTRSRGHHNCSEPMEFEYPSSGLDMARPEMGK

SEQ ID NO: 49 - protein, 282 aa, Otolemur garnettii
  Amino acid sequence for Otolemur garnettii invariant chain (UniProt accession number H0WQB3)
MEDQRDLISNHEQTPILSQRAGAPERQCSRGALYTGFSVLVALLLAGQATTAYFLYQQQGRLDKLTVTSQNLQLENLRMKLPKPPKPMSKMRMATPLMMQALPMEGLAQRPVQNATKYGNMTQDHVMHLLLKADPLKVYPQMKGNFPENLKHLKSTMETLDWKVFESWMHQWLLFEMSKNSGEEKPTEAPPKVLTKCQEEFSRVPAIHPGTFRPKCDENGNYMPLQCHGSIGYCWCVFPNGTEVPHTRSRGQHNCSEPQDLEDPSSGLGFTKQEPGIGKGPV

SEQ ID NO: 50 - protein, 375 aa, Tupaia chinensis
  Amino acid sequence for Tupaia chinensis invariant chain (UniProt accession number L9KN01)
MDDQRDLISNHEQVPILGQRPREAESKCGRGALYTGFSVLVALLLAGQATTAYFLYQQQGQLHKLTVTSQNLQLENLRMKLPKPAKPLSKMRMATPLLMRALPMDGLPQGPVQNATKYGNMTQDHVMHLLLKADPLKVYPQLKGSFPENLKHLKSTMETMDWKVFESWMHQWLLFEMSKNSMEEKPTEPPTKALTKCQEEVSRIPAVHPGTFRPKCDENGNYMPLQCHGSIGYCWCVFPNGTEVPHTRSRGHHNCSGPTCLALWDHLDARAALLQEAYLGLVPVALRRSVPLSSSVGEKYERLLKLLWPPEQILGLQGCLRAGQGSASCTLQEGARGSALITQQALWAWVELHPCKVWAVGHEHSPCSGGSDTRK

SEQ ID NO: 51 - protein, 279 aa, Ictidomys tridecemlineatus
  Amino acid sequence for Ictidomys tridecemlineatus invariant chain (UniProt accession number I3MCR9)
MEDQRDLISNHEQLPILGQRPREQERCSRGTLYTGFSVLVALLLAGQATTAYFLYQQQGRLDKLTVTSQNLQLENLRMKLPKPPKPVSQLRMATPLLMQALPMEGLRQGPKQNATKYGNMTQDHVMHLLLKSNPLKVYPQLKGSFPENLKHLKSTMDNLDWKIFENWLHQWLLFEMSKNSLEEKPTEAPTRVLTKCQEEVSHIPAVHPGAFRPKCDENGNYMPLQCHGSIGYCWCVFPNGTEVPHTRSRGRHDCSEPLELEDVSSGLGVTKQDLGQVIM

SEQ ID NO: 52 - protein, 209 aa, Sarcophilus harrisii
  Amino acid sequence for Sarcophilus harrisii invariant chain (UniProt accession number G3X0Q6
MEDQRDLISNHEQQPMLGGSAGGQHRSCNQGAFYTGFSVLVALLIAGQAATVYFVYQQQGRLDKLTVTSQNLQLESLKMKLPKASIPMNKLRLATPMLMRELEPETLPSMDLTKIGNNTKDQVKYLLLQSDPRRSFPELTKSFQENMKKLKNNMETKNWKNFENWMHQWLLFEMSKKPNEENVEKKTEPLQKGLLDEEMFSSGLGFPKQ

SEQ ID NO: 53 - protein, 81 aa, Homo sapiens
  Amino acid sequence for residues 17-97 of human p35 invariant chain
MDDQRDLISNNEQLPMLGRRPGAPESKCSRGALYTGFSILVTLLLAGQATTAYFLYQQQGRLDKLTVTSQNLQLENLRMKL

SEQ ID NO: 54 - protein, 80 aa, Mus musculus
  Amino acid sequence for region of mouse p31 invariant chain corresponding to residues 17-97 of human p35 invariant chain
MDDQRDLISNHEQLPILGNRPREPERCSRGALYTGVSVLVALLLAGQATTAYFLYQQQGRLDKLTITSQNLQLESLRMKL

SEQ ID NO: 55 - protein, 81 aa, Loxodonta africana
  Amino acid sequence for region of Loxodonta africana invariant chain (UniProt accession number G3TJE1) corresponding to residues 17-97 of human p35 invariant chain
MEDQRDLISNHEQLPILGQRPQAPESKCSRGALYTGFSVLVALLLAGQATTAYFLYQQQGRLDKLTVTAQNLQLENLRMKL

SEQ ID NO: 56 - protein, 81 aa, Felis catus
  Amino acid sequence for region of Felis catus invariant chain (UniProt accession number M3VXS2) corresponding to residues 17-97 of human p35 invariant chain
MEDQRDLISNHEQLPILGQRPAAPESKCSRGALYTGFSVLVALLLAGQATTAYFLYQQQGRLDKLTVTAQNLQLENLRMKL

SEQ ID NO: 57 - protein, 81 aa, Equus caballus
  Amino acid sequence for region of Equus caballus invariant chain (UniProt accession number F6TGS3) corresponding to residues 17-97 of human p35 invariant chain
MEDQRDLISNHEQVPILGQRPAAPERKCSRGALYTGFSVLVALLLAGQATTAYFLFQQQGRLDKLTVTAQNLQLEKLRMKL

SEQ ID NO: 58 - protein, 81 aa, Camelus ferus
  Amino acid sequence for region of Camelus ferus invariant chain (UniProt accession number S9XLT6) corresponding to residues 17-97 of human p35 invariant chain
MEDQRDLISNHEQLPILGQRPAAPESKCSRGALYTGFSVLMALLLAGQATTAYFLYQQQGRLDKLTITSQNLQLENLRMKL

SEQ ID NO: 59 - protein, 81 aa, Sus scrofa
  Amino acid sequence for region of Sus scrofa invariant chain (UniProt accession number Q764N1) corresponding to residues 17-97 of human p35 invariant chain
MEDQRDLISNHEQLPMLGQRPGAPESKCSRGALYTGFSVLVALLLAGQATTAYFLYQQQGRLDKLTVTSQNLQLESLRMKL

SEQ ID NO: 60 - protein, 81 aa, Mustela putorius furo
  Amino acid sequence for region of Mustela putorius furo invariant chain (UniProt accession number M3YQS4) corresponding to residues 17-97 of human p35 invariant chain
MEDQRDLISNHEQLPMLGQRPSAPESKCSRGALYTGFSVLVALLLAGQATTAYFLYQQQGRLDKLTVTSQNLQLENLRMKL

SEQ ID NO: 61 - protein, 81 aa, Macaca mulatta
  Amino acid sequence for region of Macaca mulatta invariant chain (UniProt accession number I0FWR3) corresponding to residues 17-97 of human p35 invariant chain
MDDQRDLISNNEQLPMLGRRPGTPESKCSRGALYTGFSILVTLLLAGQATTAYFLYQQQGRLDKLTVTTQSLQLENLRMKL

SEQ ID NO: 62 - protein, 81 aa, Macaca fascicularis; identical to SEQ ID NO: 61
  Amino acid sequence for region of Macaca fascicularis invariant chain (UniProt accession number G7P8P8) corresponding to residues 17-97 of human p35 invariant chain

SEQ ID NO: 63 - protein, 81 aa, Chlorocebus sabaeus
  Amino acid sequence for region of Chlorocebus sabaeus invariant chain (UniProt accession number A0A0D9RGK4) corresponding to residues 17-97 of human p35 invariant chain
MDDQRDLISNNEQLPMLGRRPGTPESKCSRGALYTGFSILVTLLLAGQATTAYFLYQQQGRLDKLTVTTQNLQLENLRMKL

SEQ ID NO: 64 - protein, 81 aa, Papio anubis; identical to SEQ ID NO: 63
  Amino acid sequence for region of Papio anubis invariant chain (UniProt accession number A0A096MM48) corresponding to residues 17-97 of human p35 invariant chain

SEQ ID NO: 65 - protein, 81 aa, Pan troglodytes; identical to SEQ ID NO: 53
  Amino acid sequence for region of Pan troglodytes verus invariant chain (UniProt accession number A5A6L4) corresponding to residues 17-97 of human p35 invariant chain

SEQ ID NO: 66 - protein, 81 aa, Gorilla gorilla gorilla; identical to SEQ ID NO: 53
  Amino acid sequence for region of Gorilla gorilla gorilla invariant chain (UniProt accession number G3R7S6) corresponding to residues 17-97 of human p35 invariant chain

SEQ ID NO: 67 - protein, 81 aa, Nomascus leucogenys; identical to SEQ ID NO: 53
  Amino acid sequence for region of Nomascus leucogenys invariant chain (UniProt accession number G1RHB8) corresponding to residues 17-97 of human p35 invariant chain

SEQ ID NO: 68 - protein, 81 aa, Pongo abelii
  Amino acid sequence for region of Pongo abelii invariant chain (UniProt accession number Q5RFJ4) corresponding to residues 17-97 of human p35 invariant chain
MDDQRDLISNNEQLPMLGRRPGAPESKCGRGALYTGFSILVTLLLAGQATTAYFLYQQQGRLDKLTVTSQNLQLENLRMKL

SEQ ID NO: 69 - protein, 81 aa, Callithrix jacchus
  Amino acid sequence for region of Callithrix jacchus invariant chain (UniProt accession number F7ENE8) corresponding to residues 17-97 of human p35 invariant chain
MDDQRDLISNNEQLPMLGQRPGAPESKCSRGAVYTVFSILVALLLAGQATTAYFLYQQQGRLDKLTVTSQNLQLENLRMKL

SEQ ID NO: 70 - protein, 81 aa, Myotis lucifugus
  Amino acid sequence for region of Myotis lucifugus invariant chain (UniProt accession number G1QEN4) corresponding to residues 17-97 of human p35 invariant chain
MEDQRDLISNHEQLPMLGQRPGAQESKCSRGALYTGFSVLVALLLAGQATTAYFLYQQQGRLDKLTVTSQNLQLENMRMKL

SEQ ID NO: 71 - protein, 81 aa, Myotis davidii
  Amino acid sequence for region of Myotis davidii invariant chain (UniProt accession number L5LQM9) corresponding to residues 17-97 of human p35 invariant chain
MEDQRDLISNHEQLPMLGQRPGAQESKCSRGALYTGFSVLVALLLAGQATTAYFLYQQQGRLDKLTVTSQNLQLENLRMKL

SEQ ID NO: 72 - protein, 81 aa, Bos; identical to SEQ ID NO: 71
  Amino acid sequence for region of Bos mutus invariant chain (UniProt accession number L8I7V9) corresponding to residues 17-97 of human p35 invariant chain

SEQ ID NO: 73 - protein, 81 aa, Bos taurus; identical to SEQ ID NO: 71
  Amino acid sequence for region of Bos taurus invariant chain (UniProt accession number Q29630) corresponding to residues 17-97 of human p35 invariant chain

SEQ ID NO: 74 - protein, 66 aa, Myotis brandtii
  Amino acid sequence for region of Myotis brandtii invariant chain (UniProt accession number S7N2W2) corresponding to residues 17-97 of human p35 invariant chain
MLGQRPGAQESKCSRGALYTGFSVLVALLLAGQATTAYFLYQQQGRLDKLTVTSQNLQLENLRMKL

SEQ ID NO: 75 - protein, 81 aa, Heterocephalus glaber
  Amino acid sequence for region of Heterocephalus glaber invariant chain (UniProt accession number G5C391) corresponding to residues 17-97 of human p35 invariant chain
MEDQRDLISNHEQLPMLGQRLGAQDRKCSRGALYTGFSVLVALLLAGQATTAYFLYQQQGRLDKLTVTSQNLQLENLRMKM

SEQ ID NO: 76 - protein, 81 aa, Fukomys damarensis
  Amino acid sequence for region of Fukomys damarensis invariant chain (UniProt accession number A0A091E9W3) corresponding to residues 17-97 of human p35 invariant chain
MEDQRDLISNHEQLPMLGQRPAAQDRKCSRGALYTGFSILVALLLAGQATTAYFLYQQQGRLDKLTVTSQNLQLENLRMKM

SEQ ID NO: 77 - protein, 81 aa, Cavia porcellus
  Amino acid sequence for region of Cavia porcellus invariant chain (UniProt accession number H0UZ94) corresponding to residues 17-97 of human p35 invariant chain
MEDQHDLISNHEQLPMLGQRPGAQDGKCSRGALYTGFSVLVALLLAGQATTAYFLYQQQGRLDKLTVTSQNLQLENLRMKM

SEQ ID NO: 78 - protein, 81 aa, Oryctolagus cuniculus
  Amino acid sequence for region of Oryctolagus cuniculus invariant chain (UniProt accession number G1SKK3) corresponding to residues 17-97 of human p35 invariant chain
MDDQRDLISNHEQMPMLGQRPGAQERKCSRGALYTGFSVLVALLLAGQATTAYFLYQQQDRLDKLTVTSQNLQLESLRMKL

SEQ ID NO: 79 - protein, 81 aa, Pteropus alecto
  Amino acid sequence for region of Pteropus alecto invariant chain (UniProt accession number L5L1G3) corresponding to residues 17-97 of human p35 invariant chain
MEDQRDLISNHEQLPMLGQRPGAPERNCSRGALYTGFSVLVALLLAGQATTAYFLYQQQGRLDKLTVTSQNLQLENLRMKL

SEQ ID NO: 80 - protein, 81 aa, Rattus norvegicus
  Amino acid sequence for region of Rattus norvegicus second isoform invariant chain (UniProt accession number P10247-2) corresponding to residues 17-97 of human p35 invariant chain
MDDQRDLISNHEQLPILGQRARAPESNCNRGVLYTSVSVLVALLLAGQATTAYFLYQQQGRLDKLTVTSQNLQLENLRMKL

SEQ ID NO: 81 - protein, 81 aa, Tupaia chinensis
  Amino acid sequence for region of Tupaia chinensis invariant chain (UniProt accession number L9KN01) corresponding to residues 17-97 of human p35 invariant chain
MDDQRDLISNHEQVPILGQRPREAESKCGRGALYTGFSVLVALLLAGQATTAYFLYQQQGQLHKLTVTSQNLQLENLRMKL

SEQ ID NO: 82 - protein, 80 aa, Ictidomys tridecemlineatus
  Amino acid sequence for region of Ictidomys tridecemlineatus invariant chain (UniProt accession number I3MCR9) corresponding to residues 17-97 of human p35 invariant chain
MEDQRDLISNHEQLPILGQRPREQERCSRGTLYTGFSVLVALLLAGQATTAYFLYQQQGRLDKLTVTSQNLQLENLRMKL

SEQ ID NO: 83 - protein, 81 aa, Otolemur garnettii
  Amino acid sequence for region of Otolemur garnettii invariant chain (UniProt accession number H0WQB3) corresponding to residues 17-97 of human p35 invariant chain
MEDQRDLISNHEQTPILSQRAGAPERQCSRGALYTGFSVLVALLLAGQATTAYFLYQQQGRLDKLTVTSQNLQLENLRMKL

SEQ ID NO: 84 - protein, 81 aa, Sarcophilus harrisii
  Amino acid sequence for region of Sarcophilus harrisii invariant chain (UniProt accession number G3X0Q6) corresponding to residues 17-97 of human p35 invariant chain
MEDQRDLISNHEQQPMLGGSAGGQHRSCNQGAFYTGFSVLVALLIAGQAATVYFVYQQQGRLDKLTVTSQNLQLESLKMKL

SEQ ID NO: 85 - protein, 26 aa, Homo sapiens
  Amino acid sequence for residues 67-92 of human p35 invariant chain
TAYFLYQQQGRLDKLTVTSQNLQLEN

SEQ ID NO: 86 - protein, 26 aa, Mus musculus
  Amino acid sequence for region of mouse p31 invariant chain corresponding to residues 67-92 of human p35 invariant chain
TAYFLYQQQGRLDKLTITSQNLQLES

SEQ ID NO: 87 - protein, 26 aa, Mustela putorius furo
  Amino acid sequence for region of Mustela putorius furo invariant chain (UniProt accession number M3YQS4) corresponding to residues 67-92 of human p35 invariant chain
TAYFLYQQQGRLDKLTVTSQNLQLEN

SEQ ID NO: 88 - protein, 26 aa, Myotis brandtii
  Amino acid sequence for region of Myotis brandtii invariant chain (UniProt accession number S7N2W2) corresponding to residues 67-92 of human p35 invariant chain
TAYFLYQQQGRLDKLTVTSQNLQLEN

SEQ ID NO: 89 - protein, 26 aa, Pteropus alecto
  Amino acid sequence for region of Pteropus alecto invariant chain (UniProt accession number L5L1G3) corresponding to residues 67-92 of human p35 invariant chain
TAYFLYQQQGRLDKLTVTSQNLQLEN

SEQ ID NO: 90 - protein, 26 aa, Fukomys damarensis
  Amino acid sequence for region of Fukomys damarensis invariant chain (UniProt accession number A0A091E9W3) corresponding to residues 67-92 of human p35 invariant chain
TAYFLYQQQGRLDKLTVTSQNLQLEN

SEQ ID NO: 91 - protein, 26 aa, Ictidomys tridecemlineatus
  Amino acid sequence for region of Ictidomys tridecemlineatus invariant chain (UniProt accession number I3MCR9) corresponding to residues 67-92 of human p35 invariant chain
TAYFLYQQQGRLDKLTVTSQNLQLEN

SEQ ID NO: 92 - protein, 26 aa, Bos
  Amino acid sequence for region of Bos mutus invariant chain (UniProt accession number L8I7V9) corresponding to residues 67-92 of human p35 invariant chain
TAYFLYQQQGRLDKLTVTSQNLQLEN

SEQ ID NO: 93 - protein, 26 aa, Heterocephalus glaber
  Amino acid sequence for region of Heterocephalus glaber invariant chain (UniProt accession number G5C391) corresponding to residues 67-92 of human p35 invariant chain
TAYFLYQQQGRLDKLTVTSQNLQLEN

SEQ ID NO: 94 - protein, 26 aa, Myotis davidii
  Amino acid sequence for region of Myotis davidii invariant chain (UniProt accession number L5LQM9) corresponding to residues 67-92 of human p35 invariant chain
TAYFLYQQQGRLDKLTVTSQNLQLEN

SEQ ID NO: 95 - protein, 26 aa, Tupaia chinensis
  Amino acid sequence for region of Tupaia chinensis invariant chain (UniProt accession number L9KN01) corresponding to residues 67-92 of human p35 invariant chain
TAYFLYQQQGQLHKLTVTSQNLQLEN

SEQ ID NO: 96 - protein, 26 aa, Myotis lucifugus
  Amino acid sequence for region of Myotis lucifugus invariant chain (UniProt accession number G1QEN4) corresponding to residues 67-92 of human p35 invariant chain
TAYFLYQQQGRLDKLTVTSQNLQLEN

SEQ ID NO: 97 - protein, 26 aa, Rattus norvegicus
  Amino acid sequence for region of Rattus norvegicus second isoform invariant chain (UniProt accession number P10247-2) corresponding to residues 67-92 of human p35 invariant chain
TAYFLYQQQGRLDKLTVTSQNLQLEN

SEQ ID NO: 98 - protein, 26 aa, Bos taurus
  Amino acid sequence for region of Bos taurus invariant chain (UniProt accession number Q29630) corresponding to residues 67-92 of human p35 invariant chain
TAYFLYQQQGRLDKLTVTSQNLQLEN

SEQ ID NO: 99 - protein, 26 aa, Otolemur garnettii
  Amino acid sequence for region of Otolemur garnettii invariant chain (UniProt accession number H0WQB3) corresponding to residues 67-92 of human p35 invariant chain
TAYFLYQQQGRLDKLTVTSQNLQLEN

SEQ ID NO: 100 - protein, 26 aa, Cavia porcellus
  Amino acid sequence for region of Cavia porcellus invariant chain (UniProt accession number H0UZ94) corresponding to residues 67-92 of human p35 invariant chain
TAYFLYQQQGRLDKLTVTSQNLQLEN

SEQ ID NO: 101 - protein, 26 aa, Callithrix jacchus
  Amino acid sequence for region of Callithrix jacchus invariant chain (UniProt accession number F7ENE8) corresponding to residues 67-92 of human p35 invariant chain
TAYFLYQQQGRLDKLTVTSQNLQLEN

SEQ ID NO: 102 - protein, 26 aa, Callithrix jacchus
  Amino acid sequence for region of Nomascus leucogenys invariant chain (UniProt accession number G1RHB8) corresponding to residues 67-92 of human p35 invariant chain
TAYFLYQQQGRLDKLTVTSQNLQLEN

SEQ ID NO: 103 - protein, 26 aa, Gorilla gorilla gorilla
  Amino acid sequence for region of Gorilla gorilla gorilla invariant chain (UniProt accession number G3R7S6) corresponding to residues 67-92 of human p35 invariant chain
TAYFLYQQQGRLDKLTVTSQNLQLEN

SEQ ID NO: 104 - protein, 26 aa, Pongo abelii
  Amino acid sequence for region of Pongo abelii invariant chain (UniProt accession number Q5RFJ4) corresponding to residues 67-92 of human p35 invariant chain
TAYFLYQQQGRLDKLTVTSQNLQLEN

SEQ ID NO: 105 - protein, 26 aa, Pan troglodytes
  Amino acid sequence for region of Pan troglodytes verus invariant chain (UniProt accession number A5A6L4) corresponding to residues 67-92 of human p35 invariant chain
TAYFLYQQQGRLDKLTVTSQNLQLEN

SEQ ID NO: 106 - protein, 26 aa, Macaca mulatta
  Amino acid sequence for region of Macaca mulatta invariant chain (UniProt accession number I0FWR3) corresponding to residues 67-92 of human p35 invariant chain
TAYFLYQQQGRLDKLTVTTQSLQLEN

SEQ ID NO: 107 - protein, 26 aa, Macaca fascicularis
  Amino acid sequence for region of Macaca fascicularis invariant chain (UniProt accession number G7P8P8) corresponding to residues 67-92 of human p35 invariant chain
TAYFLYQQQGRLDKLTVTTQSLQLEN

SEQ ID NO: 108 - protein, 26 aa, Chlorocebus sabaeus
  Amino acid sequence for region of Chlorocebus sabaeus invariant chain (UniProt accession number A0A0D9RGK4) corresponding to residues 67-92 of human p35 invariant chain
TAYFLYQQQGRLDKLTVTTQNLQLEN

SEQ ID NO: 109 - protein, 26 aa, Papio anubis
  Amino acid sequence for region of Papio anubis invariant chain (UniProt accession number A0A096MM48) corresponding to residues 67-92 of human p35 invariant chain
TAYFLYQQQGRLDKLTVTTQNLQLEN

SEQ ID NO: 110 - protein, 26 aa, Loxodonta africana
  Amino acid sequence for region of Loxodonta africana invariant chain (UniProt accession number G3TJE1) corresponding to residues 67-92 of human p35 invariant chain
TAYFLYQQQGRLDKLTVTAQNLQLEN

SEQ ID NO: 111 - protein, 26 aa, Felis catus
  Amino acid sequence for region of Felis catus invariant chain (UniProt accession number M3VXS2) corresponding to residues 67-92 of human p35 invariant chain
TAYFLYQQQGRLDKLTVTAQNLQLEN

SEQ ID NO: 112 - protein, 26 aa, Equus caballus
  Amino acid sequence for region of Equus caballus invariant chain (UniProt accession number F6TGS3) corresponding to residues 67-92 of human p35 invariant chain
TAYFLFQQQGRLDKLTVTAQNLQLEK

SEQ ID NO: 113 - protein, 26 aa, Sus scrofa
  Amino acid sequence for region of Sus scrofa invariant chain (UniProt accession number Q764N1) corresponding to residues 67-92 of human p35 invariant chain
TAYFLYQQQGRLDKLTVTSQNLQLES

SEQ ID NO: 114 - protein, 26 aa, Oryctolagus cuniculus
  Amino acid sequence for region of Oryctolagus cuniculus invariant chain (UniProt accession number G1SKK3) corresponding to residues 67-92 of human p35 invariant chain
TAYFLYQQQDRLDKLTVTSQNLQLES

SEQ ID NO: 115 - protein, 26 aa, Sarcophilus harrisii
  Amino acid sequence for region of Sarcophilus harrisii invariant chain (UniProt accession number G3X0Q6) corresponding to residues 67-92 of human p35 invariant chain
TVYFVYQQQGRLDKLTVTSQNLQLES

SEQ ID NO: 116 - protein, 26 aa, Camelus ferus
  Amino acid sequence for region of Camelus ferus invariant chain (UniProt accession number S9XLT6) corresponding to residues 67-92 of human p35 invariant chain
TAYFLYQQQGRLDKLTITSQNLQLEN

SEQ ID NO: 117 - protein, 9 aa, Artificial Sequence
  Amino acid sequence of the ‘res’ linker
SDRYLNRRA

SEQ ID NO: 118 - DNA, 27 bp, Artificial Sequence
  Nucleotide sequence encoding the ‘res’ linker
agcgatcgctatttaaataggcgcgcc

SEQ ID NO: 119 - protein, 9 aa, Artificial Sequence
  Amino acid sequence of the HA tag
YPYDVPDYA

SEQ ID NO: 120 - DNA, 27 bp, Artificial Sequence
  Nucleotide sequence encoding the HA tag
tacccatacgatgttccagattacgct

SEQ ID NO: 121 - protein, 150 aa, Artificial Sequence
  HBV truncated Core protein
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVC

SEQ ID NO: 122 - protein, 185 aa, Artificial Sequence
  HBV truncated Core protein
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 123 - protein, 226 aa, Artificial Sequence
  HBV full-length Surface antigen
MENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 124 - protein, 185 aa, Artificial Sequence
  HBV truncated Core protein
MDIDPYKEFGATVELLSFLPSDFFPSVRDLLDTASALYREALESPEHCSPHHTALRQAILCWGELMTLATWVGNNLEDPASRDLVVNYVNTNMGLKFRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 125 - DNA, 100 bp, Artificial Sequence
  Probe GOI_1
cactgcagcccacaccacaccgcactgaggcaggccatcctgtgctggggagagctgatgaccctcgccacctgggtgggcaacaacctggaggaccccg

SEQ ID NO: 126 - DNA, 100 bp, Artificial Sequence
  Probe GOI_2
caatctgtcctggctaccggtggatgtgcctgaggaggttcatcatcttcctgttcatcctgctcctgtgcctgatcttcctgctggtgctgctggacta

(19) United States
(12) Patent Application Publication
(10) Pub. No.: US 2022/0184158 A1
(43) Pub. Date: Jun. 16, 2022

(54) METHODS OF INDUCING AN IMMUNE RESPONSE

(71) Applicant: GLAXOSMITHKLINE BIOLOGICALS SA, Rixensart (BE)

(72) Inventors: Jean-Francois TOUSSAINT, Rixensart (BE); Vanesa BOL, Rixensart (BE)

(73) Assignee: GLAXOSMITHKLINE BIOLOGICALS SA, Rixensart (BE)

(21) Appl. No.: 17/416,642

(22) PCT Filed: Dec. 20, 2019
(86) PCT No.: PCT/EP2019/086747

§ 371 Date: Jun. 21, 2021

(30) Foreign Application Priority Data
Dec. 21, 2018 (EP) 18215657.0

(51) Int. Cl. A61K 35/768; A61K 39/12; A61K 38/17; A61P 35/00; C12N 7/00

(52) U.S. Cl. CPC A61K 35/768; A61K 39/12; A61K 2039/53; A61P 35/00; C12N 7/00; A61K 38/177

(57) ABSTRACT

There is provided inter alia a method of treating cancer in a mammal, said method comprising the steps of: (i) administering to the mammal a first composition comprising an antigen or comprising a nucleic acid encoding an antigen and (ii) administering to the mammal a second composition comprising an oncolytic virus wherein the oncolytic virus comprises a nucleic acid encoding the antigen.

### CROSS REFERENCE TO RELATED APPLICATIONS

This application is a national stage application, filed under 35 U.S.C. § 371, of International Patent Application No. PCT/EP2019/086747, filed on Dec. 20, 2019, which claims the benefit of European Application No. 18215657.0, filed on Dec. 21, 2018; the disclosures of each are herein incorporated by reference in their entirety.

### SEQUENCE LISTING

The instant application contains an electronically submitted Sequence Listing in ASCII text file format (File name: VB66698_US_SL.txt; Size: 169,277 bytes; and Date of Creation: 18 Feb. 2022) which is hereby incorporated by reference in its entirety.

### FIELD OF THE INVENTION

The present invention relates to methods of inducing an immune response to an antigen in a mammal, in particular for the treatment of cancer.

### BACKGROUND OF THE INVENTION

Tumours may develop in cancer patients because the immune system inadequately detects tumour cells as cells that ought to be destroyed. Tumour cells express autologous tumour antigens in a large proportion of cancer patients. These autologous tumour antigens may elicit a protective anti-tumour immune response. Tumour cells, or tumour cell membranes, have to be internalized by antigen presenting cells in order to induce the development of an anti-tumour cellular immune response. However, the immune system in many cancer patients does not adequately recognise tumour antigens. Improving targeting of the immune system to tumour cells could assist in the treatment of cancer.

Recently, oncolytic viruses (OVs) have been the subject of research efforts for selectively killing tumour cells by lytic replication and thereby reducing tumour size. Oncolytic viruses directly infect and lyse tumour cells, leading to the release of soluble antigens, danger signals and type I interferons, which drive anti-tumour immunity. Useful oncolytic viruses may be naturally non pathogenic or are engineered such that they are no longer pathogenic, i.e. do not significantly replicate in and kill non-tumour cells, but such that they can still enter and kill tumour cells. One exemplary oncolytic virus, herpes simplex virus (HSV), has been suggested to be of use for the oncolytic treatment of cancer. An example of an oncolytic herpesvirus is Talimogene laherparepvec (T-VEC) which has recently been licensed for treating unresectable stage IIIb-IVM1c melanoma. A number of mutations to HSV have been identified which still allow the virus to replicate in culture or in actively dividing cells in vivo (e.g. in tumours), but which prevent significant replication in normal tissue. Strains of vaccinia virus (VACV), Newcastle disease virus (NDV), adenovirus, poliovirus, measles virus and reovirus for example are also currently being investigated for cancer therapy. Although in many studies OVs appeared to be effective antitumour agents with locoregional administration, very few studies have evidenced an enhancement in systemic immune response leading to therapeutic efficacy or characterized immune responses in established distant or metastatic lesions, which presents one of the main limitations for clinical efficacy.

Upon infection of a tumour cell by an oncolytic virus, the tumour cell may become inflamed, thereby potentially becoming more susceptible to targeting by the immune system. However, in some patients the immune response against the tumour may not be optimal, even in the case of an inflamed tumour.

Accordingly, there is a need for improved cancer treatment methods involving oncolytic viruses.

### SUMMARY OF THE INVENTION

In one aspect of the invention there is provided a method of treating cancer in a mammal, said method comprising the steps of:
(i) administering to the mammal a first composition comprising a protein antigen and/or a nucleic acid encoding the antigen and
(ii) administering to the mammal a second composition comprising an oncolytic virus wherein the oncolytic virus comprises a nucleic acid encoding the antigen.

In a further aspect of the invention there is provided a first composition comprising an antigen or comprising a nucleic acid encoding an antigen, said first composition for use in the treatment of cancer in a mammal with a second composition comprising an oncolytic virus wherein the oncolytic virus comprises a nucleic acid encoding the antigen.

Further aspects of the invention will be apparent as follows.

### BRIEF DESCRIPTION OF THE FIGURES

FIG. 1A-1F—Tumour volume (mm³) by day, mouse and group at the left (distal) flank. Mean by group is represented by the bold line

FIG. 2A-2F—Tumour volume (mm³) by day, mouse and group at the right (contra) flank. Mean by group is represented by the bold line

FIG. 3A-3B—Mean of tumour volume (mm³) by group for left (distal) (FIG. 3A) and right (contra) flank (FIG. 3B).

FIG. 4A-4B—Individual AUC of tumour volume for [Day 0-Day 9] and means with their 95% CIs by group. FIG. 4A shows left flank; FIG. 4B shows right flank

FIG. 5A-5B—Mean differences of AUC of tumour volume for [Day 0-Day 9] between groups with 95% CIs for each flank. FIG. 5A shows left flank; FIG. 5B shows right flank

FIG. 6A-6B—Tumour volume of mice at Day 0 just before MVA injection: Mean by group (ChAd_HBV treatment or PBS) for mice selected in the study are presented on the x axis. FIG. 6A shows left flank; FIG. 6B shows right flank.

FIG. 7—Individual percentage of HBV specific CD8+ T cells detected towards the 3 HBV antigens.

FIG. 8—Individual percentage of HBV specific CD4+ T cells detected towards the 3 HBV antigens.

FIG. 9—Percentage of HBs specific CD8+ T cells. Geometric mean ratios and 95% confidence intervals

FIG. 10—Individual percentage of CD3+, CD4+ and CD8+ T cells in tumour cells

FIG. 11A-11B—Geometric mean percentage of CD3+, CD4+ and CD8+ T cells in tumour cells and 95% confidence intervals (FIG. 11A). Geometric mean ratios between groups of CD3+, CD4+ and CD8+ T cells in tumour cells and 95% confidence intervals (FIG. 11B).

FIG. 12—Heatmap of Gene set enrichment analysis for RIGHT flank tumours. Directed global significance statistics measure the extent to which a gene set's genes are up- or down-regulated with the variable. Red denotes gene sets whose genes exhibit extensive over-expression with the covariate, blue denotes gene sets with extensive under-expression.

FIG. 13—Heatmap of Gene set enrichment analysis for LEFT flank tumours. Directed global significance statistics measure the extent to which a gene set's genes are up- or down-regulated with the variable. Red denotes gene sets whose genes exhibit extensive over-expression with the covariate, blue denotes gene sets with extensive under-expression.

### BRIEF DESCRIPTION OF SEQUENCE IDENTIFIERS

SEQ ID No: 1 Amino acid sequence for human invariant chain isoform p35
SEQ ID No: 2 Nucleotide sequence encoding human invariant chain isoform p35
SEQ ID No: 3 Amino acid sequence for human invariant chain isoform p33
SEQ ID No: 4 Polypeptide sequence of mli(1-75)K63R
SEQ ID No: 5 Amino acid sequence for human invariant chain isoform p43
SEQ ID No: 6 Nucleotide sequence encoding human invariant chain isoform p43
SEQ ID No: 7 Amino acid sequence for human invariant chain isoform p41
SEQ ID No: 8 Nucleotide sequence encoding the ovalbumin antigen
SEQ ID No: 9 Amino acid sequence for human invariant chain isoform c
SEQ ID No: 10 Nucleotide sequence encoding human invariant chain isoform c
SEQ ID No: 11 Amino acid sequence for murine invariant chain p31
SEQ ID No: 12 Nucleotide sequence encoding murine invariant chain p31
SEQ ID No: 13 Amino acid sequence for murine invariant chain p41
SEQ ID No: 14 Nucleotide sequence encoding murine invariant chain p41
SEQ ID No: 15 Amino acid sequence for Cavia porcellus invariant chain (UniProt accession number H0UZ94) SEQ ID No: 16 Amino acid sequence for Heterocephalus glaber invariant chain
(UniProt accession number G5C391) SEQ ID No: 17 Amino acid sequence for Fukomys damarensis invariant chain (UniProt accession number A0A091E9W3)
SEQ ID No: 18 Amino acid sequence for Rattus norvegicus second isoform invariant chain (UniProt accession number P10247-2)
SEQ ID No: 19 Amino acid sequence for Rattus norvegicus first isoform invariant chain (UniProt accession number P10247)
SEQ ID No: 20 Amino acid sequence for Myotis lucifugus invariant chain (UniProt accession number G1QEN4)
SEQ ID No: 21 Amino acid sequence for Myotis davidii invariant chain (UniProt accession number L5LQM9)
SEQ ID No: 22 Amino acid sequence for Myotis brandtii invariant chain (UniProt accession number S7N2W2)
SEQ ID No: 23 Amino acid sequence for Pteropus alecto invariant chain (UniProt accession number L5L1G3)
SEQ ID No: 24 Amino acid sequence for Pan troglodytes verus invariant chain (UniProt accession number A5A6L4)
SEQ ID No: 25 Amino acid sequence for Pongo abelii invariant chain (UniProt accession number Q5RFJ4)
SEQ ID No: 26 Amino acid sequence for Pan troglodytes invariant chain (UniProt accession number H2QRT2)
SEQ ID No: 27 Amino acid sequence for Gorilla gorilla gorilla invariant chain (UniProt accession number G3R7S6) SEQ ID No: 28 Amino acid sequence for Nomascus leucogenys invariant chain
(UniProt accession number G1 RHB8)
SEQ ID No: 29 Amino acid sequence for Macaca mulatta invariant chain (UniProt accession number 10FWR3)
SEQ ID No: 30 Amino acid sequence for Macaca fascicularis invariant chain (UniProt accession number G7P8P8)
SEQ ID No: 31 Amino acid sequence for Macaca mulatta invariant chain (UniProt accession number G7MVM5)
SEQ ID No: 32 Amino acid sequence for Macaca mulatta invariant chain (UniProt accession number I0FWR4)
SEQ ID No: 33 Amino acid sequence for Macaca mulatta invariant chain (UniProt accession number F7E9S4)
SEQ ID No: 34 Amino acid sequence for Papio anubis invariant chain (UniProt accession number A0A096MM48)
SEQ ID No: 35 Amino acid sequence for Chlorocebus sabaeus invariant chain (UniProt accession number A0A0D9RGK4)
SEQ ID No: 36 Amino acid sequence for Callithrix jacchus invariant chain (UniProt accession number F7ENM4)
SEQ ID No: 37 Amino acid sequence for Felis catus invariant chain (UniProt accession number M3VXS2)
SEQ ID No: 38 Amino acid sequence for Mustela putorius furo invariant chain (UniProt accession number M3YQS4)
SEQ ID No: 39 Amino acid sequence for Loxodonta africana invariant chain (UniProt accession number G3TJE1)
SEQ ID No: 40 Amino acid sequence for Loxodonta africana invariant chain (UniProt accession number G3U7Y6)
SEQ ID No: 41 Amino acid sequence for Sus scrofa invariant chain (UniProt accession number Q764N1)
SEQ ID No: 42 Amino acid sequence for Camelus ferus invariant chain (UniProt accession number S9XLT6)
SEQ ID No: 43 Amino acid sequence for Bos mutus invariant chain (UniProt accession number L817V9)
SEQ ID No: 44 Amino acid sequence for Bos taurus invariant chain (UniProt accession number Q7JFY1)
SEQ ID No: 45 Amino acid sequence for Bos taurus invariant chain (UniProt accession number Q29630)
SEQ ID No: 46 Amino acid sequence for Equus caballus invariant chain (UniProt accession number F6TGS3)
SEQ ID No: 47 Amino acid sequence for Equus caballus invariant chain (UniProt accession number Q9MXD5)
SEQ ID No: 48 Amino acid sequence for Oryctolagus cuniculus invariant chain (UniProt accession number G1SKK3)
SEQ ID No: 49 Amino acid sequence for Otolemur garnettii invariant chain (UniProt accession number H0WQB3)
SEQ ID No: 50 Amino acid sequence for Tupaia chinensis invariant chain (UniProt accession number L9KN01)
SEQ ID No: 51 Amino acid sequence for Ictidomys tridecemlineatus invariant chain (UniProt accession number I3MCR9)
SEQ ID No: 52 Amino acid sequence for Sarcophilus harrisii invariant chain (UniProt accession number G3X0Q6)
SEQ ID No: 53 Amino acid sequence for residues 17-97 of human p35 invariant chain
SEQ ID No: 54 Amino acid sequence for region of mouse p31 invariant chain corresponding to residues 17-97 of human p35 invariant chain
SEQ ID No: 55 Amino acid sequence for region of Loxodonta africana invariant chain (UniProt accession number G3TJE1) corresponding to residues 17-97 of human p35 invariant chain
SEQ ID No: 56 Amino acid sequence for region of Felis catus invariant chain (UniProt accession number M3VXS2) corresponding to residues 17-97 of human p35 invariant chain
SEQ ID No: 57 Amino acid sequence for region of Equus caballus invariant chain (UniProt accession number F6TGS3) corresponding to residues 17-97 of human p35 invariant chain
SEQ ID No: 58 Amino acid sequence for region of Camelus ferus invariant chain (UniProt accession number S9XLT6) corresponding to residues 17-97 of human p35 invariant chain
SEQ ID No: 59 Amino acid sequence for region of Sus scrofa invariant chain (UniProt accession number Q764N1) corresponding to residues 17-97 of human p35 invariant chain
SEQ ID No: 60 Amino acid sequence for region of Mustela putorius furo invariant chain (UniProt accession number M3YQS4) corresponding to residues 17-97 of human p35 invariant chain
SEQ ID No: 61 Amino acid sequence for region of Macaca mulatta invariant chain (UniProt accession number 10FWR3) corresponding to residues 17-97 of human p35 invariant chain
SEQ ID No: 62 Amino acid sequence for region of Macaca fascicularis invariant chain (UniProt accession number G7P8P8) corresponding to residues 17-97 of human p35 invariant chain
SEQ ID No: 63 Amino acid sequence for region of Chlorocebus sabaeus invariant chain (UniProt accession number A0A0D9RGK4) corresponding to residues 17-97 of human p35 invariant chain
SEQ ID No: 64 Amino acid sequence for region of Papio anubis invariant chain (UniProt accession number A0A096MM48) corresponding to residues 17-97 of human p35 invariant chain
SEQ ID No: 65 Amino acid sequence for region of Pan troglodytes verus invariant chain (UniProt accession number A5A6L4) corresponding to residues 17-97 of human p35 invariant chain
SEQ ID No: 66 Amino acid sequence for region of Gorilla gorilla gorilla invariant chain (UniProt accession number G3R7S6) corresponding to residues 17-97 of human p35 invariant chain
SEQ ID No: 67 Amino acid sequence for region of Nomascus leucogenys invariant chain (UniProt accession number G1 RHB8) corresponding to residues 17-97 of human p35 invariant chain
SEQ ID No: 68 Amino acid sequence for region of Pongo abelii invariant chain (UniProt accession number Q5RFJ4) corresponding to residues 17-97 of human p35 invariant chain
SEQ ID No: 69 Amino acid sequence for region of Callithrix jacchus invariant chain (UniProt accession number F7ENE8) corresponding to residues 17-97 of human p35 invariant chain
SEQ ID No: 70 Amino acid sequence for region of Myotis lucifugus invariant chain (UniProt accession number G1QEN4) corresponding to residues 17-97 of human p35 invariant chain
SEQ ID No: 71 Amino acid sequence for region of Myotis davidii invariant chain (UniProt accession number L5LQM9) corresponding to residues 17-97 of human p35 invariant chain
SEQ ID No: 72 Amino acid sequence for region of Bos mutus invariant chain (UniProt accession number L817V9) corresponding to residues 17-97 of human p35 invariant chain
SEQ ID No: 73 Amino acid sequence for region of Bos taurus invariant chain (UniProt accession number Q29630) corresponding to residues 17-97 of human p35 invariant chain
SEQ ID No: 74 Amino acid sequence for region of Myotis brandtii invariant chain (UniProt accession number S7N2W2) corresponding to residues 17-97 of human p35 invariant chain
SEQ ID No: 75 Amino acid sequence for region of Heterocephalus glaber invariant chain (UniProt accession number G5C391) corresponding to residues 17-97 of human p35 invariant chain
SEQ ID No: 76 Amino acid sequence for region of Fukomys damarensis invariant chain (UniProt accession number A0A091E9W3) corresponding to residues 17-97 of human p35 invariant chain
SEQ ID No: 77 Amino acid sequence for region of Cavia porcellus invariant chain (UniProt accession number H0UZ94) corresponding to residues 17-97 of human p35 invariant chain
SEQ ID No: 78 Amino acid sequence for region of Oryctolagus cuniculus invariant chain (UniProt accession number G1SKK3) corresponding to residues 17-97 of human p35 invariant chain
SEQ ID No: 79 Amino acid sequence for region of Pteropus alecto invariant chain (UniProt accession number L5L1G3) corresponding to residues 17-97 of human p35 invariant chain
SEQ ID No: 80 Amino acid sequence for region of Rattus norvegicus second isoform invariant chain (UniProt accession number P10247-2) corresponding to residues 17-97 of human p35 invariant chain
SEQ ID No: 81 Amino acid sequence for region of Tupaia chinensis invariant chain (UniProt accession number L9KN01) corresponding to residues 17-97 of human p35 invariant chain
SEQ ID No: 82 Amino acid sequence for region of Ictidomys tridecemlineatus invariant chain (UniProt accession number I3MCR9) corresponding to residues 17-97 of human p35 invariant chain
SEQ ID No: 83 Amino acid sequence for region of Otolemur gamettii invariant chain (UniProt accession number H0WQB3) corresponding to residues 17-97 of human p35 invariant chain
SEQ ID No: 84 Amino acid sequence for region of Sarcophilus harrisii invariant chain (UniProt accession number G3X0Q6) corresponding to residues 17-97 of human p35 invariant chain
SEQ ID No: 85 Amino acid sequence for residues 67-92 of human p35 invariant chain
SEQ ID No: 86 Amino acid sequence for region of mouse p31 invariant chain corresponding to residues 67-92 of human p35 invariant chain
SEQ ID No: 87 Amino acid sequence for region of Mustela putorius furo invariant chain (UniProt accession number M3YQS4) corresponding to residues 67-92 of human p35 invariant chain
SEQ ID No: 88 Amino acid sequence for region of Myotis brandtii invariant chain (UniProt accession number S7N2W2) corresponding to residues 67-92 of human p35 invariant chain
SEQ ID No: 89 Amino acid sequence for region of Pteropus alecto invariant chain (UniProt accession number L5L1G3) corresponding to residues 67-92 of human p35 invariant chain
SEQ ID No: 90 Amino acid sequence for region of Fukomys damarensis invariant chain (UniProt accession number A0A091E9W3) corresponding to residues 67-92 of human p35 invariant chain
SEQ ID No: 91 Amino acid sequence for region of Ictidomys tridecemlineatus invariant chain (UniProt accession number I3MCR9) corresponding to residues 67-92 of human p35 invariant chain
SEQ ID No: 92 Amino acid sequence for region of Bos mutus invariant chain (UniProt accession number L817V9) corresponding to residues 67-92 of human p35 invariant chain
SEQ ID No: 93 Amino acid sequence for region of Heterocephalus glaber invariant chain (UniProt accession number G5C391) corresponding to residues 67-92 of human p35 invariant chain
SEQ ID No: 94 Amino acid sequence for region of Myotis davidii invariant chain (UniProt accession number L5LQM9) corresponding to residues 67-92 of human p35 invariant chain
SEQ ID No: 95 Amino acid sequence for region of Tupaia chinensis invariant chain (UniProt accession number L9KN01) corresponding to residues 67-92 of human p35 invariant chain
SEQ ID No: 96 Amino acid sequence for region of Myotis lucifugus invariant chain (UniProt accession number G1QEN4) corresponding to residues 67-92 of human p35 invariant chain
SEQ ID No: 97 Amino acid sequence for region of Rattus norvegicus second isoform invariant chain (UniProt accession number P10247-2) corresponding to residues 67-92 of human p35 invariant chain
SEQ ID No: 98 Amino acid sequence for region of Bos taurus invariant chain (UniProt accession number Q29630) corresponding to residues 67-92 of human p35 invariant chain
SEQ ID No: 99 Amino acid sequence for region of Otolemur gamettii invariant chain (UniProt accession number H0WQB3) corresponding to residues 67-92 of human p35 invariant chain
SEQ ID No: 100 Amino acid sequence for region of Cavia porcellus invariant chain (UniProt accession number H0UZ94) corresponding to residues 67-92 of human p35 invariant chain
SEQ ID No: 101 Amino acid sequence for region of Callithrix jacchus invariant chain (UniProt accession number F7ENE8) corresponding to residues 67-92 of human p35 invariant chain
SEQ ID No: 102 Amino acid sequence for region of Nomascus leucogenys invariant chain (UniProt accession number G1 RHB8) corresponding to residues 67-92 of human p35 invariant chain
SEQ ID No: 103 Amino acid sequence for region of Gorilla gorilla gorilla invariant chain (UniProt accession number G3R7S6) corresponding to residues 67-92 of human p35 invariant chain
SEQ ID No: 104 Amino acid sequence for region of Pongo abelii invariant chain (UniProt accession number Q5RFJ4) corresponding to residues 67-92 of human p35 invariant chain
SEQ ID No: 105 Amino acid sequence for region of Pan troglodytes verus invariant chain (UniProt accession number A5A6L4) corresponding to residues 67-92 of human p35 invariant chain
SEQ ID No: 106 Amino acid sequence for region of Macaca mulatta invariant chain (UniProt accession number 10FWR3) corresponding to residues 67-92 of human p35 invariant chain
SEQ ID No: 107 Amino acid sequence for region of Macaca fascicularis invariant chain (UniProt accession number G7P8P8) corresponding to residues 67-92 of human p35 invariant chain
SEQ ID No: 108 Amino acid sequence for region of Chlorocebus sabaeus invariant chain (UniProt accession number A0A0D9RGK4) corresponding to residues 67-92 of human p35 invariant chain
SEQ ID No: 109 Amino acid sequence for region of Papio anubis invariant chain (UniProt accession number A0A096MM48) corresponding to residues 67-92 of human p35 invariant chain
SEQ ID No: 110 Amino acid sequence for region of Loxodonta africana invariant chain (UniProt accession number G3TJE1) corresponding to residues 67-92 of human p35 invariant chain
SEQ ID No: 111 Amino acid sequence for region of Felis catus invariant chain (UniProt accession number M3VXS2) corresponding to residues 67-92 of human p35 invariant chain
SEQ ID No: 112 Amino acid sequence for region of Equus caballus invariant chain (UniProt accession number F6TGS3) corresponding to residues 67-92 of human p35 invariant chain
SEQ ID No: 113 Amino acid sequence for region of Sus scrofa invariant chain (UniProt accession number Q764N1) corresponding to residues 67-92 of human p35 invariant chain
SEQ ID No: 114 Amino acid sequence for region of Oryctolagus cuniculus invariant chain (UniProt accession number G1SKK3) corresponding to residues 67-92 of human p35 invariant chain
SEQ ID No: 115 Amino acid sequence for region of Sarcophilus harrisii invariant chain (UniProt accession number G3X0Q6) corresponding to residues 67-92 of human p35 invariant chain
SEQ ID No: 116 Amino acid sequence for region of Camelus ferus invariant chain (UniProt accession number S9XLT6) corresponding to residues 67-92 of human p35 invariant chain
SEQ ID No: 117 Amino acid sequence of the ‘res’ linker
SEQ ID No: 118 Nucleotide sequence encoding the ‘res’ linker
SEQ ID No: 119 Amino acid sequence of the HA tag
SEQ ID No: 120 Nucleotide sequence encoding the HA tag
SEQ ID No: 121 Amino acid sequence of HBV truncated Core protein
SEQ ID No: 122 Amino acid sequence of HBV truncated Core protein
SEQ ID No: 123 Amino acid sequence of full-length Surface antigen
SEQ ID No: 124 Amino acid sequence of truncated Core protein
SEQ ID No: 125 DNA probe GOI_1: HBv Core, position 139-238
SEQ ID No: 126 DNA probe GOI_2: HBv Surface, position 200-299

### DETAILED DESCRIPTION OF THE INVENTION

The inventors have provided a method by which tumour cell killing can be enhanced by harnessing a systemic immune response against a dedicated foreign antigen while the same antigen would be carried by a oncolytic virus, thereby labelling the cancer cells for the primed immune system.

### First Composition

Methods of the invention involve the administration of a first composition comprising a protein antigen and/or a nucleic acid encoding the antigen. In particular embodiments, the methods of the invention involve the administration of a first composition comprising a viral vector wherein the viral vector comprises a nucleic acid encoding an antigen. Alternative methods of the invention involve the administration of a first composition comprising an antigen.

Without wishing to be bound by theory, it is believed that administration of the nucleic acid encoding the antigen, or administration of the antigen, serves to elicit an immune response against the antigen. Administration of an oncolytic virus comprising a nucleic acid encoding said antigen in the second composition results in cancer cells then being selectively infected by the oncolytic virus and ‘marks’ them with the antigen for destruction by the immune system. It is also believed that the first composition will elicit a immune response, and in particular CD8+ and/or CD4+ lymphocytes, against the antigen outside the immunosuppressive tumour microenvironment, and that expression of the antigen by the oncolytic virus in the tumour cells will lead to increased tumour infiltration by CD8+ and/or CD4+ lymphocytes primed against the antigen.

Suitably, the antigen used in the invention is a polypeptide and will generally be an isolated polypeptide (i.e. separated from those components with which it may usually be found in nature). For example, a naturally-occurring polypeptide is isolated if it is separated from some or all of the coexisting materials in the natural system. Preferably, such polypeptides are at least about 90% pure, more preferably at least about 95% pure and most preferably at least about 99% pure. Polypeptides may be prepared using any of a variety of well known techniques. Recombinant polypeptides encoded by DNA sequences may be readily prepared from DNA sequences using any of a variety of expression vectors known to those of ordinary skill in the art.

The term “nucleic acid” means a polymeric form of nucleotides of any length, which contain deoxyribonucleotides, ribonucleotides, and/or their analogues. It includes DNA, RNA and DNA/RNA hybrids. It also includes DNA or RNA analogues, such as those containing modified backbones (e.g. peptide nucleic acids (PNAs) or phosphorothioates) or modified bases. Thus nucleic acid includes RNA, mRNA, DNA, cDNA, recombinant nucleic acids, branched nucleic acids, plasmids, etc. Where the nucleic acid takes the form of RNA, it may or may not have a 5′ cap. A nucleic acid, as disclosed herein, can take various forms (e.g. single-stranded, double-stranded, etc.). Nucleic acids may be circular or branched, but will typically be linear. The nucleic acid may, for example, be RNA or DNA.

The nucleic acid may be ‘naked’, i.e. not comprised within a vector. Alternatively, the nucleic acid may be comprised within (for example, be part of) a vector, i.e., part of a construct designed for transduction/transfection of one or more cell types or contained within a delivery vehicle. In one embodiment, the nucleic acid is not a naked DNA.

Vectors may be, for example, expression vectors which are designed to express a nucleotide sequence in a host cell, or viral vectors which are designed to result in the production of a recombinant virus or virus-like particle. Suitably the vector is selected from a viral vector, a virus like particle (VLP), a self-amplifying RNA molecule (SAM) or a bacterial vector.

The RNA may be comprised within a self-amplifying RNA molecule. SAMs have been derived from genomic replicons that lack viral structural proteins and express a heterologous antigen in place of the viral structural proteins. Self-amplifying RNA molecules are known in the art and can be produced by using replication elements derived from, e.g., alphaviruses, and substituting structural viral proteins with a nucleotide sequence encoding a protein of interest. A self-amplifying RNA molecule is typically a plus-strand molecule which can be directly translated after delivery to a cell. This translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of the encoded antigen, or may be transcribed to provide further transcripts with the same sense as the delivered RNA, which are then translated to provide in situ expression of the antigen. The overall result of this sequence of transcriptions is a huge amplification in the number of the introduced replicon RNAs and so the encoded antigen becomes a major polypeptide product of the cells.

One suitable system for achieving self-replication in this manner is to use an alphavirus-based replicon. These replicons are plus-stranded RNAs which lead to the translation of a replicase (or replicase-transcriptase) following their delivery to a cell. The replicase is translated as a polyprotein which auto-cleaves to provide a replication complex which creates genomic-strand copies of the plus-strand delivered RNA. These minus-strand transcripts can themselves be transcribed to give further copies of the plus-stranded parent RNA and also to give a subgenomic transcript which encodes the antigen. Translation of the subgenomic transcript leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used e.g. the attenuated TC83 mutant of VEEV has been used in replicons.

Self-amplifying RNAs contain the basic elements of mRNA, i.e., a cap, 5′UTR, 3′UTR and a poly(A) tail. They additionally comprise a large open reading frame (ORF) that encodes nonstructural viral genes and one or more subgenomic promoter. The nonstructural genes, which include a polymerase, form intracellular RNA replication factories and transcribe the subgenomic RNA at high levels. This mRNA encoding the antigen is amplified in the cell, resulting in high levels of mRNA and antigen expression.

SAMs are suitable vectors according to the invention. Accordingly, in one embodiment the nucleic acid in the first composition is comprised within a SAM.

Bacterial vectors may also be used in the delivery of the nucleic acid. Suitable bacterial vectors include those derived from the Listeria genus such as Listeria monocytogenes.

Most suitably the nucleic acid is comprised within a viral vector. A viral vector is a virus comprising a nucleic acid and which is capable of introducing the nucleic acid into a cell of an organism.

Suitably, the viral vector and the oncolytic virus are not substantially cross-reactive in order to minimize the risk that the administration of the viral vector may impact the potency of the oncolytic virus and vice-versa. Accordingly, in one embodiment, the viral vector is not the same virus as the oncolytic virus of the second composition. Suitably, the viral vector is not an oncolytic virus.

Suitably, the viral vector is immunologically distinct from the oncolytic virus. By ‘immunologically distinct’ it is meant that (a) when administered, the viral vector comprising the nucleic acid encoding the antigen has low cross-reactivity, more suitably substantially no cross-reactivity, with the oncolytic virus when the oncolytic virus does not comprise a nucleic acid encoding the antigen; and (b) when administered, the oncolytic virus comprising the nucleic acid encoding the antigen has low cross-reactivity, more suitably substantially no cross-reactivity, with the viral vector when the viral vector does not comprise a nucleic acid encoding the antigen.

By the term low cross-reactivity is meant that administration of the viral vector does not elicit a notable neutralising antibody response to the oncolytic virus, i.e. not significantly impacting the potency of the oncolytic virus. Desirably, immunisation with the viral vector elicits a neutralising titer which is on average less than 50% of the level arising from immunisation with the oncolytic virus, such as less than 75%, suitably less than 90%. Alternatively, or in addition, immunisation with the oncolytic virus elicits a neutralising titer which is on average less than 50% of the level arising from immunisation with the viral vector, such as less than 75%, suitably less than 90%. Suitably administration of the viral vector induces limited, more suitably substantially no neutralisation (or more suitably no recognition) by the immune system of the oncolytic virus.

Any virus may be used as a viral vector. The virus may be replication competent or replication defective (‘non-replicating’ or ‘replication incompetent’). A replication competent virus is capable of replicating in a mammalian cell, more suitably a human cell, most suitably a human cancer cell. A replication defective virus is incapable of replication in such a cell because for example it has been engineered to comprise at least a functional deletion (or “loss-of-function” mutation).

Suitably the viral vector is selected from adenovirus, retrovirus, lentivirus, adeno-associated virus, herpesvirus, poxvirus (such as vaccinia virus, such as Modified Vaccinia Ankara (MVA)), foamy virus, cytomegalovirus (CMV), Semliki forest virus, Maraba virus, paramyxovirus, flavivirus and arenavirus (such as Lymphocytic choriomeningitis virus (LCMV)).

A particularly suitable viral vector is adenovirus. Adenoviruses have a characteristic morphology with an icosahedral capsid comprising three major proteins, hexon (II), penton base (III) and a knobbed fiber (IV), along with a number of other minor proteins, VI, VIII, IX, IIIa and IVa2. The virus genome is a linear, double-stranded DNA. The virus DNA is intimately associated with the highly basic protein VII and a small peptide pX (formerly termed mu). Another protein, V, is packaged with this DNA-protein complex and provides a structural link to the capsid via protein VI. The virus also contains a virus-encoded protease, which is necessary for processing of some of the structural proteins to produce mature infectious virus.

The adenoviral genome is well characterized. There is general conservation in the overall organization of the adenoviral genome with respect to specific open reading frames being similarly positioned, e.g. the location of the E1A, E1B, E2A, E2B, E3, E4, L1, L2, L3, L4 and L5 genes of each virus. Each extremity of the adenoviral genome comprises a sequence known as an inverted terminal repeat (ITR), which is necessary for viral replication. The virus also comprises a virus-encoded protease, which is necessary for processing some of the structural proteins required to produce infectious virions. The structure of the adenoviral genome is described on the basis of the order in which the viral genes are expressed following host cell transduction. More specifically, the viral genes are referred to as early (E) or late (L) genes according to whether transcription occurs prior to or after onset of DNA replication. In the early phase of transduction, the E1A, E1B, E2A, E2B, E3 and E4 genes of adenovirus are expressed to prepare the host cell for viral replication. During the late phase of infection, expression of the late genes L1-L5, which encode the structural components of the virus particles, is activated.

Suitably the adenovirus is selected from the chimpanzee adenoviruses ChAd3, ChAd63, ChAd19, ChAd155 and ChAd157. WO2005071093 discloses chimpanzee adenoviruses including ChAd3, ChAd19 and ChAd63. WO2016198621 discloses the chimpanzee adenovirus ChAd155. WO2018104911 discloses the chimpanzee adenovirus ChAd157. Such adenoviruses are particularly suitable viral vectors. Further suitable adenoviruses include PanAd1, PanAd2, PanAd3, Pan 5, Pan 6, Pan 7 and Pan 9.

The term replication-competent adenovirus refers to an adenovirus which can replicate in a host cell in the absence of any recombinant helper proteins comprised in the cell. Suitably, a replication-competent adenovirus comprises the following intact or functional essential early genes: E1A, E1B, E2A, E2B, E3 and E4.

The term replication-incompetent or replication-defective adenovirus refers to an adenovirus which is incapable of replication because it has been engineered to comprise at least a functional deletion (or “loss-of-function” mutation), i.e. a deletion or mutation which impairs the function of a gene without removing it entirely, e.g. introduction of artificial stop codons, deletion or mutation of active sites or interaction domains, mutation or deletion of a regulatory sequence of a gene etc, or a complete removal of a gene encoding a gene product that is essential for viral replication, such as one or more of the adenoviral genes selected from E1A, E1B, E2A, E2B, E3 and E4 (such as E3 ORF1, E3 ORF2, E3 ORF3, E3 ORF4, E3 ORF5, E3 ORF6, E3 ORF7, E3 ORF8, E3 ORF9, E4 ORF7, E4 ORF6, E4 ORF4, E4 ORF3, E4 ORF2 and/or E4 ORF1). Particularly suitably E1A, E1B, E3 and/or E4 are deleted. If deleted, the aforementioned deleted gene region will suitably not be considered in the alignment when determining % identity with respect to another sequence.

Other suitable viral vectors include one or more poxviral vectors. Suitably, the poxviral vector belongs to the subfamily chordopoxvirinae, more suitably to a genus in said subfamily selected from the group consisting of orthopox, parapox, yatapox, avipox (suitably canarypox (ALVAC) or fowlpox (FPV)) and molluscipox. Even more suitably, the poxviral vector belongs to the orthopox and is selected from the group consisting of vaccinia virus, NYVAC (derived from the Copenhagen strain of vaccinia), Modified Vaccinia Ankara (MVA), cowpoxvirus and monkeypox virus. Most suitably, the poxviral vector is MVA.

In one embodiment, the first composition comprises a protein antigen and a nucleic acid encoding the antigen. In one embodiment, the protein antigen and the nucleic acid are administered together. In one embodiment, the protein antigen and the nucleic acid are administered separately. In one embodiment, the protein antigen and the nucleic acid are administered simultaneously. In one embodiment, the protein antigen and the nucleic acid are administered sequentially. In one embodiment, the protein antigen is administered together with an adjuvant.

The first composition may comprise an adjuvant. An “adjuvant” as used herein refers to a composition that enhances the immune response to an immunogen. Examples of such adjuvants include but are not limited to inorganic adjuvants (e.g. inorganic metal salts such as aluminium phosphate or aluminium hydroxide), organic adjuvants (e.g. saponins, such as QS21, or squalene), oil-based adjuvants (e.g. Freund's complete adjuvant and Freund's incomplete adjuvant), cytokines (e.g. IL-1β, IL-2, IL-7, IL-12, IL-18, GM-CFS, and INF-γ) particulate adjuvants (e.g. immuno-stimulatory complexes (ISCOMS), liposomes, or biodegradable microspheres), virosomes, bacterial adjuvants (e.g. monophosphoryl lipid A, such as 3-de-O-acylated monophosphoryl lipid A (3D-MPL), or muramyl peptides), synthetic adjuvants (e.g. non-ionic block copolymers, muramyl peptide analogues, or synthetic lipid A), synthetic polynucleotides adjuvants (e.g polyarginine or polylysine) and immunostimulatory oligonucleotides containing unmethylated CpG dinucleotides (“CpG”). Particularly suitable adjuvants are selected from one or more of a saponin, a TLR4 agonist, a TLR7 agonist, a TLR8 agonist, a TLR9 agonist, 3D-MPL, GLA and CRX601.

One especially suitable adjuvant is monophosphoryl lipid A (MPL), in particular 3-de-O-acylated monophosphoryl lipid A (3D-MPL). Chemically it is often supplied as a mixture of 3-de-O-acylated monophosphoryl lipid A with either 4, 5, or 6 acylated chains. It can be purified and prepared by the methods taught in GB 2122204B, which reference also discloses the preparation of diphosphoryl lipid A, and 3-O-deacylated variants thereof. Other purified and synthetic lipopolysaccharides have been described (U.S. Pat. No. 6,005,099 and EP 0 729 473 B1; Hilgers et al., 1986, Int. Arch. Allergy. Immunol., 79(4):392-6; Hilgers et al., 1987, Immunology, 60(1):141-6; and EP 0 549 074 B11).

Saponins are also suitable adjuvants (see Lacaille-Dubois, M and Wagner H, A review of the biological and pharmacological activities of saponins. Phytomedicine vol 2 pp 363-386 (1996)). For example, the saponin Quil A (derived from the bark of the South American tree Quillaja saponaria molina), and fractions thereof, are described in U.S. Pat. No. 5,057,540 and Kensil, Crit. Rev. Ther. Drug Carrier Syst., 1996, 12:1-55; and EP 0 362 279 B1. Purified fractions of Quil A are also known as immunostimulants, such as QS21 and QS17; methods of their production is disclosed in U.S. Pat. No. 5,057,540 and EP 0 362 279 B1. Also described in these references is QS7 (a non-haemolytic fraction of Quil-A). Use of QS21 is further described in Kensil et al. (1991, J. Immunology, 146: 431-437). Combinations of QS21 and polysorbate or cyclodextrin are also known (WO 99/10008). Particulate adjuvant systems comprising fractions of QuilA, such as QS21 and QS7 are described in WO 96/33739 and WO 96/11711.

Another adjuvant is an immunostimulatory oligonucleotide containing unmethylated CpG dinucleotides (“CpG”) (Krieg, Nature 374:546 (1995)). CpG is an abbreviation for cytosine-guanosine dinucleotide motifs present in DNA. CpG is known as an adjuvant when administered by both systemic and mucosal routes (WO 96/02555, EP 468520, Davis et al, J. Immunol, 1998, 160:870-876; McCluskie and Davis, J. Immunol., 1998, 161:4463-6). CpG, when formulated into vaccines, may be administered in free solution together with free antigen (WO 96/02555) or covalently conjugated to an antigen (WO 98/16247), or formulated with a carrier such as aluminium hydroxide (Brazolot-Millan et al., Proc. Natl. Acad. Sci., USA, 1998, 95:15553-8).

Adjuvants such as those described above may be formulated together with carriers, such as liposomes, oil in water emulsions, and/or metallic salts (including aluminum salts such as aluminum hydroxide). For example, 3D-MPL may be formulated with aluminum hydroxide (EP 0 689 454) or oil in water emulsions (WO 95/17210); QS21 may be formulated with cholesterol containing liposomes (WO 96/33739), oil in water emulsions (WO 95/17210) or alum (WO 98/15287); CpG may be formulated with alum (Brazolot-Millan, supra) or with other cationic carriers.

Combinations of adjuvants may be utilized in the present invention, in particular a combination of a monophosphoryl lipid A and a saponin derivative (see, e.g., WO 94/00153; WO 95/17210; WO 96/33739; WO 98/56414; WO 99/12565; WO 99/11241), more particularly the combination of QS21 and 3D-MPL as disclosed in WO 94/00153, or a composition where the QS21 is quenched in cholesterol-containing liposomes (DQ) as disclosed in WO 96/33739. Alternatively, a combination of CpG plus a saponin such as QS21 is an adjuvant suitable for use in the present invention. A potent adjuvant formulation involving QS21, 3D-MPL & tocopherol in an oil in water emulsion is described in WO 95/17210 and is another formulation for use in the present invention. Saponin adjuvants may be formulated in a liposome and combined with an immunostimulatory oligonucleotide. Thus, suitable adjuvant systems include, for example, a combination of monophosphoryl lipid A, preferably 3D-MPL, together with an aluminium salt (e.g. as described in WO00/23105). A further exemplary adjuvant comprises comprises QS21 and/or MPL and/or CpG. QS21 may be quenched in cholesterol-containing liposomes as disclosed in WO 96/33739.

Other suitable adjuvants include alkyl Glucosaminide phosphates (AGPs) such as those disclosed in WO9850399 or U.S. Pat. No. 6,303,347 (processes for preparation of AGPs are also disclosed), or pharmaceutically acceptable salts of AGPs as disclosed in U.S. Pat. No. 6,764,840. Some AGPs are TLR4 agonists, and some are TLR4 antagonists. Both are thought to be useful as adjuvants.

Suitably the adjuvant is in an emulsion formulation, a liposomal formulation or an ISCOM formulation.

It has been found (see WO2007062656, WO2010057501, WO2018172259 and US2016304582 which are incorporated by reference for the purpose of disclosing invariant chain sequences) that the fusion of the invariant chain to an antigen which is comprised by an expression system used for vaccination can increase the immune response against said antigen.

Accordingly, in one embodiment of the invention, the antigen may be co-expressed (at the N-terminus or C-terminus of the antigen) with invariant chain or a functional fragment thereof (‘an invariant chain sequence’). The term “invariant chain”, also known as “Ii” or “CD74” refers to a non-polymorphic type II integral membrane protein. The protein has multiple functions in lymphocyte maturation and adaptive immune responses; in particular li ensures the targeting of newly synthesized MHC II to the endocytic pathway, where the complex can meet antigenic peptides. Additionally, li has been shown to function as an MHC class I chaperone and, by its endosomal targeting sequence, to facilitate stimulation of CD4+, but not CD8+ T-cells directed against covalently linked antigen.

For human invariant chain four different isoforms are known, generally termed p33, p35, p41 and p43. SEQ ID NO: 1 and SEQ ID NO: 2 correspond to the amino acid sequence and the nucleic acid sequence of human invariant chain p35 isoform, respectively. SEQ ID NO: 3 corresponds to the amino acid sequence of human invariant chain p33 isoform. SEQ ID NO: 5 and SEQ ID NO: 6 correspond to the amino acid sequence and the nucleic acid sequence of human invariant chain p43 isoform, respectively. SEQ ID NO: 7 corresponds to the amino acid sequence of human invariant chain p41 isoform. With respect to human p33 and p41 the human p35 and p43 isoforms contain an additional 16 residues at the N-terminus due to alternative initiation of translation. Compared to human p33 and p35 the human p41 and p43 isoforms comprise an additional domain (alternative splicing of exon 6b) inserted in frame in the C-terminal region of the invariant chain. The sequence of an additional human isoform c lacking two exons relative to human p33 and p35 is available in Genbank (Accession BC024272). SEQ ID NO: 9 and SEQ ID NO: 10 correspond to the amino acid sequence and the nucleic acid sequence of human invariant chain c isoform, respectively. Suitably the fragment of invariant chain is derived from human p33, p35, p41, p43 or c isoforms of invariant chain.

If present, the invariant chain must be operably linked to the antigen (i.e. the nucleotide sequence encoding the antigen). An operative link either refers to a direct link or to a sequence of amino acid residues or nucleotides that bind together the of invariant chain and the antigenic sequence or the encoded of invariant chain and antigenic sequence, such that on administration of the fusion protein, the invariant chain increases the immunological response to the antigenic sequence substantially to the same extent as that of the invariant chain directly linked to the antigenic sequence. A direct link is when the 3′ end of the first polynucleotide is directly adjacent to the 5′ end of the second sequence with no intervening nucleic acids. Alternatively, the ORFs may be indirectly linked such that there are intervening nucleic acids. For example, the intervening nucleic acids may be noncoding or may encode an amino acid sequence, for example a peptide linker. Operatively-linked nucleic acids may encode polypeptides that are directly linked, i.e., the carboxy-terminus (“C-terminus”) of one encoded polypeptide is directly adjacent to the amino-terminus (“N-terminus”) of a second encoded polypeptide. Alternatively, operatively-linked nucleic acids may encode indirectly linked polypeptides such that there are intervening amino acids between the encoded polypeptides. Such intervening amino acids are referred to herein as a peptide sequence or linker.

In one embodiment the invariant chain is directly linked to the antigenic sequence. In an alternative embodiment, the invariant chain is indirectly linked to the antigenic sequence. Suitably the invariant chain is indirectly linked to the antigenic sequence by a peptide sequence. Suitably the peptide sequence comprises or more suitably consists of glycine and serine, more suitably the peptide sequence comprises or more suitably consists of the sequence GlySer. Alternatively, the peptide sequence comprises or consists of the ‘AscI’ linker, which is a linker having the polypeptide sequence ArgArgAla, encoded by polynucleotide sequence AGGCGCGCC. Alternatively, the peptide sequence comprises or more suitably consists of the ‘res’ linker, which is a linker having the polypeptide sequence SerAspArgTyrLeuAsnArgArgAla (SEQ ID NO: 117), encoded by polynucleotide sequence AGCGATCGCTATTTAAATAGGCGCGCC (SEQ ID NO: 118). Alternatively, the peptide sequence comprises or more suitably consists of the human influenza hemagglutinin (HA) tag (polypeptide SEQ ID NO: 119, polynucleotide SEQ ID NO: 120).

A functional fragment of invariant chain is a portion of a full length invariant chain sequence which, when co-expressed with and operatively linked to antigen, enhances the immunogenic properties of the antigen beyond that which would have bene achieved without co-expression of the fragment of invariant chain. The enhancement in immunogenic properties may be increases in CD4+ and/or CD8+ and/or antibody responses. All ‘functional fragments of invariant chain’ referred to herein are functional in this respect.

Suitable functional fragments of invariant chain include those recited in WO2018037045. Suitably a functional fragment of invariant chain is a fragment of at least 10, more suitably 20, more suitably 30, more suitably 40, more suitably 50, more suitably 80, more suitably 150 amino acids of invariant chain which substantially maintains the properties described above.

Alternatively, or in addition, a functional fragment of invariant chain is a polypeptide sequence sharing suitably at least 50% identity, more suitably 70% identity, more suitably 90% identity, more suitably 95% identity with a full length invariant chain sequence or a fragment of invariant chain sequence and substantially maintains the properties described above.

Suitably the functional fragment of invariant chain comprises or consists of a portion of residues 17-97 of SEQ ID NO: 1, wherein the portion comprises at least 5 contiguous residues from residues 77-92 of SEQ ID NO: 1 (human invariant chain p35 isoform), or the corresponding sequence from the invariant chain protein derived from another human invariant chain isoform or the invariant chain protein derived from an organism other than a human, such as those recited in SEQ ID NOs: 5, 9, 11, 13 and 15-52.

More suitably the functional fragment of invariant chain comprises or consists of residues 67-76, 68-77, 69-78, 70-79, 71-80, 72-81, 73-82, 74-83, 75-84, 76-85, 77-86, 78-87, 79-88, 80-89, 81-90, 82-91 or 83-92 of SEQ ID NO: 1; 67-81, 68-82, 69-83, 70-84, 71-85, 72-86, 73-87, 74-88, 75-89, 76-90, 77-91 or 78-92 of SEQ ID NO: 1; or 67-86, 68-87, 69-88, 70-89, 71-90, 72-91 or 73-92 of SEQ ID NO: 1.

Suitably the fragment of invariant chain refers to a truncated version of an invariant chain derived from an animal, such as a vertebrate, such as a fish, bird or mammal. Suitable truncated versions of invariant chain are provided in SEQ ID NOs: 4 and 53-116. More suitably the fragment of invariant chain refers to a truncated version of an invariant chain derived from a mammal. More suitably the fragment of invariant chain refers to a truncated version of an invariant chain derived from a mammal selected from the list consisting of a chicken, cow, dog, mouse, rat, non-human primate or human. More suitably the fragment of invariant chain refers to a truncated version of an invariant chain derived from a human or mouse. More suitably the fragment of invariant chain refers to a truncated version of an invariant chain derived from a human.

Different invariant chain sequences from various species are provided in SEQ ID NOs: 1, 5, 9, 11, 13 and 15-52. These invariant chain sequences or fragments or variants thereof, are all suitable for use in the present invention.

For murine invariant chain only two isoforms (p31 and p41) are known corresponding to the human invariant chain isoforms p33 and p41, respectively. SEQ ID NO: 11 and SEQ ID NO: 12 correspond to the amino acid sequence and the nucleic acid sequence of murine invariant chain p31 isoform, respectively. SEQ ID NO: 13 and SEQ ID NO: 14 correspond to the amino acid sequence and the nucleic acid sequence of murine invariant chain p41 isoform, respectively. Suitably the fragment of invariant chain is derived from mouse p31 or p41 isoforms of invariant chain. A particularly suitably fragment of invariant chain derived from mouse invariant chain is provided in SEQ ID NO: 4 (mli(1-75)K63R).

Suitably the functional fragment of invariant chain comprises or consists of a portion of residues 1-80 of SEQ ID NO: 11, wherein the portion comprises at least 10 contiguous residues from residues 50-75 of SEQ ID NO: 1.

In particular, the portion above may comprise or more suitably consist of residues 53-75, 55-75, 56-75, 60-75, 62-75 or 68-75 of SEQ ID NO: 11. Alternatively, the portion above may comprise or more suitably consist of residues 50-73, 50-70 or 50-65 of SEQ ID NO: 11. More suitably, the portion above may comprise or more suitably consist of residues 55-75 or 60-75 of SEQ ID NO: 11.

All references above to invariant chain are also equally applicable to functional fragments of invariant chain. As used herein ‘an invariant chain sequence’ refers to either the full length sequence of invariant chain, or a functional fragment or variant of invariant chain.

In one embodiment of the invention, the antigen may be co-expressed (at the N-terminus or C-terminus of the antigen) with flagellin or a functional fragment thereof. Alternatively, the antigen may be delivered in co-formulation with flagellin.

Flagellin represents a pathogen associated molecular pattern (PAMP) that can interact with the TLR5 receptor as well as with at least two cytosolic PRR receptors. Fusion of flagellin to an antigen is a way to potentially make the antigen more immunologically potent and therefore effective. Without wishing to be bound by theory, it is thought that flagellin works by binding Toll-like receptor 5 (TLR5) which is present on cells of the innate immune system. TLRs recognize certain ‘patterns’ that are conserved in flagellin. Binding of flagellin to the TLR5 receptor triggers a series of innate and adaptive immune responses that are necessary for orchestration of an effective immune response.

### Second Composition

The methods of the invention involve the administration of an oncolytic virus (OV). The oncolytic virus may destroy cancer cells by mechanisms such as apoptosis, necroptosis and immunologic cell death, or render the infected cancer cells immunogenic by eliciting over-expression of MHC, by activating pattern recognition receptors or other mechanisms of pathogen sensing and/or by triggering release of cytokines such as interferon type I. However, without wishing to be bound by theory, the invention provides a further key mechanism by which the oncolytic virus facilitates destruction of cancer cells by the immune system. It is believed that the oncolytic virus comprising an antigen infects cancer cells and ‘marks’ said cells with the antigen for destruction by the immune system, and in particular by tumour infiltrating lymphocytes (TILs). Importantly (and in addition to the direct effects of the oncolytic virus on the cancer cells), the destruction of these ‘marked’ cancer cells is facilitated or enhanced by the administration of the first composition which comprises a nucleic acid encoding the antigen and/or the polypeptide antigen, which serves to generate an immune response against the antigen.

Suitably, administration of the oncolytic virus induces limited, more suitably substantially no neutralisation (or more suitably no recognition) by the immune system of the nucleic acid encoding the antigen in the first composition. More particularly, if the nucleic acid encoding the antigen in the first composition is delivered via a viral vector, then the oncolytic virus induces limited, more suitably substantially no neutralisation (or more suitably no recognition) by the immune system of the viral vector.

The oncolytic virus is a virus that infects and/or replicates within cancer cells. Suitably, the oncolytic virus substantially infects selectively and/or replicates within cancer cells. The oncolytic virus comprises an antigen and suitably the oncolytic virus ‘marks’ cancer cells with the antigen allowing them to be more readily distinguished from non-cancer cells, particularly due to a response being mounted to the antigen by administration of the first composition comprising the antigen. In some cases, the oncolytic virus may lyse the cancer cells. In principle any virus capable of infection of and/or replication in cancer cells including cells of tumours, neoplasms, carcinomas, sarcomas, and the like may be utilized as oncolytic virus in the invention. In a preferred embodiment, the oncolytic virus selectively infects and/or replicates in cancer cells.

A number of viruses including adenovirus, reovirus, measles, Newcastle disease virus, poliovirus, paramyxovirus, poxvirus, picornavirus, herpesvirus, vaccinia virus (such as MVA), retrovirus, orthomyxovirus and arenavirus (such as LCMV) have now been identified as oncolytic agents. Many oncolytic viruses may be further engineered for tumour selectivity, productivity, safety etc., although there are naturally occurring examples.

The oncolytic virus is suitably non-replicating or alternatively, replication competent. The oncolytic virus may substantially selectively infect only cancer cells. Selective infection in cancer cells suitably means that the virus replicates at least 1×10³ times, 1×10⁴ times, 1×10⁵ times, 1×10⁶ times, or more, more efficiently in at least three cell lines established from different tumours compared to cells from at least three different non-tumorigenic tissues.

Suitably the oncolytic virus preferentially infects cancer cells. Suitably the oncolytic virus substantially infects only cancer cells, more suitably the oncolytic virus elicits expression of the antigen within the cancer cells and/or elicits presentation of the antigen on the surface of the cancer cells, more suitably the oncolytic virus replicates within the cancer cells and more suitably the oncolytic virus induces immunogenic cell death of the cancer cells or kills the cancer cells.

Suitably the oncolytic virus does not induce immunogenic cell death of non-cancer cells or kill non-cancer cells, more suitably the oncolytic virus does not replicate within non-cancer cells, more suitably the oncolytic virus does not elicit expression of the antigen within the cancer cells and/or elicit presentation of the antigen on the surface of the cancer cells, more suitably the oncolytic virus does not substantially infect non-cancer cells.

In one embodiment, the oncolytic virus has been engineered to be oncolytic. In an alternative embodiment, the virus is naturally oncolytic. In one embodiment, the virus only infects and/or replicates within and/or lyses cancer cells. In one embodiment the virus does not infect and/or replicate within and/or lyse non-cancer cells.

Suitably the oncolytic virus is an enveloped virus derived from the virus families herpesviridae, poxviridae, rhabdoviridae, or paramyxoviridae.

Suitable oncolytic viruses include adenovirus, adeno-associated virus, influenza virus, reovirus, vesicular stomatitis virus (VSV), Newcastle disease virus (NDV), vaccinia virus (in particular MVA), poliovirus, measles virus, mumps virus, sindbis virus (SrN), paramyxovirus, poxvirus, picornavirus, herpesvirus, retrovirus, orthomyxovirus, arenavirus and sendai virus (SV). Further exemplary oncolytic viruses are recited in Kaufman et al 2015 Nature Reviews Drug Discovery 14:642-662.

Oncolytic viruses may additionally encode a heterologous gene (or genes) that encodes for a protein, which has additional anti-tumour properties. The ideal oncolytic virus efficiently kills a clinically relevant fraction of the patient's cancer cells by direct cytolysis with a minimal destruction of non-neoplastic tissue.

Suitably the oncolytic virus comprises additional molecules which increase its immune activation potential, such as cytokines, immunostimulants and pro-apoptotic molecules. Ideally the additional molecules modulate a pathway other than those already exploited by the oncolytic virus. Suitably the oncolytic virus comprises a nucleic acid encoding an immune system signalling molecule which is expressed only in tumour cells. Expression only in tumour cells may be achieved by incorporating RNA destabilising elements, miRNA-targets, tissue specific promoter or transcription factors, or by expression of a ligand (such as a monoclonal antibody, Fab or small molecule) which binds to a molecule preferentially expressed at the surface of tumour cells.

The oncolytic virus may further comprise immune modulators to increase tumour-specificity, as described in Ahmed et al 2003 Nat Biotechnol 21(7):771-777 and Baertsch et al 2014 Cancer Gene Ther 21(9):373-380. Particular immune modulators are PD-1 or other checkpoint antibodies.

Oncolytic viruses are particularly suitable (from a safety perspective) if they naturally are not pathogenic (e.g. naturally do not infect humans) or only cause mild disease in humans (e.g. adenoviruses cause flu-like symptoms). Oncolytic viruses that have been used successfully in approved vaccines (e.g. small pox vaccine) are also preferred for this reason. If an oncolytic virus is pathogenic in humans and is linked to significant disease (e.g. neurotoxicity associated with some herpes virus strains) then it is preferred to make multiple deletions or mutations in the viral genome to render them specific for cancer cells and reduce the risk that a single genetic recombination event with an endogenous virus leads to a fully pathogenic strain.

In one embodiment, the oncolytic virus is administered in an effective amount to infect at least one cancer cell in the individual.

The second composition may comprise an adjuvant. Examples of such adjuvants include but are not limited to inorganic adjuvants (e.g. inorganic metal salts such as aluminium phosphate or aluminium hydroxide), organic adjuvants (e.g. saponins, such as QS21, or squalene), oil-based adjuvants (e.g. Freund's complete adjuvant and Freund's incomplete adjuvant), cytokines (e.g. IL-1β, IL-2, IL-7, IL-12, IL-18, GM-CFS, and INF-γ) particulate adjuvants (e.g. immuno-stimulatory complexes (ISCOMS), liposomes, or biodegradable microspheres), virosomes, bacterial adjuvants (e.g. monophosphoryl lipid A, such as 3-de-O-acylated monophosphoryl lipid A (3D-MPL), or muramyl peptides), synthetic adjuvants (e.g. non-ionic block copolymers, muramyl peptide analogues, or synthetic lipid A), synthetic polynucleotides adjuvants (e.g polyarginine or polylysine) and immunostimulatory oligonucleotides containing unmethylated CpG dinucleotides (“CpG”). Particularly suitable adjuvants are selected from one or more of a saponin, a TLR4 agonist, a TLR7 agonist, a TLR8 agonist, a TLR9 agonist, a STING agonist, 3D-MPL, GLA and CRX601.

One especially suitable adjuvant is monophosphoryl lipid A (MPL), in particular 3-de-O-acylated monophosphoryl lipid A (3D-MPL). Chemically it is often supplied as a mixture of 3-de-O-acylated monophosphoryl lipid A with either 4, 5, or 6 acylated chains. It can be purified and prepared by the methods taught in GB 2122204B, which reference also discloses the preparation of diphosphoryl lipid A, and 3-O-deacylated variants thereof. Other purified and synthetic lipopolysaccharides have been described (U.S. Pat. No. 6,005,099 and EP 0 729 473 B1; Hilgers et al., 1986, Int. Arch. Allergy. Immunol., 79(4):392-6; Hilgers et al., 1987, Immunology, 60(1):141-6; and EP 0 549 074 B11).

Saponins are also suitable adjuvants (see Lacaille-Dubois, M and Wagner H, A review of the biological and pharmacological activities of saponins. Phytomedicine vol 2 pp 363-386 (1996)). For example, the saponin Quil A (derived from the bark of the South American tree Quillaja saponaria molina), and fractions thereof, are described in U.S. Pat. No. 5,057,540 and Kensil, Crit. Rev. Ther. Drug Carrier Syst., 1996, 12:1-55; and EP 0 362 279 B1. Purified fractions of Quil A are also known as immunostimulants, such as QS21 and QS17; methods of their production is disclosed in U.S. Pat. No. 5,057,540 and EP 0 362 279 B1. Also described in these references is QS7 (a non-haemolytic fraction of Quil-A). Use of QS21 is further described in Kensil et al. (1991, J. Immunology, 146: 431-437). Combinations of QS21 and polysorbate or cyclodextrin are also known (WO 99/10008). Particulate adjuvant systems comprising fractions of QuilA, such as QS21 and QS7 are described in WO 96/33739 and WO 96/11711.

Another adjuvant is an immunostimulatory oligonucleotide containing unmethylated CpG dinucleotides (“CpG”) (Krieg, Nature 374:546 (1995)). CpG is an abbreviation for cytosine-guanosine dinucleotide motifs present in DNA. CpG is known as an adjuvant when administered by both systemic and mucosal routes (WO 96/02555, EP 468520, Davis et al, J. Immunol, 1998, 160:870-876; McCluskie and Davis, J. Immunol., 1998, 161:4463-6). CpG, when formulated into vaccines, may be administered in free solution together with free antigen (WO 96/02555) or covalently conjugated to an antigen (WO 98/16247), or formulated with a carrier such as aluminium hydroxide (Brazolot-Millan et al., Proc. Natl. Acad. Sci., USA, 1998, 95:15553-8).

Adjuvants such as those described above may be formulated together with carriers, such as liposomes, oil in water emulsions, and/or metallic salts (including aluminum salts such as aluminum hydroxide). For example, 3D-MPL may be formulated with aluminum hydroxide (EP 0 689 454) or oil in water emulsions (WO 95/17210); QS21 may be formulated with cholesterol containing liposomes (WO 96/33739), oil in water emulsions (WO 95/17210) or alum (WO 98/15287); CpG may be formulated with alum (Brazolot-Millan, supra) or with other cationic carriers.

Combinations of adjuvants may be utilized in the present invention, in particular a combination of a monophosphoryl lipid A and a saponin derivative (see, e.g., WO 94/00153; WO 95/17210; WO 96/33739; WO 98/56414; WO 99/12565; WO 99/11241), more particularly the combination of QS21 and 3D-MPL as disclosed in WO 94/00153, or a composition where the QS21 is quenched in cholesterol-containing liposomes (DQ) as disclosed in WO 96/33739. Alternatively, a combination of CpG plus a saponin such as QS21 is an adjuvant suitable for use in the present invention. A potent adjuvant formulation involving QS21, 3D-MPL & tocopherol in an oil in water emulsion is described in WO 95/17210 and is another formulation for use in the present invention. Saponin adjuvants may be formulated in a liposome and combined with an immunostimulatory oligonucleotide. Thus, suitable adjuvant systems include, for example, a combination of monophosphoryl lipid A, preferably 3D-MPL, together with an aluminium salt (e.g. as described in WO00/23105). A further exemplary adjuvant comprises comprises QS21 and/or MPL and/or CpG. QS21 may be quenched in cholesterol-containing liposomes as disclosed in WO 96/33739.

Other suitable adjuvants include alkyl Glucosaminide phosphates (AGPs) such as those disclosed in WO9850399 or U.S. Pat. No. 6,303,347 (processes for preparation of AGPs are also disclosed), or pharmaceutically acceptable salts of AGPs as disclosed in U.S. Pat. No. 6,764,840. Some AGPs are TLR4 agonists, and some are TLR4 antagonists. Both are thought to be useful as adjuvants.

Suitably the adjuvant is in an emulsion formulation, a liposomal formulation or an ISCOM formulation.

In one embodiment of the invention, the antigen may be co-expressed (at the N-terminus or C-terminus of the antigen) with invariant chain or a functional fragment thereof (‘an invariant chain sequence’).

If present, the invariant chain must be operably linked to the antigen (i.e. the nucleotide sequence encoding the antigen). An operative link either refers to a direct link or to a sequence of amino acid residues or nucleotides that bind together the of invariant chain and the antigenic sequence or the encoded of invariant chain and antigenic sequence, such that on administration of the fusion protein, the invariant chain increases the immunological response to the antigenic sequence substantially to the same extent as that of the invariant chain directly linked to the antigenic sequence. A direct link is when the 3′ end of the first polynucleotide is directly adjacent to the 5′ end of the second sequence with no intervening nucleic acids. Alternatively, the ORFs may be indirectly linked such that there are intervening nucleic acids. For example, the intervening nucleic acids may be noncoding or may encode an amino acid sequence, for example a peptide linker. Operatively-linked nucleic acids may encode polypeptides that are directly linked, i.e., the carboxy-terminus (“C-terminus”) of one encoded polypeptide is directly adjacent to the amino-terminus (“N-terminus”) of a second encoded polypeptide. Alternatively, operatively-linked nucleic acids may encode indirectly linked polypeptides such that there are intervening amino acids between the encoded polypeptides. Such intervening amino acids are referred to herein as a peptide sequence or linker.

In one embodiment the invariant chain is directly linked to the antigenic sequence. In an alternative embodiment, the invariant chain is indirectly linked to the antigenic sequence. Suitably the invariant chain is indirectly linked to the antigenic sequence by a peptide sequence. Suitably the peptide sequence comprises or more suitably consists of glycine and serine, more suitably the peptide sequence comprises or more suitably consists of the sequence GlySer. Alternatively, the peptide sequence comprises or consists of the ‘Ascl’ linker, which is a linker having the polypeptide sequence ArgArgAla, encoded by polynucleotide sequence AGGCGCGCC. Alternatively, the peptide sequence comprises or more suitably consists of the ‘res’ linker, which is a linker having the polypeptide sequence SerAspArgTyrLeuAsnArgArgAla (SEQ ID NO: 117), encoded by polynucleotide sequence AGCGATCGCTATTTAAATAGGCGCGCC (SEQ ID NO: 118). Alternatively, the peptide sequence comprises or more suitably consists of the human influenza hemagglutinin (HA) tag (polypeptide SEQ ID NO: 119, polynucleotide SEQ ID NO: 120).

A functional fragment of invariant chain is a portion of a full length invariant chain sequence which, when co-expressed with and operatively linked to antigen, enhances the immunogenic properties of the antigen beyond that which would have bene achieved without co-expression of the fragment of invariant chain. The enhancement in immunogenic properties may be increases in CD4+ and/or CD8+ and/or antibody responses. All ‘functional fragments of invariant chain’ referred to herein are functional in this respect.

Suitable functional fragments of invariant chain include those recited in WO2018037045. Suitably a functional fragment of invariant chain is a fragment of at least 10, more suitably 20, more suitably 30, more suitably 40, more suitably 50, more suitably 80, more suitably 150 amino acids of invariant chain which substantially maintains the properties described above. Alternatively, or in addition, a functional fragment of invariant chain is a polypeptide sequence sharing suitably at least 50% identity, more suitably 70% identity, more suitably 90% identity, more suitably 95% identity with a full length invariant chain sequence or a fragment of invariant chain sequence and substantially maintains the properties described above.

Suitably the functional fragment of invariant chain comprises or consists of a portion of residues 17-97 of SEQ ID NO: 1, wherein the portion comprises at least 5 contiguous residues from residues 77-92 of SEQ ID NO: 1 (human invariant chain p35 isoform), or the corresponding sequence from the invariant chain protein derived from another human invariant chain isoform or the invariant chain protein derived from an organism other than a human, such as those recited in SEQ ID NOs: 5, 9, 11, 13 and 15-52.

More suitably the functional fragment of invariant chain comprises or consists of residues 67-76, 68-77, 69-78, 70-79, 71-80, 72-81, 73-82, 74-83, 75-84, 76-85, 77-86, 78-87, 79-88, 80-89, 81-90, 82-91 or 83-92 of SEQ ID NO: 1; 67-81, 68-82, 69-83, 70-84, 71-85, 72-86, 73-87, 74-88, 75-89, 76-90, 77-91 or 78-92 of SEQ ID NO: 1; or 67-86, 68-87, 69-88, 70-89, 71-90, 72-91 or 73-92 of SEQ ID NO: 1.

Suitably the fragment of invariant chain refers to a truncated version of an invariant chain derived from an animal, such as a vertebrate, such as a fish, bird or mammal. Suitable truncated versions of invariant chain are provided in SEQ ID NOs: 4 and 53-116. More suitably the fragment of invariant chain refers to a truncated version of an invariant chain derived from a mammal. More suitably the fragment of invariant chain refers to a truncated version of an invariant chain derived from a mammal selected from the list consisting of a chicken, cow, dog, mouse, rat, non-human primate or human. More suitably the fragment of invariant chain refers to a truncated version of an invariant chain derived from a human or mouse. More suitably the fragment of invariant chain refers to a truncated version of an invariant chain derived from a human.

Different invariant chain sequences from various species are provided in SEQ ID NOs: 1, 5, 9, 11, 13 and 15-52. These invariant chain sequences or fragments or variants thereof, are all suitable for use in the present invention. Suitably the fragment of invariant chain is derived from mouse p31 or p41 isoforms of invariant chain. A particularly suitably fragment of invariant chain derived from mouse invariant chain is provided in SEQ ID NO: 4 (mli(1-75)K63R).

Suitably the functional fragment of invariant chain comprises or consists of a portion of residues 1-80 of SEQ ID NO: 11, wherein the portion comprises at least 10 contiguous residues from residues 50-75 of SEQ ID NO: 1.

In particular, the portion above may comprise or more suitably consist of residues 53-75, 55-75, 56-75, 60-75, 62-75 or 68-75 of SEQ ID NO: 11. Alternatively, the portion above may comprise or more suitably consist of residues 50-73, 50-70 or 50-65 of SEQ ID NO: 11. More suitably, the portion above may comprise or more suitably consist of residues 55-75 or 60-75 of SEQ ID NO: 11.

All references above to invariant chain are also equally applicable to functional fragments of invariant chain. As used herein ‘an invariant chain sequence’ refers to either the full length sequence of invariant chain, or a functional fragment or variant of invariant chain.

In one embodiment of the invention, the antigen may be co-expressed (at the N-terminus or C-terminus of the antigen) with flagellin or a functional fragment thereof.

Flagellin represents a pathogen associated molecular pattern (PAMP) that can interact with the TLR5 receptor as well as with at least two cytosolic PRR receptors. Fusion of flagellin to an antigen is a way to potentially make the antigen more immunologically potent and therefore effective. Without wishing to be bound by theory, it is thought that flagellin works by binding Toll-like receptor 5 (TLR5) which is present on cells of the innate immune system. TLRs recognize certain ‘patterns’ that are conserved in flagellin. Binding of flagellin to the TLR5 receptor triggers a series of innate and adaptive immune responses that are necessary for orchestration of an effective immune response.

### Antigens

The methods of the present invention involve the administration of antigens, either presented in polypeptide form, or encoded by nucleic acids comprised within viral vectors and/or oncolytic viruses. Antigen is also referred to herein as ‘antigenic sequence’ and ‘polypeptide antigen’. Without wishing to be bound by theory, it is believed that such antigens are delivered to cancer cells by the oncolytic virus and that these antigens then ‘mark’ target cancer cells for destruction by the immune system. The immune system effectively recognises the antigen due to the prior or subsequent administration of the viral vector comprising a nucleic acid encoding the antigen, or due to the prior or subsequent administration of the protein antigen.

Suitably the antigen is exogenous with respect to the viral vector. Suitably the antigen is exogenous with respect to the oncolytic virus. Suitably the antigen is exogenous with respect to the viral vector and the oncolytic virus. By exogenous it is meant that the antigen is not encoded by the virus in nature. Alternatively, the antigen may be native to the viral vector and/or the antigen may be native to the oncolytic virus.

The antigen is suitably derived from a bacterium, a virus or a parasite.

The antigen comprised within the viral vector and the antigen comprised within the oncolytic virus are the same antigen. Herein, two antigens are considered to be the same antigen if they comprise at least one cross-reacting epitope in common. More suitably, they comprise antigens sharing at least 50% sequence identity, more suitably 70% sequence identity, more suitably 90% sequence identity, more suitably 99% sequence identity, more suitably identical sequences.

Suitably the antigen comprises a CD8+, CD4+ and/or antibody epitope. More suitably the antigen comprises a CD8+ epitope.

Suitably the antigen is a non-self antigen. Alternatively, the antigen is a self antigen, more suitably a neoantigen, more suitably a tumour-associated antigen (TAA). In one embodiment, the antigen is not a neoantigen or a tumour-associated antigen (TAA). In another embodiment, the antigen is not ovalbumin (OVA). In one embodiment, the antigen is not HPV E6 or E7.

Exemplary antigens include proteins or fragments thereof (or encoded proteins or fragments thereof in a viral vector or oncolytic virus) from a pathogenic organism, e.g., a bacterium or virus or other microorganism, as well as proteins or fragments thereof from a cell, e.g., a cancer cell. Antigens could also include proteins or fragments thereof which are not from a pathogenic organism, such as ovalbumin (‘OVA’, with an example sequence provided as SEQ ID NO: 8).

Suitably the antigen is derived from a virus. Suitably the antigen is derived from HPV, HBV, hepatitis C Virus (HCV), retroviruses such as human immunodeficiency virus (HIV-1 and HIV-2), herpes viruses such as Epstein Barr Virus (EBV) and varicella zoster, cytomegalovirus (CMV), HSV-1 and HSV-2 or influenza virus. Particularly useful antigens include HBV surface antigen or HBV core antigen; HPV E6 and/or E7, ppUL83 or pp89 of CMV; antigens of gp120, gp41 or p24 proteins of HIV-1; ICP27, ICP4, gD, gB, gC, gE, gI antigens of HSV-1 or HSV-2; F, N, M antigens of RSV; or influenza hemagglutinin or nucleoprotein. Other antigens associated with pathogens that can be utilized as described herein are antigens of various parasites, including malaria, e.g., malaria peptide based on repeats of NANP.

In one embodiment, the antigen is selected from hepatitis B virus (HBV) core protein, hepatitis B virus (HBV) surface protein and human papilloma virus (HPV), e.g., E1, E2, E7 or E6 proteins.

Alternatively, the antigen is a variant of any of the above proteins. A variant is a protein (or encoding polynucleotide) sharing suitably at least 10%, more suitably at least 30%, more suitably at least 60%, more suitably at least 90% identity with the full-length original protein (or encoding polynucleotide).

In alternative embodiments, the antigen is from a pathogen that is a bacterium, such as Bordetella pertussis; Ehrlichia chaffeensis; Staphylococcus aureus; Toxoplasma gondii; Legionella pneumophila; Brucella suis; Salmonella enterica; Mycobacterium avium; Mycobacterium tuberculosis; Listeria monocytogenes; Chlamydia trachomatis; Chlamydia pneumoniae; Rickettsia rickettsii; or, a fungus, such as, e.g., Paracoccidioides brasiliensis; or other pathogen, e.g., Plasmodium falciparum or Plasmodium vivax.

It is not necessary to include a full-length antigen; it suffices to include an immunogenic fragment that will be presented by MHC class I and/or II and/or that contain B cell epitope.

It may be advantageous to analyse the genotype of the mammal and/or a tumour tissue sample from the mammal to select the appropriate choice of antigen before administration of the compositions.

### Administration

Administration according to the methods of the invention may be carried out in various ways.

The oncolytic virus or the viral vector may be administered at any therapeutically effective dosage amount. Therapeutically effective dosages may be about, 10³, about 10⁴, about 10⁵, about 10⁶, about 10⁷, about 10⁸, about 10⁹, about 10¹⁰, about 10¹¹, about 10¹² or about 10¹³ plaque forming units (pfu). Therapeutically effective dosages may also be about at least 10³, such as at least about 10⁴, such as at least about 10⁵, such as at least about 10⁶, such as at least about 10⁷, such as at least about 10⁸, such as at least about 10⁹, such as at least about 10¹⁰, such as at least about 10¹¹, such as at least about 10¹² or such as at least about 10¹³ plaque forming units (pfu).

Alternative therapeutically effective dosages may be at least about 10² viral particles (vp), such as at least about 10³ vp, such as at least about 10⁴ vp, such as at least about 10⁵ vp, such as at least about 10⁶ vp, such as at least about 10⁷ vp, such as at least about 10⁸ vp, such as at least about 10⁹ vp, such as at least about 10¹⁰ vp, such as at least about 10¹¹ vp, such as at least about 10¹² vp or such as at least about 10¹³ vp.

The antigen may be administered at any therapeutically effective dosage amount. Therapeutically effective dosages may be at least about 0.1 μg, such as at least about 0.5 μg, such as at least about 1 μg, such as at least about 2 μg, such as at least about 5 μg, such as at least about 10 μg, such as at least about 20 μg, such as at least about 50 μg, such as at least about 100 μg.

In the context of the first composition, a therapeutically effective dosage amount is a dose of antigen or viral vector comprising a nucleic acid encoding said antigen, which results in an immune response against the antigen. In the context of the second composition, a therapeutically effective dosage amount is a dose of oncolytic virus which results in the infection of at least one cancer cell by the oncolytic virus, more suitably a substantial proportion of the cancer cells in the mammal.

The compositions may be administered by a variety of modes of administration, including systemic, topical or localized administration.

In one embodiment, the compositions are each administered via mucosal administration, intravenous administration, intramuscular administration, intraperitoneal administration, subcutaneous administration, oral administration, rectal administration, intravaginal administration, intranasal administration, transmucosal administration or transdermal administration. Each composition may be administered via a different route.

The term “systemic administration” refers to administration of a composition in a manner that results in the introduction of the composition into the mammal's circulatory system or otherwise permits its spread throughout the body. “Regional” administration refers to administration into a specific, and limited, anatomical space, such as intraperitoneal, intrathecal, subdural, or to a specific organ. “Local administration” refers to administration of a composition into a limited, or circumscribed, anatomic space, such as intratumoral injection into a tumour mass, or peritumoral injection, subcutaneous injections, intradermal, intramuscular, or intravaginal injections. The compositions may be administered via any of these routes. The compositions may be administered at the site of tumour removal.

Suitably the compositions are administered by local administration, for example to mucosal tissue. Those of skill in the art will understand that local administration may also result in entry of a composition into the circulatory system i.e., rendering it systemic to one degree or another. Particular routes of administration include oral, intranasal, intrapulmonary, rectal or vaginal. Most suitably the compositions, and in particular the second composition, are administered into a tumour or topically at the site of tumour excision. In a preferred embodiment, the first composition is administered by a systemic administration and the second composition is administered by a local administration. In a more preferred embodiment, the first composition is administered by intramuscular injection and the second composition is administered by intratumoral or peritumoral injection. Suitably, when the cancer or tumour is localised at several sites, for example because of metastases, the second composition is administered by intratumoral or peritumoral injection to a single tumour site.

In one embodiment, administration of the second composition is carried out by direct injection into target tissue, which may be a tumour. The amount of virus administered is typically in the range of from 10⁴ to 10¹³ plaque forming units (pfu), preferably from 10⁵ to 10¹² pfu, more preferably about 10⁶ to 10¹² pfu. Typically, up to 500 μl, typically from 1 to 200 μl suitably from 1 to 10 μl of a pharmaceutical composition of the virus and a pharmaceutically acceptable suitable carrier or diluent would be used for injection. However, for some oncolytic therapy applications larger volumes up to, but not limited to 10 ml may also be used, depending on the tumour and the inoculation site.

The routes of administration and dosages described are intended only as a guide since a skilled practitioner will be able to determine readily the optimum route of administration and dosage. The dosage may be determined according to various parameters, especially according to the location of the tumour, the size of the tumour, the age, weight and condition of the patient to be treated and the route of administration. The dosage and dosage frequency may first be optimized pre-clinically by studying the properties of the virus in tissue culture and in a suitable animal model.

Suitably, the oncolytic virus is administered by direct injection into the tumour. The virus may also be administered systemically or by injection into a blood vessel supplying the tumour. The virus may also be administered as an intravesical treatment; such as might be used for treatment of cancers of the bladder. The optimum route of administration will depend on the location and size of the tumour.

Depending on the route of administration, the composition may be coated in a material to protect the compound from the action of enzymes, acids and other natural conditions which may inactivate the compound. Thus, it may be necessary to coat the composition with, or co-administer the composition with, a material to prevent its inactivation. For example, enzyme inhibitors of nucleases or proteases (e.g., pancreatic trypsin inhibitor, diisopropylfluorophosphate and trasylol) or in an appropriate carrier such as liposomes (including water-in-oil-in-water emulsions as well as conventional liposomes.

The compositions used in the methods of the invention can be administered in combination with other types of cancer treatment strategies (e.g., radiation therapy, chemotherapy, hormonal therapy, immunotherapy and anti-tumour agents). Examples of anti-tumour agents include, but are not limited to, cisplatin, ifosfamide, paclitaxel, taxanes, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, and taxo.

The compositions may be administered simultaneously (such as by co-administration, either as separate compositions or in co-formulation) or sequentially, suitably along with one or more further components such as therapeutically useful compounds or molecules such as antigenic proteins optionally simultaneously administered with adjuvant. Examples of co-administration include homo-lateral co-administration and contra-lateral co-administration. For example, the compositions can be administered (e.g. via an administration route selected from intramuscular, transdermal, intradermal, sub-cutaneous) to the same side or extremity (“co-lateral” administration) or to opposite sides or extremities (“contra-lateral” administration). Suitably the first composition is administered first, followed by the second composition. Alternatively, the second composition may be administered first, followed by the first composition. The first and/or second composition may each be administered multiple times. A series of administrations of the first and/or second composition may be performed such as administration of the first composition, followed by the second, followed by repeating the first. The first composition may be administered multiple times before the second composition is administered.

“Simultaneous” administration suitably refers to administration as substantially the same time. In one embodiment, both compositions are administered at the same time, however, one composition could be administered within a few minutes (for example, at the same medical appointment or doctor's visit), within a few hours. Such administration is also referred to as co-administration. Suitably if the compositions are administered simultaneously, they are co-formulated into one composition. Each composition may alternatively be formulated separately in which case, they may be administered co-locally at or near the same site.

The compositions may be administered as part of a series of administrations. Suitably the first composition is administered as part of a series of administrations of compositions, wherein the compositions other than the first composition administered in the series are homologous with respect to the first composition. Alternatively, the first composition is administered as part of a series of administrations of compositions, wherein one or more compositions other than the first composition administered in the series is a heterologous composition with respect to the first composition.

Suitably the heterologous composition comprises a different viral vector to the viral vector in the first composition. Suitably the heterologous composition comprises a different encoded antigen to that of the viral vector in the first composition. Suitably the heterologous composition comprises a different antigen to that of the first composition. Suitably the heterologous composition comprises an antigen when the first composition comprises a viral vector comprising a nucleic acid encoding the antigen. Suitably the heterologous composition comprises a viral vector comprising a nucleic acid encoding the antigen and the first composition comprises the antigen.

Suitably the second composition is administered as part of a series of administrations of compositions, wherein the compositions other than the second composition administered in the series are homologous with respect to the second composition. Suitably the second composition is administered as part of a series of administrations of compositions, wherein one or more compositions other than the second composition administered in the series is a heterologous composition with respect to the second composition.

Suitably the heterologous composition comprises a different oncolytic virus to the oncolytic virus in the second composition. Suitably the heterologous composition comprises a different encoded antigen to that of the oncolytic virus in the second composition.

In particular, the first composition in the form of a viral vector comprising a nucleic acid encoding the antigen may be administered, followed by one or more administrations of the first composition comprising the protein antigen, or vice-versa. This is preceded by, or followed by, the administration of the second composition.

In a particular embodiment the first composition is administered, followed by after approximately two weeks a further administration of the first composition, wherein the further administration is concomitant with administration of the second composition. Further administrations of the second composition may follow.

A prime-boost regimen may be used for administration of the first composition. Prime-boost refers to two separate immune responses: (i) an initial priming of the immune system followed by (ii) a secondary or boosting of the immune system many weeks or months after the primary immune response has been established. The first composition may be administered as a prime and then administered again as a boost, wherein when administered as a boost the first composition is heterologous to the first composition administered as the prime. The prime may be multiple administrations of the composition (such as two, three or four administrations) and the boost may be multiple administrations of the composition (such as two, three or four administrations). Preferably, a boosting composition is administered about 2 to about 27 weeks after administering the priming composition to the mammal.

Suitably the first composition is administered as a prime vaccination. Suitably the first composition is administered as a boost vaccination. Suitably the prime and/or the boost is administered multiple times.

If one or more priming and/or boosting steps are used, this step may include a single dose that is administered hourly, daily, weekly or monthly, or yearly. As an example, mammals may receive one or two doses containing between about 10 μg to about 50 μg of each composition. The amount or site of delivery is desirably selected based upon the identity and condition of the mammal. By the term “subject” is meant any animal, suitably a mammal, and preferably a human.

Suitably, administration of the first and/or second composition elicits a CD8+ T-cell response, a CD4+ T-cell response, and/or a B-cell response. More suitably administration of the first and/or second composition elicits a CD8+ T-cell response. More suitably still, administration of the first and/or second composition elicits a tumour infiltrating lymphocyte (TILs) response, in particular CD8+ TILs.

Suitably, administration of the first and/or second composition leads to differential expression of genes within the tumour microenvironment that are indicative of the presence of certain immune cell types and their respective functions (NK cell functions, T-cell functions, macrophage functions, etc.) and/or are involved in immunological and inflammation pathways (interferon, cytokines & receptors, etc.).

Suitably, administration of the first and/or second composition leads to differential expression of genes within the tumour microenvironment involved in one or more gene sets that characterise a particular pathway of anti-cancer immunity.

Gene set expression and enrichment scores can be assessed by NanoString technology as described in Example 1, or in the white paper from NanoString Technologies, Inc., Seattle, Wash. 98109 “Multiplexed Cancer Pathway Analysis” (MK1191, April 2019, Lucas Dennis, Patrick Danaher, Rich Boykin, Christina Bailey and Joseph Beechem). See also https://www.nanostring.com/scientifiic-content/technology-overview/ncounter-technology. Gene sets defined in the human nCounter® PANCancer Immune Profiling Panel are Adhesion, Antigen Processing, B-Cell Functions, Cell Cycle, Cell Functions, Chemokines, Complement, Cytokines, Cytotoxicity, Interleukins, Leukocyte Functions, Macrophage Functions, Microglial Functions, NK Cell Functions, Pathogen Defense, Regulation, Senescence, T-Cell Functions, TLR, TNF Superfamily and Transporter Function. The genes which are part of each of these gene sets are listed below:
Adhesion: ALCAM, CEACAM1, CEACAM6, CEACAM8, EPCAM, ICAM1, ICAM2, ICAM3, ICAM4, ITGA1, ITGA2, ITGA2B, ITGA4, ITGA5, ITGA6, ITGAE, ITGAL, ITGAM, ITGAX, ITGB1, ITGB2, ITGB3, ITGB4, MCAM, VCAM1.
Antigen Processing: CD1E, CD8A, HLA-A, HLA-B, HLA-C, HLA-DMA, HLA-DMB, HLA-DOB, HLA-DPA1, HLA-DPB1, HLA-DQA1, HLA-DQB1, HLA-DRA, HLA-DRB3, HLA-DRB4, MR1, PSMB7, PSMB9, TAP1, TAP2, TAPBP, THBS1.
B-Cell Functions: ADA, BLK, CD19, CD1D, CD27, CD274, CD38, CD3E, CD5, CD70, CD79B, CD80, CD86, CR2, CTLA4, CXCR5, FAS, IL11, IRF4, MS4A1, PTPRC, RAG1, SOCS1, TNFRSF14, TNFSF18.
Cell Cycle: ABL1, ATM, BAX, BCL2, BID, BIRC5, CASP3, CCND3, CDKN1A, CXCR4, NUP107, THBS1, TNFSF10.
Cell Functions: ADORA2A, AKT3, ANP32B, BATF, BTLA, CD1A, CD1B, CD1D, CD209, CD274, CD3E, CD6, CD68, CD70, CHIT1, CSF3, CXCR4, DOCK9, EPCAM, EWSR1, F13A1, FEZ1, FUT5, GATA3, GTF3C1, GZMA, GZMB, GZMH, GZMK, GZMM, HAVCR2, HSD11B1, ICOS, ICOSLG, IFNGR1, IL17RA, IL21R, IL3RA, KIT, KLRF1, LAIR2, LAMP3, LIF, LTK, MAF, MME, MPPED1, MSR1, NCR1, NEFL, NRP1, OSM, PDGFC, PLA2G6, PMCH, PSEN1, PTGDR2, REPS1, RORC, RPS6, RRAD, SMAD2, SMPD3, SOCS1, SYT17, TARP, TNFRSF17, TNFSF18, TPSAB1, USP9Y, ZNF205.
Chemokines: A2M, C1QBP, CCL1, CCL11, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL7, CCL8, CCR1, CCR3, CCR4, CCR7, CCRL2, CEACAM8, CKLF, CMKLR1, CSF2RB, CX3CL1, CX3CR1, CXCL1, CXCL10, CXCL11, CXCL12, CXCL13, CXCL14, CXCL16, CXCL2, CXCL3, CXCL5, CXCL6, CXCL9, CXCR1, CXCR2, CXCR3, CXCR4, CXCR5, CXCR6, IFI16, IFI27, IFI35, IFIT1, IFIT2, IFNAR2, IFNL2, IL11RA, IL13RA2, IL15RA, IL17B, IL17RB, IL19, IL1B, IL22RA1, IL22RA2, IL2RG, IL32, IL6ST, IL8, ILF3, IRF1, IRF2, IRF8, KLRB1, LTA, LTBR, MS4A2, PPBP, PSMB8, STAT1, STAT2, STAT3, STAT4, STAT5B, STATE, TNFRSF13B, TNFRSF1A, TNFRSF1B, TNFSF12, TNFSF15, TNFSF4, XCL2, XCR1.
Complement: C1QA, C1QB, C1R, C1S, C2, C4B, C4BPA, C5, C6, C7, CBA, C8B, C8G, C9, CCL25
Cytokines: CCL3L1, CCL5, CCR1, CCR2, CCR4, CCR5, CD70, CSF2, CSF3R, CXCL10, EBI3, FLT3LG, FOXP3, HLA-DOB, IDO1, IFNG, IFNL1, IL10RA, IL11, IL12A, IL12B, IL12RB2, IL13, IL13RA1, IL17A, IL1A, IL1B, IL1R2, IL1RN, IL2, IL21, IL22, IL23R, IL24, IL26, IL2RB, IL4R, IL5, IL5RA, IL6R, IL7R, IL8, IL9, JAK1, JAK2, JAK3, LTB, NOD2, OAS3, PTGS2, SPP1, TNFSF10, TNFSF14, TNFSF8, TYK2, VEGFA.
Cytotoxicity: GNLY, GZMA, GZMB, GZMH, GZMK, GZMM, HLA-A, HLA-B, HLA-C, PRF1.
Interleukins: IFNA1, IFNA17, IFNA2, IFNA7, IFNA8, IFNG, IFNL1, IL10, IL11, IL12A, IL12B, IL13, IL15, IL16, IL17A, IL17B, IL17F, IL18, IL19, IL1A, IL1B, IL1RN, IL21, IL23A, IL24, IL25, IL26, IL27, IL32, IL34, IL4, IL5, IL6, IL7, IL8, TGFB1, TGFB2, TNF.
Leukocyte Functions: CX3CL1, FUT7, HCK, IFNG, LCP1, SH2D1B, THBD, VEGFA.
Macrophage Functions: CD47, CD80, CD86, CSF2, DPP4, F2RL1, IFNG, LBP, LCP1, PRKCE, PSEN2, SBNO2, SLC11A1, SYK, TICAM1.
Microglial Functions: CX3CR1, TLR1, TLR3, TLR4, TLR7.
NK Cell Functions: CCR1, CD2, CD7, CXCL11, CXCR3, IFNG, IL12A, IL12B, IL12RB1, IL12RB2, IL18, IL18R1, IL18RAP, IRF1, ITGA1, KIR_Activating_Subgroup_1, KIR_Activating_Subgroup_2, KIR_Inhibiting_Subgroup_1, KIR_Inhibiting_Subgroup_2, KIR3DL1, KIR3DL2, KIR3DL3, KLRB1, KLRC1, KLRC2, KLRD1, KLRF1, KLRG1, KLRK1, LILRB1, NCR1.
Pathogen Defence: CCL22, CD8A, CTSG, CXCL10, GNLY, IFNAR1, IL1B, IL8, OAS3, PRF1, PRG2, TYK2.
Regulation: ABL1, AMBP, AMICA1, BAX, BCL6, BID, C3, C3AR1, CARD11, CASP3, CCL16, CCL19, CCL21, CCL23, CCL24, CCL3, CCL4, CCL8, CCR1, CCR4, CCR7, CD160, CD19, CD200, CD247, CD276, CD34, CD38, CD3D, CD3EAP, CD3G, CD40, CD40LG, CD47, CD5, CD7, CD80, CD81, CD86, CD8B, CD96, CDH1, CDKN1A, CMA1, COL3A1, CSF2, CTSG, CXCL1, CXCL10, CXCL2, CXCL3, CXCL6, CXCL9, CXCR1, CXCR2, CXCR4, DPP4, EGR2, ELANE, FAS, FCER1G, FCGR2B, FCGR3A, HLA-A, HLA-B, HLA-C, HLA-DMA, HLA-E, HLA-G, HMGB1, ICAM1, ICAM2, ICAM3, ICAM4, ICOSLG, IFITM1, IL12A, IL15, IL1B, IL2, IL2RA, IL3, IL4, IL5, IL8, INPP5D, IRF1, IRF2, IRF4, IRF8, ITGA4, ITGAL, ITGB1, ITGB2, JAK1, JAK2, JAK3, KIR_Activating_Subgroup_1, KIR_Activating_Subgroup_2, KIR_Inhibiting_Subgroup_1, KIR_Inhibiting_Subgroup_2, KIR3DL1, KIR3DL2, KIR3DL3, KLRC1, KLRD1, KLRG1, KLRK1, LAG3, LCK, LILRA1, LILRB1, LILRB2, LILRB3, LYN, MICA, MICB, NFATC1, NFATC2, NFATC3, NOTCH1, PDCD1, PLA2G1B, PVR, REL, RELB, RORA, RUNX1, RUNX3, SELE, SELL, SH2B2, SMAD3, SPINK5, SPN, STAT1, STAT2, STAT3, STAT4, STAT5B, STAT6, TAD, TBX21, TCF7, TGFB1, THBS1, TNFRSF13C, TNFRSF14, TNFSF10, TNFSF13, TNFSF13B, TNFSF14, TYK2, ULBP2, VCAM1.
Senescence: ABL1, CD44, CDKN1A, EGR1, ETS1, FN1, HRAS, IGF1R, IRF5, PLAU, PRKCD, SERPINB2.
T-Cell Functions: ADA, AICDA, CCR1, CCR4, CCR5, CD1C, CD1D, CD2, CD27, CD274, CD38, CD3E, CD3G, CD47, CD5, CD7, CD70, CD80, CD86, CD8A, CD8B, CTLA4, CXCL10, CXCL11, CXCL9, CXCR3, CXCR5, DPP4, EGR1, EOMES, F2RL1, FAS, FOXP3, IDO1, IFNG, IL11, IL12A, IL12B, IL12RB1, IL12RB2, IL13, IL13RA1, IL13RA2, IL18, IL18R1, IL18RAP, IL2, IL3, IL4, IL4R, IL5, IRF1, IRF4, ITGA1, LAG3, LCK, LCP1, LILRB1, MAF, PTPRC, RAG1, SOCS1, STAT4, STAT6, TBX21, TIGIT, TNFRSF14, TNFSF14, TNFSF18, TP53.
TLR: MYD88, TLR1, TLR10, TLR2, TLR3, TLR4, TLR5, TLR6, TLR7, TLR8, TLR9.
TNF Superfamily: CD70, FAS, LTB, TNF, TNFAIP3, TNFRSF10B, TNFRSF10C, TNFRSF11A, TNFRSF11B, TNFRSF12A, TNFRSF13B, TNFRSF13C, TNFRSF14, TNFRSF17, TNFRSF18, TNFRSF1A, TNFRSF1B, TNFRSF4, TNFRSF8, TNFRSF9, TNFSF10, TNFSF11, TNFSF12, TNFSF13, TNFSF13B, TNFSF14, TNFSF15, TNFSF18, TNFSF4, TNFSF8.
Transporter Functions: ANXA1, APOE, ATG10, ATG16L1, ATG7, CD163, CD36, CD44, CD47, CRP, CTSW, FAS, FCGR2A, FYN, ITGAM, LAMP1, MERTK, MFGE8, NT5E, PECAM1, SIGLEC1, TNFSF11.

Suitably, administration of the first and/or second composition leads to differential expression of genes within the tumour microenvironment involved in one or more, suitably in two, three, four, five or more human gene sets selected from Adhesion, Antigen Processing, B-Cell Functions, Cell Cycle, Cell Functions, Chemokines, Complement, Cytokines, Cytotoxicity, Interleukins, Leukocyte Functions, Macrophage Functions, Microglial Functions, NK Cell Functions, Pathogen Defence, Regulation, Senescence, T-Cell Functions, TLR, TNF Superfamily and Transporter Function as defined in the human nCounter® PANCancer Immune Profiling Panel. In a preferred embodiment, administration of the first and/or second composition leads to differential expression of genes within the tumour microenvironment involved in one, two, three, four or five human gene sets selected from Antigen processing, Chemokines, Cytokines, Interleukins and T-cell function as defined in the human nCounter® PANCancer Immune Profiling Pane.

Suitably, administration of the first and/or second composition leads to an enrichment score assessed by NanoString technology as described in example 1, of above 1.5, suitably above 2 for least one, preferably at least two, at least three, at least four or at least five gene sets selected from Adhesion, Antigen Processing, B-Cell Functions, Cell Cycle, Cell Functions, Chemokines, Complement, Cytokines, Cytotoxicity, Interleukins, Leukocyte Functions, Macrophage Functions, Microglial Functions, NK Cell Functions, Pathogen Defence, Regulation, Senescence, T-Cell Functions, TLR, TNF Superfamily and Transporter Function as defined in the human nCounter® PANCancer Immune Profiling Panel. In a preferred embodiment, administration of the first and/or second composition leads to an enrichment score assessed by NanoString technology, of above 1.5, suitably above 2 for one, two, three, four or five human gene sets selected from Antigen processing, Chemokines, Cytokines, Interleukins and T-cell function as defined in the human nCounter® PANCancer Immune Profiling Panel.

### Formulation

The method of the invention involves the administration of a first composition and a second composition. Preferably these compositions are suitable for use as a vaccine (i.e. suitable for mammalian, specifically human, administration).

Compositions of the present invention can be formulated in any conventional manner using one or more physiologically acceptable excipients and/or diluents.

A composition comprising a pharmaceutically acceptable excipient is a pharmaceutical composition. As used herein “pharmaceutically acceptable excipient” includes any and all dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

Pharmaceutically acceptable diluents include saline and aqueous buffer solutions. Pharmaceutical compositions suitable for injection include sterile aqueous solutions (where water soluble) or dispersions and sterile powders for the extemporaneous preparation of sterile injectable solutions or dispersion. Isotonic agents, for example, sugars, polyalcohols such as mannitol, sorbitol, sodium chloride may be included in the pharmaceutical composition. In all cases, the compositions should suitably be sterile. It should be stable under the conditions of manufacture and storage and must include preservatives that prevent contamination with microorganisms such as bacteria and fungi. Dispersions can also be prepared in glycerol, liquid polyethylene glycols, and mixtures thereof and in oils. Under ordinary conditions of storage and use, these preparations may contain a preservative to prevent the growth of microorganisms.

The diluent can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms in the pharmaceutical composition can be achieved by various antibacterial and antifungal agents, for example, parabens, chlorobutanol, phenol, ascorbic acid, thimerosal, and the like.

Compositions may be formulated in dosage unit form for ease of administration and uniformity of dosage. Dosage unit form refers to physically discrete units suited as unitary dosages for a mammalian subject; each unit contains a predetermined quantity of active material (e.g., the viral vector and the oncolytic virus) calculated to produce the desired therapeutic effect, in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active compound for the treatment of, and sensitivity of, individual subjects.

For lung administration, aerosolized solutions are suitable. In a sprayable aerosol preparations, the active protein may be in combination with a solid or liquid inert carrier material. This may also be packaged in a squeeze bottle or in admixture with a pressurized volatile, normally gaseous propellant. The aerosol preparations can contain solvents, buffers, surfactants, and antioxidants in addition to the protein of the invention.

The delivery of virus to cancerous cells that are to be treated may be performed using naked virus or by encapsulation of the virus in a carrier, e.g, in nanoparticles, liposomes or other vesicles. The virus may be delivered in a targeted release form. For example, the virus may be encapsulated and released at the target site using various means, such as ultrasound, Specifically, the virus may be encapsulated in gas-filled microspheres (e.g. of 1-10 urn diameter) encapsulated by a biocompatible stabilized shell. The microspheres may then be destabilised at the site of the tumour using ultrasound as described in Greco et al 2010 Mol Ther 18(2):295-306.

Alternatively, the virus may be delivered via mesenchymal stem cells. The use of mesenchymal stem cells for such a purpose is described in Mader et al 2009 Clin Cancer Res 15(23):7246-7255 and Castleton et al 2014 Blood 123:1327-1335.

Administration of the compositions is preferably in a “therapeutically effective amount”, this being sufficient to show benefit to the individual. The actual amount administered, and rate and time-course of administration, will depend on the nature and severity of the tumour being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners.

An effective amount of composition may be between about 1 nanogram and about 1 gram per kilogram of body weight of the recipient, between about 0.1 μg/kg and about 10 mg/kg, between about 1 μg/kg and about 1 mg/kg. Dosage forms suitable for internal administration may contain (for the latter dose range) from about 0.1 μg to 100 μg of active ingredient per unit. The active ingredient may vary from 0.5 to 95% by weight based on the total weight of the composition.

For systemic administration, injection is preferred, including intratumoral, intramuscular, intravenous, intraperitoneal, and subcutaneous. For the purposes of injection, the pharmaceutical compositions of the present invention can be formulated in liquid solution, preferably in physiologically compatible buffers, such as Hank's solution or Ringer's solution. In addition, the pharmaceutical compositions may be formulated in solid form and redissolved or suspended immediately prior to use. Lyophilized forms of the composition are also suitable.

The compositions can be formulated for parenteral administration by injection, e.g. by bolus injection or continuous infusion. Formulations for injection can be presented in a unit dosage form, e.g. in ampoules or in multi-dose containers, with an optionally added preservative. The compositions can further be formulated as suspensions, solutions or emulsions in oily or aqueous vehicles, and may contain other agents including suspending, stabilizing and/or dispersing agents.

Systemic administration can also be by transmucosal or transdermal means. For transmucosal or transdermal administration, penetrants appropriate to the barrier to be permeated may be used in the formulation. Such penetrants are generally known in the art, and include, for example, for transmucosal administration, bile salts, and fusidic acid derivatives. In addition, detergents may be used to facilitate permeation. Transmucosal administration can occur using nasal sprays or suppositories. Suitably the composition is in the form of a topical composition For topical administration, the oncolytic virus or the viral vector can be formulated into liquids such as ointments, salves, gels, or creams as generally known in the art. A wash solution can also be used locally.

### Cancer

The methods of the invention may be for the treatment of cancer, such as any solid tumour, suitably in a mammal, most suitably in a human. The first and/or second composition for use of the invention are suitably for use in the treatment of cancer.

As used herein, the term “cancer” includes, but is not limited to, neoplasms such as solid tumours and blood borne tumours. The term cancer includes diseases of the skin, tissues, organs, bone, cartilage, blood and vessels. A term used to describe cancer that is far along in its growth, also referred to as “late stage cancer” or “advanced stage cancer,” is cancer that is metastatic, e.g., cancer that has spread from its primary origin to another part of the body.

The viruses of the invention may be used in a mammal, suitably a human, in need of treatment (also referred to as a ‘patient’ or ‘subject’). A patient in need of treatment is an individual suffering from (or suitably at risk of suffering from) cancer, more suitably an individual having a solid tumour or believed to be at risk of having a tumour. The aim of therapeutic treatment is to improve the condition of a patient. Typically, therapeutic treatment using a method of the invention alleviates one or more symptoms of the cancer, such as reduction in its size (or mass), or substantial elimination of a tumour.

In one embodiment, the method of the invention treats a patient suffering from cancer or having a tumour in need of treatment.

Carrying out the method of the invention on an individual suffering from a tumour will typically kill the cells of the tumour thus decreasing the size of the tumour and/or preventing spread of malignant cells from the tumour while also recruiting antigen presenting cells (APCs) to the tumour site and inducing a protective anti-tumour immune response. Accordingly, in one embodiment, the methods of the present invention are suitable for the prevention of metastasis.

The methods of the present invention are suitable for the treatment of benign and malignant neoplasms (cancer).

Cancers that may treated according to the invention include, but are not limited to, cancer cells of the anus, bladder, blood, bone, bone marrow, brain, breast, colon, esophagus, gastrointestine, gum, head, kidney, liver, lung, nasopharynx, neck, oral cavity, oropharynx, ovary, penis, prostate, skin, stomach, testis, tongue, cervix, uterus, vagina or vulva.

The cancer may specifically be of the following histological type: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumour, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumour, malignant; thecoma, malignant; granulosa cell tumour, malignant; and roblastoma, malignant; Sertoli cell carcinoma; leydig cell tumour, malignant; lipid cell tumour, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumour, malignant; mullerian mixed tumour; nephroblastoma; hepatoblastoma; carcinosarcoma; mesenchymoma, malignant; brenner tumour, malignant; phyllodes tumour, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumour of bone; ewing's sarcoma; odontogenic tumour, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumour; meningioma, malignant; neurofibrosarcoma; neurilemmoma, malignant; granular cell tumour, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythro leukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In one embodiment the mammal has cancer, suitably a tumour, more suitably a solid tumour, for example a solid mucosal tumour.

The therapeutic levels of, or level of immune response against, the protein encoded by the selected antigen can be monitored to determine the need, if any, for boosters. Following an assessment of CD8+ T cell response, CD4+ T cell response and/or antibody titers, in the serum, optional booster administrations may be desired. Suitably the immune response elicited by the method of the invention is higher than that produced by the administration of the first or second compositions alone.

Treating cancer in a mammal includes eliminating the cancer, improving at least one symptom of the cancer or preventing or reducing the likelihood of the cancer to return. For example, treating a mammal having a tumour could be reducing the tumour mass e.g., by about 10%, 30%, 50%, 75%, 90% or more, eliminating the tumour, preventing or reducing the likelihood of the tumour to return, or partial or complete remission.

“Enhancing” an immune response includes inducing an immune response (i.e. an ab initio immune response in the absence of a previous immune response).

Recitation of ‘an’ does not imply the singular and includes the possibility of more than one of the stated entities.

### Clauses

Clauses further defining the invention are as follows:

1. A method of treating cancer in a mammal, said method comprising the steps of:
(i) administering to the mammal a first composition comprising a nucleic acid encoding an antigen and
(ii) administering to the mammal a second composition comprising an oncolytic virus wherein the oncolytic virus comprises a nucleic acid encoding the antigen.

2. A method of treating cancer in a mammal, said method comprising the steps of:
(i) administering to the mammal a first composition comprising an antigen and
(ii) administering to the mammal a second composition comprising an oncolytic virus wherein the oncolytic virus comprises a nucleic acid encoding the antigen.

3. A method of inducing an immune response to an antigen in a mammal, said method comprising the steps of:
(i) administering to the mammal a first composition comprising a nucleic acid encoding the antigen and
(ii) administering to the mammal a second composition comprising an oncolytic virus wherein the oncolytic virus comprises a nucleic acid encoding the antigen.

4. A method of inducing an immune response to an antigen in a mammal, said method comprising the steps of:
(i) administering to the mammal a first composition comprising the antigen and
(ii) administering to the mammal a second composition comprising an oncolytic virus wherein the oncolytic virus comprises a nucleic acid encoding the antigen.

5. A method of inducing an immune response to a cancer cell in a mammal, said method comprising the steps of:
(i) administering to the mammal a first composition comprising a nucleic acid encoding an antigen and
(ii) administering to the mammal a second composition comprising an oncolytic virus wherein the oncolytic virus comprises a nucleic acid encoding the antigen.

6. A method of inducing an immune response to a cancer cell in a mammal, said method comprising the steps of:
(i) administering to the mammal a first composition comprising an antigen and
(ii) administering to the mammal a second composition comprising an oncolytic virus wherein the oncolytic virus comprises a nucleic acid encoding the antigen.

7. A first composition comprising a nucleic acid encoding an antigen, said first composition for use in a method of inducing an immune response to the antigen in a mammal with a second composition comprising an oncolytic virus wherein the oncolytic virus comprises a nucleic acid encoding the antigen.

8. A second composition comprising an oncolytic virus wherein the oncolytic virus comprises a nucleic acid encoding an antigen, said second composition for use in a method of inducing an immune response to the antigen in a mammal with a first composition comprising a nucleic acid encoding the antigen.

9. A first composition comprising a nucleic acid encoding an antigen and a second composition comprising an oncolytic virus wherein the oncolytic virus comprises a nucleic acid encoding the antigen, said first and second compositions for use in a method of inducing an immune response to the antigen in a mammal.

10. A first composition comprising an antigen, said first composition for use in a method of inducing an immune response to the antigen in a mammal with a second composition comprising an oncolytic virus wherein the oncolytic virus comprises a nucleic acid encoding the antigen.

11. A second composition comprising an oncolytic virus wherein the oncolytic virus comprises a nucleic acid encoding an antigen, said second composition for use in a method of inducing an immune response to the antigen in a mammal with a first composition comprising the antigen.

12. A first composition comprising an antigen and a second composition comprising an oncolytic virus wherein the oncolytic virus comprises a nucleic acid encoding the antigen, said first composition for use in a method of inducing an immune response to the antigen in a mammal.

13. A first composition comprising a nucleic acid encoding an antigen, said first composition for use in the treatment of cancer in a mammal with a second composition comprising an oncolytic virus wherein the oncolytic virus comprises a nucleic acid encoding the antigen.

14. A second composition comprising an oncolytic virus wherein the oncolytic virus comprises a nucleic acid encoding an antigen, said second composition for use in the treatment of cancer in a mammal with a first composition comprising a nucleic acid encoding the antigen.

15. A first composition comprising a nucleic acid encoding an antigen and a second composition comprising an oncolytic virus wherein the oncolytic virus comprises a nucleic acid encoding the antigen, said first and second compositions for use in the treatment of cancer in a mammal.

16. A first composition comprising an antigen, said first composition for use in the treatment of cancer in a mammal with a second composition comprising an oncolytic virus wherein the oncolytic virus comprises a nucleic acid encoding the antigen.

17. A second composition comprising an oncolytic virus wherein the oncolytic virus comprises a nucleic acid encoding an antigen, said second composition for use in the treatment of cancer in a mammal with a first composition comprising the antigen.

18. A first composition comprising an antigen and a second composition comprising an oncolytic virus wherein the oncolytic virus comprises a nucleic acid encoding the antigen, said first and second compositions for use in the treatment of cancer in a mammal.

19. A first composition comprising a nucleic acid encoding an antigen, said first composition for use in a method of inducing an immune response to a cancer cell in a mammal with a second composition comprising an oncolytic virus wherein the oncolytic virus comprises a nucleic acid encoding the antigen.

20. A second composition comprising an oncolytic virus wherein the oncolytic virus comprises a nucleic acid encoding an antigen, said second composition for use in a method of inducing an immune response to a cancer cell in a mammal with a first composition comprising a nucleic acid encoding the antigen.

21. A first composition comprising a nucleic acid encoding an antigen and a second composition comprising an oncolytic virus wherein the oncolytic virus comprises a nucleic acid encoding the antigen, said first and second compositions for use in a method of inducing an immune response to a cancer cell in a mammal.

22. A first composition comprising an antigen, said first composition for use in a method of inducing an immune response to a cancer cell in a mammal with a second composition comprising an oncolytic virus wherein the oncolytic virus comprises a nucleic acid encoding the antigen.

23. A second composition comprising an oncolytic virus wherein the oncolytic virus comprises a nucleic acid encoding an antigen, said second composition for use in a method of inducing an immune response to a cancer cell in a mammal with a first composition comprising the antigen.

24. A first composition comprising an antigen and a second composition comprising an oncolytic virus wherein the oncolytic virus comprises a nucleic acid encoding the antigen, said first composition for use in a method of inducing an immune response to a cancer cell in a mammal.

25. Use of a first composition comprising a nucleic acid encoding an antigen, for the manufacture of a medicament for inducing an immune response to the antigen in a mammal with a second composition comprising an oncolytic virus wherein the oncolytic virus comprises a nucleic acid encoding the antigen.

26. Use of a second composition comprising an oncolytic virus wherein the oncolytic virus comprises a nucleic acid encoding an antigen, for the manufacture of a medicament for inducing an immune response to the antigen in a mammal with a first composition comprising a nucleic acid encoding the antigen.

27. Use of a first composition comprising a nucleic acid encoding an antigen and a second composition comprising an oncolytic virus wherein the oncolytic virus comprises a nucleic acid encoding the antigen, for the manufacture of a medicament for inducing an immune response to the antigen in a mammal.

28. Use of a first composition comprising an antigen, for the manufacture of a medicament for inducing an immune response to the antigen in a mammal with a second composition comprising an oncolytic virus wherein the oncolytic virus comprises a nucleic acid encoding the antigen.

29. Use of a second composition comprising an oncolytic virus wherein the oncolytic virus comprises a nucleic acid encoding an antigen, for the manufacture of a medicament for inducing an immune response to the antigen in a mammal with a first composition comprising an antigen.

30. Use of a first composition comprising an antigen and a second composition comprising an oncolytic virus wherein the oncolytic virus comprises a nucleic acid encoding the antigen, for the manufacture of a medicament for inducing an immune response to the antigen in a mammal.

31. Use of a first composition comprising a nucleic acid encoding an antigen, for the manufacture of a medicament for the treatment of cancer in a mammal with a second composition comprising an oncolytic virus wherein the oncolytic virus comprises a nucleic acid encoding the antigen.

32. Use of a second composition comprising an oncolytic virus wherein the oncolytic virus comprises a nucleic acid encoding an antigen, for the manufacture of a medicament for the treatment of cancer in a mammal with a first composition comprising a nucleic acid encoding the antigen.

33. Use of a first composition comprising a nucleic acid encoding an antigen and a second composition comprising an oncolytic virus wherein the oncolytic virus comprises a nucleic acid encoding the antigen, for the manufacture of a medicament for the treatment of cancer in a mammal.

34. Use of a first composition comprising an antigen, for the manufacture of a medicament for the treatment of cancer in a mammal with a second composition comprising an oncolytic virus wherein the oncolytic virus comprises a nucleic acid encoding the antigen.

35. Use of a second composition comprising an oncolytic virus wherein the oncolytic virus comprises a nucleic acid encoding an antigen, for the manufacture of a medicament for the treatment of cancer in a mammal with a first composition comprising the antigen.

36. Use of a first composition comprising an antigen and a second composition comprising an oncolytic virus wherein the oncolytic virus comprises a nucleic acid encoding the antigen, for the manufacture of a medicament for the treatment of cancer in a mammal.

37. Use of a first composition comprising a nucleic acid encoding an antigen, for the manufacture of a medicament for inducing an immune response to a cancer cell in a mammal with a second composition comprising an oncolytic virus wherein the oncolytic virus comprises a nucleic acid encoding the antigen.

38. Use of a second composition comprising an oncolytic virus wherein the oncolytic virus comprises a nucleic acid encoding an antigen, for the manufacture of a medicament for inducing an immune response to a cancer cell in a mammal with a first composition comprising a nucleic acid encoding the antigen.

39. Use of a first composition comprising a nucleic acid encoding an antigen and a second composition comprising an oncolytic virus wherein the oncolytic virus comprises a nucleic acid encoding the antigen, for the manufacture of a medicament for inducing an immune response to a cancer cell in a mammal.

40. Use of a first composition comprising an antigen, for the manufacture of a medicament for inducing an immune response to a cancer cell in a mammal with a second composition comprising an oncolytic virus wherein the oncolytic virus comprises a nucleic acid encoding the antigen.

41. Use of a second composition comprising an oncolytic virus wherein the oncolytic virus comprises a nucleic acid encoding an antigen, for the manufacture of a medicament for inducing an immune response to a cancer cell in a mammal with a first composition comprising the antigen.

42. Use of a first composition comprising an antigen and a second composition comprising an oncolytic virus wherein the oncolytic virus comprises a nucleic acid encoding the antigen, for the manufacture of a medicament for inducing an immune response to a cancer cell in a mammal.

43. The method according to any one of clauses 3 to 6 wherein the method is for the treatment of cancer.

44. The first composition for use according to any one of clauses 7 to 24 wherein the first composition is for use in the treatment of cancer.

45. The second composition for use according to any one of clauses 8 to 23 wherein the second composition is for use in the treatment of cancer.

46. The method, composition for use or use according to any one of clauses 1 to 45 wherein the first and second compositions are administered sequentially or simultaneously.

47. The method, composition for use or use according to clause 46 wherein the first and second compositions are administered simultaneously and separately.

48. The method, composition for use or use according to clause 46 wherein the first and second compositions are administered simultaneously and are co-formulated.

49. The method, composition for use or use according to clause 46 wherein the wherein the first and second compositions are administered sequentially and the first composition is administered first, followed by the second composition.

50. The method, composition for use or use according to clause 46 wherein the first and second compositions are administered sequentially and the second composition is administered first, followed by the first composition.

51. The method, composition for use or use according to any one of clauses 1 to 50 wherein the first composition is administered multiple times.

52. The method, composition for use or use according to any one of clauses 1 to 51 wherein the second composition is administered multiple times.

53. The method, composition for use or use according to any one of clauses 1 to 52 wherein the first composition is administered as part of a series of administrations of compositions, wherein the compositions other than the first composition administered in the series are homologous with respect to the first composition.

54. The method, composition for use or use according to any one of clauses 1 to 52 wherein the first composition is administered as part of a series of administrations of compositions, wherein one or more compositions other than the first composition administered in the series is a heterologous composition with respect to the first composition.

55. The method, composition for use or use according to clause 54 wherein the heterologous composition comprises a nucleic acid in a different form to that of the first composition.

56. The method, composition for use or use according to either clause 54 or 55 wherein the heterologous composition comprises a different encoded antigen to that of the nucleic acid in the first composition.

57. The method, composition for use or use according to either clause 54 or 55 wherein the heterologous composition comprises a different antigen to that of the first composition.

58. The method, composition for use or use according to any one of clauses 1 to 57 wherein the second composition is administered as part of a series of administrations of compositions, wherein the compositions other than the second composition administered in the series are homologous with respect to the second composition.

59. The method, composition for use or use according to any one of clauses 1 to 57 wherein the second composition is administered as part of a series of administrations of compositions, wherein one or more compositions other than the second composition administered in the series is a heterologous composition with respect to the second composition.

60. The method, composition for use or use according to clause 59 wherein the heterologous composition comprises a different oncolytic virus to the oncolytic virus in the second composition.

61. The method, composition for use or use according to either clause 59 or 60 wherein the heterologous composition comprises a different encoded antigen to that of the oncolytic virus in the second composition.

62. The method, composition for use or use according to any one of clauses 1 to 61 wherein the compositions are administered as part of a prime boost regimen.

63. The method, composition for use or use according to clause 62 wherein the first composition is administered as a prime vaccination.

64. The method, composition for use or use according to either clause 62 or 63 wherein the first composition is administered as a boost vaccination.

65. The method, composition for use or use according to any one of clauses 62 to 64 wherein the prime is administered multiple times.

66. The method, composition for use or use according to any one of clauses 62 to 65 wherein the boost is administered multiple times.

67. The method, composition for use or use according to any one of clauses 1 to 66 wherein the first composition is administered via mucosal administration, intravenous administration, intramuscular administration, intraperitoneal administration, subcutaneous administration, oral administration, rectal administration, intravaginal administration, intranasal administration, transmucosal administration or transdermal administration.

68. The method, composition for use or use according to clause 67 wherein the first composition is administered via mucosal administration.

69. The method, composition for use or use according to any one of clauses 1 to 68 wherein the second composition is administered via mucosal administration, intravenous administration, intramuscular administration, intraperitoneal administration, subcutaneous administration, oral administration, rectal administration, intravaginal administration, intranasal administration, transmucosal administration or transdermal administration.

70. The method, composition for use or use according to clause 69 wherein the second composition is administered via mucosal administration.

71. The method, composition for use or use according to any one of clauses 1 to 70 wherein the mammal is believed to be at risk of having a tumour.

72. The method, composition for use or use according to any one of clauses 1 to 70 wherein the cancer is a solid tumour.

73. The method, composition for use or use according to clause 72 wherein the solid tumour is metastatic.

74. The method, composition for use or use according to either clause 72 or 73, wherein the first composition is administered intratumorally.

75. The method, composition for use or use according to either clause 72 or 73, wherein the first composition is administered peritumorally.

76. The method, composition for use or use according to any one of clauses 72 to 75, wherein second composition is administered intratumorally.

77. The method, composition for use or use according to any one of clauses 72 to 75, wherein second composition is administered peritumorally.

78. The method, composition for use or use according to any one of clauses 1 to 71 wherein a tumour has been removed from the mammal.

79. The method, composition for use or use according to clause 78 wherein the first composition is administered at the site of tumour removal.

80. The method, composition for use or use according to either clause 78 or 79 wherein the second composition is administered at the site of tumour removal.

81. The method, composition for use or use according to either clause 79 or 80 wherein the first and second compositions are administered at the site of tumour removal.

82. The method, composition for use or use according to any one of clauses 1 to 81 wherein the antigen comprises a CD8+ T-cell epitope.

83. The method, composition for use or use according to any one of clauses 1 to 82 wherein the antigen comprises a CD4+ T-cell epitope.

84. The method, composition for use or use according to any one of clauses 1 to 83 wherein administration of the first composition elicits a CD8+ T-cell response.

85. The method, composition for use or use according to any one of clauses 1 to 84 wherein administration of the first composition elicits a CD4+ T-cell response.

86. The method, composition for use or use according to any one of clauses 1 to 85 wherein the nucleic acid in the first composition is a naked nucleic acid.

87. The method, composition for use or use according to any one of clauses 1 to 85 wherein the nucleic acid in the first composition is comprised within a vector.

88. The method, composition for use or use according to clause 87 wherein the vector is selected from a viral vector, a virus like particle (VLP), a self-amplifying RNA molecule or a bacterial vector.

89. The method, composition for use or use according to clause 88 wherein the vector is a viral vector.

90. The method, composition for use or use according to clause 89 wherein the antigen is exogenous with respect to the viral vector.

91. The method, composition for use or use according to clause 89 wherein the antigen is native to the viral vector.

92. The method, composition for use or use according to any one of clauses 1 to 91 wherein the antigen is exogenous with respect to the oncolytic virus.

93. The method, composition for use or use according to any one of clauses 1 to 91 wherein the antigen is native to the oncolytic virus.

94. The method, composition for use or use according to any one of clauses 1 to 93 wherein the antigen is derived from a bacterium, a virus or a parasite.

95. The method, composition for use or use according to any one of clauses 89 to 94 wherein the viral vector is non-replicating.

96. The method, composition for use or use according to any one of clauses 1 to 95 wherein the oncolytic virus is non-replicating.

97. The method, composition for use or use according to any one of clauses 89 to 94 and 96 wherein the viral vector is replication competent.

98. The method, composition for use or use according to any one of clauses 1 to 95 and 97 wherein the oncolytic virus is replication competent.

99. The method, composition for use or use according to any one of clauses 1 to 98 wherein the oncolytic virus is selected from adenovirus, adeno-associated virus, influenza virus, reovirus, vesicular stomatitis virus (VSV), Newcastle disease virus (NDV), vaccinia virus, poliovirus, measles virus, mumps virus, sindbis virus (SrN), paramyxovirus, poxvirus (such as vaccinia virus), picornavirus, herpesvirus and sendai virus (SV).

100. The method, composition for use or use according to any one of clauses 89 to 99 wherein the viral vector is selected from adenovirus, retrovirus, lentivirus, adeno-associated virus, herpesvirus, vaccinia virus (such as Modified Vaccinia Ankara (MVA)), foamy virus, cytomegalovirus, Semliki forest virus and poxvirus.

101. The method, composition for use or use according to any one of clauses 1 to 100 wherein the nucleic acid of the first composition comprises an invariant chain sequence.

102. The method, composition for use or use according to any one of clauses 1 to 101 wherein the nucleic acid of the second composition comprises an invariant chain sequence.

103. The method, composition for use or use according to either clause 101 or 102 wherein the invariant chain sequence is operatively linked to the antigen.

104. The method, composition for use or use according to any one of clauses 1 to 103 wherein the antigen is a non-self antigen.

105. The method, composition for use or use according to any one of clauses 1 to 103 wherein the antigen is a self antigen.

106. The method, composition for use or use according to clause 105 wherein the antigen is a neoantigen.

107. The method, composition for use or use according to any one of clauses 1 to 104 wherein the antigen is selected from a tumour-associated antigen (TAA), ovalbumin, HBV core, HPV E6 or HPV E7.

108. The method, composition for use or use according to any one of clauses 1 to 107 wherein the first composition comprises an adjuvant.

109. The method, composition for use or use according to any one of clauses 1 to 108 wherein the second composition comprises an adjuvant.

110. The method, composition for use or use according to either clause 108 or 109 wherein the adjuvant is selected from one or more of a saponin, a TLR4 agonist, a TLR7 agonist, a TLR8 agonist, a TLR9 agonist, 3D-MPL, GLA and CRX601.

111. The method, composition for use or use according to any one of clauses 108 to 110 wherein the adjuvant is in an emulsion formulation.

112. The method, composition for use or use according to any one of clauses 108 to 110 wherein the adjuvant is in a liposomal formulation.

113. The method, composition for use or use according to any one of clauses 108 to 110 wherein the adjuvant is in an ISCOM formulation.

114. The method, composition for use or use according to any one of clauses 1 to 113 wherein the first composition is in the form of a topical composition.

115. The method, composition for use or use according to clause 114 wherein the first composition is in the form of a liquid, such as a gel.

116. The method, composition for use or use according to any one of clauses 1 to 115 wherein the first composition is administered in targeted release form.

117. The method, composition for use or use according to clause 116 wherein the first composition is administered in encapsulated form and destabilised at the tumour site.

118. The method, composition for use or use according to any one of clauses 1 to 117 wherein the second composition is in the form of a topical composition.

119. The method, composition for use or use according to clause 118 wherein the second composition is in the form of a liquid, such as a gel.

120. The method, composition for use or use according to any one of clauses 1 to 119 wherein the second composition is administered in targeted release form.

121. The method, composition for use or use according to clause 120 wherein the second composition is administered in encapsulated form and destabilised at the tumour site.

122. The method, composition for use or use according to either clause 117 or 121 wherein the destabilisation is achieved by ultrasound.

123. The method, composition for use or use according to any one of clauses 1 to 122 wherein the viral vector is comprised within mesenchymal stem cells.

124. The method, composition for use or use according to any one of clauses 1 to 123 wherein the oncolytic virus is comprised within mesenchymal stem cells.

125. The method, composition for use or use according to any one of clauses 1 to 124 wherein the genotype of the mammal and/or a tumour tissue sample from the mammal are analysed to select the appropriate choice of antigen before administration of the compositions.

126. The method, composition for use or use according to any one of clauses 1 to 125 wherein the oncolytic virus comprises a nucleic acid encoding an immune system signalling molecule which is expressed only in tumour cells.

127. The method, composition for use or use according to clause 126 wherein the oncolytic virus comprises a nucleic acid encoding an immune system signalling molecule and targets tumour cells through the expression of a ligand which binds to a molecule preferentially expressed at the surface of tumour cells.

128. The method, composition for use or use according to any one of clauses 1 to 127, wherein

administration of the first and/or second composition leads to differential expression of genes within the tumour microenvironment that are indicative of the presence of certain immune cell types and their respective functions (such as NK cell functions, T-cell functions, macrophage functions, etc.) and/or are involved in immunological and inflammation pathways (such as interferon, cytokines & receptors, etc.).

129. The method, composition for use or use according to any one of clauses 1 to 128, wherein administration of the first and/or second composition leads to differential expression of genes within the tumour microenvironment involved in one or more gene sets that characterise a particular pathway of anti-cancer immunity.

130. The method, composition for use or use according to clause 129, wherein administration of the first and/or second composition leads to differential expression of genes within the tumour microenvironment involved in one or more, suitably in two, three, four, five or more human gene sets selected from Adhesion, Antigen Processing, B-Cell Functions, Cell Cycle, Cell Functions, Chemokines, Complement, Cytokines, Cytotoxicity, Interleukins, Leukocyte Functions, Macrophage Functions, Microglial Functions, NK Cell Functions, Pathogen Defence, Regulation, Senescence, T-Cell Functions, TLR, TNF Superfamily and Transporter Function as defined in the human nCounter® PANCancer Immune Profiling Panel.

131. The method, composition for use or use according to clause 130, wherein administration of the first and/or second composition leads to differential expression of genes within the tumour microenvironment involved in one, two, three, four or five human gene sets selected from Antigen processing, Chemokines, Cytokines, Interleukins and T-cell function as defined in the human nCounter® PANCancer Immune Profiling Pane.

132. The method, composition for use or use according to any one of clauses 1 to 131, wherein administration of the first and/or second composition leads to an enrichment score assessed by NanoString technology as described in example 1, of above 1.5, suitably above 2 for least one, preferably at least two, at least three, at least four or at least five gene sets selected from Adhesion, Antigen Processing, B-Cell Functions, Cell Cycle, Cell Functions, Chemokines, Complement, Cytokines, Cytotoxicity, Interleukins, Leukocyte Functions, Macrophage Functions, Microglial Functions, NK Cell Functions, Pathogen Defence, Regulation, Senescence, T-Cell Functions, TLR, TNF Superfamily and Transporter Function as defined in the human nCounter® PANCancer Immune Profiling Panel.

133. The method, composition for use or use according to clause 132, wherein administration of the first and/or second composition leads to an enrichment score assessed by NanoString technology, of above 1.5, suitably above 2 for one, two, three, four or five human gene sets selected from Antigen processing, Chemokines, Cytokines, Interleukins and T-cell function as defined in the human nCounter® PANCancer Immune Profiling Panel.

134. A kit comprising (i) a first composition comprising a nucleic acid encoding an antigen and (ii) a second composition comprising an oncolytic virus wherein the oncolytic virus comprises a nucleic acid encoding the antigen

135. A kit comprising (i) a first composition comprising an antigen and (ii) a second composition comprising an oncolytic virus wherein the oncolytic virus comprises a nucleic acid encoding the antigen.

### EXAMPLES

### Example 1

In this experiment, the goal was to assess the impact of combining a priming vaccination and intra-tumour MVA (Modified Vaccinia Ankara Virus) injection on tumour growth, immunogenicity and tumour immune infiltration by using a ChAd vector coding for the hepatitis B core (HBc) antigen together with administration of AS01-adjuvanted hepatitis B core & surface (HBc-HBs) protein antigens followed by two sequential intra-tumour injections of MVA coding for HBc and HBs antigens.

### Materials and Methods

### Investigational Products

The HBc antigen comprises a truncated HBV Core protein (SEQ ID NO: 121).
The HBs antigen is a commercially available antigen included in the formulation of HBs-containing vaccines Engerix-B, Twinrix/Ambirix, Infanrix hexa, Fendrix.
AS01 is an adjuvant formulation comprising QS21 and 3D-MPL in a liposomal formulation.
MVA-HBV is a replication-deficient recombinant MVA vector which contains a transgene encoding a truncated HBV Core protein antigen (HBc, SEQ ID NO: 122) and the full-length HBS Surface antigen (HBs, SEQ ID NO:123), separated by a self-cleaving 2A region.
ChAd155-hIi-HBV is a ChAd155 recombinant vector which contains a transgene encoding a truncated HBV Core protein antigen (HBc, SEQ ID NO: 124) fused N-terminally to a gene encoding a human MHC class II-associated invariant chain p35 isoform (hIi).

### Study Model

Animal model: 6-8 weeks old female BALB/c mice (Envigo NL) Tumour Cell Preparation: Cryo vials containing CT-26 tumour cells (Mus musculus (mouse) colon carcinoma fibroblasts) were thawed and cultured according to manufacturer's protocol. On the day of injection, cells were washed in serum free media, counted, and resuspended in cold serum free media at a concentration of 250,000 viable cells/100 μl. Cells were prepared for injections by withdrawing 100 μl cell suspension into a 1 ml syringe. The cell suspension and filled syringes were kept on ice.

Tumour Implantation: Animals were prepared for injection using standard approved anaesthesia, the mice were shaved prior to injection. One mouse at a time was immobilized and the site of injection disinfected with an alcohol swab. 100 μl of the cell suspension was subcutaneously injected into bilateral flank of the mouse. During implantation, a new syringe and needle was used for every mouse inoculated to minimize tumour ulceration. The cells were drawn up into a 1 mL syringe (no needle attached) to 150 μL with the 50 μL nearest to the plunger being air and 100 μL of cell suspension. Once the cells were drawn up, the needle was attached (without priming the needle). For implantation, the skin was lifted or tent using forceps to ensure a subcutaneous injection. For cell injection, the syringe/needle was twisted and then pulled out. Mice were ear tagged.

Tumour measurement: Animals were monitored for palpable tumours, or any changes in appearance or behaviour. Daily monitoring took place for mice showing any signs of morbidity or mortality. Once tumours were palpable, they were measured 3 times a week using electronic calipers. Tumour volume were calculated using the following equation: (longest diameter*shortest diameter²)/2. Once tumours were of appropriate size to begin the study, tumours and body weights were measured 3 times per week for the duration of the study. One individual was responsible for tumour measurements for the duration of the study. Termination of the study (tumour growth volume as endpoint) was defined as when individual tumour volume reached 3000 mm³ (any tumour side).

Randomization and dose selection: Since randomization was not possible due to vaccination occurring before tumour engraftment, 32 mice/group were initially engrafted with CT26 tumour cells to mitigate for variability of engraftment and tumour development. At the day of MVA dosing (Day 0), among the 32 animals/group, 16 mice were selected based on their right tumour size targeting a tumour volume within a predefined range of 80-120 mm3. The rationale of this selection was to decrease variability of initial tumour volume and ensure a certain degree of biological homogeneity, as it is known that large tumours might start to develop necrosis which might impact the overall effect of treatment.

Body weight: Body weight was measured 7 times a week following randomization and initiation of treatment. If body weight loss of >10% was observed, Dietgel could be given ad libitum. If body weight loss of >15% was observed, animal could be given a dosing holiday until weight loss was <10%. If body weight loss of >20% was observed, animal would be monitored daily for signs of recovery for up to 72 hours. If there were no signs of recovery, the animal would be sacrificed for humane reasons as per our IACUC protocol regulations.

Clinical Observations: Clinical observations were performed 3 times a week at the time of tumour and body weight measurements.

Tissue Collection: at day 9, 6 animals/group were sacrificed for collection of spleens (ex vivo stimulation) as well as for tumour collection (both sides). Tumours were cut into 2 halves where ½ was placed in RNA Later® and the other ½ was used for analysis of tumour infiltrating lymphocytes by FACS.

Euthanasia: Euthanasia was performed as follow: Isoflurane inhalation (2.5-4%), followed by cervical dislocation under anaesthesia once the mouse has been anesthetized as demonstrated by shallow breathing and no response to footpad pinch.

Moribundity: Any moribund animals were euthanized for humane reasons. Animals were terminated if tumour size measured greater than 3000 mm³. Animals were terminated if the animal had lost greater than 20% of its pre-treatment body weight and didn't recovered within 72 hours. Animals were terminated if there were any signs of distress due to tumour size, or ulcerations in the tumour or if the animal were unable to ambulate in order to obtain food and water. Signs of pain or distress are described as follows:
Changes in activity level (lethargy, recumbent position, delayed response to handling);
Changes in appearance (labored breathing, skin color, hunched posture, scruffy/ruffled fur);
Changes in vocalizations (chattering, whining, whimpering);
Changes in feeding behavior (weight loss, dehydration, emaciation).

Final Disposition: At termination of study, all living animals were euthanized and discarded.

Postmortem Study Evaluations: For every moribund animal a full necropsy was performed and documented at the time of sacrifice if possible.

### Immunological Read-Outs

### Cellular Immune Response—Intracellular Cytokine Staining (ICS)

The frequencies of vaccine-specific CD4+& CD8+ T-cells producing IL-2 and/or IFN-γ and/or TNF-α were evaluated in splenocytes collected at Day 9 after MVA injection for ex-vivo stimulation with HBs, HBc & HBc_F peptides pools. Peptide pools from B. pertussis were used as stimulation to evaluate non-specific response.

Isolation of splenocytes—Spleens are collected and placed in RPMI/additives (supplemented with Glutamine, Penicillin/streptomycin, Sodium Pyruvate, non-essential amino-acids and 2-mercaptoethanol). Cell suspensions are prepared from each spleen using a tissue grinder (Potter). Spleens are crushed in a potter with 4 ml cold complete medium. The splenic cell suspensions are filtered (Cell strainer 100 μm). The filter is rinsed with 40 ml cold RPMI/additives. After centrifugation (1300RPM, 10 min at RT), cells are resuspended in complete medium (RPMI supplemented with Glutamine, Penicillin/streptomycin, Sodium Pyruvate, non-essential amino-acids and 2-mercaptoethanol, and 5% Heat inactivated Fetal Calf Serum). Cells are counted (i.e, MACSQUANT).

In vitro stimulation of fresh splenocytes—Fresh splenocytes are plated in round bottom 96-well plates at approximately 1 million cells per well. Splenocytes are then stimulated for 6 hours (37° C., 5% CO2) with anti-CD28 (clone 37.51, BD REF 553294) and anti-CD49d (clone 9C10 (MFR4.B), BD REF 553313) at 1 μg/ml, with:
Condition 1: HBc peptide pool (stock @ 5 mg/ml/peptide) final working concentration for stimulation is 1 ug/ml (to be diluted 1:5000);
Condition 2: HBc F epitope peptide pool (stock @ 1 mg/ml/peptide) final working concentration for stimulation is 1 ug/ml (to be diluted 1:1000);
Condition 3: HBs peptide pool (stock @ 2 mg/ml/peptide) final working concentration for stimulation is 1 ug/ml (to be diluted 1:2000);
Condition 4: Un-relevant peptide (B-act or any other peptide pool available) final working concentration for stimulation is 1 ug/ml;
Condition 5: PMA/Ionomycine at respectively final concentrations of 0.25 ug/mL and 2.5 mg/ml;
Condition 6: Culture media (no stimulation)
   After a 2 hour-stimulation, Brefeldin A diluted 1/1000 in complete medium is added for 4 additional hours. Plates are then transferred at 4° C., overnight.

Intra-cellular staining (ICS)—Cells are stained and analyzed using a 5-colour ICS assay.

Cells are transferred to V-bottom 96-well plates, centrifuged at 189 g for 5 min at 4° C. and resuspended in 50 μl Flow Buffer (PBS 1×, 1% FCS) containing anti-CD16/32 (clone 2.4G2) diluted 1/50 for 10 min at 4° C. Then, 50 μl Flow Buffer containing anti-CD4-V450 (clone RM4-5) and anti-CD8-PerCp-Cy5.5 (clone 53-6.7) antibodies (diluted 1/100 and 1/50 respectively) and Live/dead-PO (1/500) is added for 30 min at 4° C. Cells are centrifuged (189 g for 5 min at 4° C.) and washed with 200 μl Flow Buffer.

Leukocytes are fixed and permeabilized by adding 200 μl of Cytofix/Cytoperm solution for 20 min at 4° C. Cells are centrifuged (189 g for 5 min at 4° C.) and washed with 200 μl Perm/Wash buffer. After an additional centrifugation step, cells are stained in 50 μl Perm/Wash buffer with anti-IL2-FITC (clone JES6-5H4, diluted 1/400), anti-IFN□-APC (clone XMG1.2, diluted 1/200) and anti-TNF□-PE (clone MP6-XT22, diluted 1/700) antibodies, for 2 hours at 4° C. Cells are washed twice with the Perm/Wash buffer suspended in 220 μl PBS. Stained cells are analyzed by flow cytometry using a LSRII and the FlowJo software. Percentage of CD4+ and CD8+ T cells secreting IFN-g and/or IL-2 and/or TNFα is calculated.

### Tumour Preparation and Flow Cytometry Analysis of Infiltrating Lymphocytes

Excised tumours were washed with PBS, dissected into smaller fragments using scalpels, and further dissociated into single cell suspensions using the Miltenyi Tumour Dissociation Kit (#130-096-730) and the GentleMACS Octo dissociator (Miltenyi #130-095-937). The digested tumours were filtered through 70 μM pre-separation filters (Corning), washed with PBS, and then used for flow cytometry.

For each sample, 1×10⁶ cells were treated with mouse Fc Blocking solution (Mouse BD Fc Block #553141) and then stained with a defined panel containing live/dead stain (eBioscience) and seven different labeling antibodies. Anti-CD3 (17A2), anti-CD4 (GK1.5), anti-CD8 (53-6.7), anti-CD45 (30-F11) for surface staining and anti-IL-2 (JES6-5H4), IFNg (XMG1.2) and TNFa (MP6-XT22) for intra-cellular markers

Perm/Wash Buffer (BD Biosciences, #554714) was used to permeabilize and facilitate intracellular staining. All samples were fixed with 2% paraformaldehyde, acquired on a BD FACS Diva, and analyzed by Kaluza (Beckman Coulter).

### Gene Expression Analysis

Gene profile and HBV antigen expression was assessed by NanoString technology which allows direct and digital detection of mRNA molecules in a multiplexed mode. By using pre-designed Mouse_PanCancer_Immune_Profiling_Panel, up to 750 genes can be detected and analysed in a single run.

### RNA Extraction

RNA from frozen tumour tissues were extracted following phenol/chloroform method followed by an additional isolation and purification with Qiagen Rneasy kit. Initially, an optimal sample size piece of tumour was disrupted through high-speed shaking with a Tissue Lyser equipment [2 cycles×3 minutes—25 Hz with tube Magna Lyser green beads Roche Diagnostics ThermoFisher Scientific 50-720-310]. Residual homogenized samples in Tripure Isolation reagent [Sigma-Aldrich/Roche 50 mL 11667157001] were transferred in Eppendorf tubes where aqueous/organic phase separation was performed (Chloroform added to previous Tripure phase—shaking—centrifugation and aqueous phase collection). RNA from the aqueous phase was precipitated by addition of isopropanol. After 30 min incubation and centrifugation, RNA translucent pellet was washed with cold Ethanol 70% then resuspended in nuclease-free water. From there, the Qiagen Rneasy procedure was applied with Dnase treatment (Rneasy Minikit #74106+Rnase Free Dnase Set #79254 Qiagen). Quantification and qualification of final eluted RNA was assessed with UV-visible spectrophotometer [Nanodrop 2000 ThermoFisher].

### NanoString

Total RNA samples were analyzed using the nCounter® PANCancer Mouse Immune Profiling codeset targeting 750 cancer-related mouse genes.

Additional probes for HBV core and HBV surface antigens were added to the commercial panel (Panel Plus nanoString—custom order):
Probe GOI_1: HBv Core, position 139-238 (SEQ NO: 125)
Probe GOI_2: HBv Surface, position 200-299 (SEQ ID NO: 126)

Probeset-target RNA hybridization reactions were performed according to the manufacturer's protocol (CodeSet-Plus/Panel-Plus Reagents with nCounter® XT Gene Expression Assays described in MAN-10023 available on NanoString website).

For each hybridization reaction, 100 ng of total RNA was used.

Purified probeset-target RNA complexes from each reaction were processed and immobilized on nCounter Cartridges using the nCounter® Flex Prep Station, and transcripts were quantified on the Digital Analyzer (GEN 2).

### NanoString Data Processing and Analysis

Raw data were analyzed using nSolver 4.0 software and normalization of the counts was done using a standard method, as follow.

First, a global background was calculated using the determined threshold count value of 20. Then, normalization to internal positive controls was performed to adjust for system variation between lanes. This was achieved by first calculating the positive control factor that was the mean of the geometric means of the positive controls of the different lanes. A normalization factor was calculated for each lane by dividing the positive control factor by the geometric mean of each lane. This value was used as a multiplier for each lane's count values. To determine genes that would perform as good input normalization factors, we first filtered all the genes to include only those whose count values were above the global background in all samples. We then used geNorm algorithm (Vandesompele, 2002) included in nSolver. The top ten most stable genes were selected for reference gene normalization across the samples to control for input variation of the amount of RNA.

Following normalization gene ratios are expressed in Log 2 and used for analysis of differentially expressed genes and gene set analysis through the use of Advanced Analysis 2.0.115 package in nSolver.

Mutlivariate differentially expression analysis was performed through optimal method for each gene using a negative binomial mixture model for low expression probes or a simplified negative binomial model for high expression probes by defining experimental group as predictor. Resulting p-values are adjusted for multiple comparisons by Benjamini-Hochberg adjustment method (Wang, 2016).

Gene set global significance for a covariate is determined by measuring the cumulative evidence for the differential expression of genes in a pathway and calculated as the square root of the mean squared t-statistics of genes while directed global significance takes into account the sign of the t-statistics and measures the tendency of a pathway to have over- or under-expressed genes (Tomfohr, 2005).

### Statistical Methods

Area under the curve (AUC) for [Day 0-Day 9] of tumour volume (mm³) was computed for each mouse and flank. The distributions of AUC responses are assumed to be normal. AUC modelling was performed separately for each flank since (1) selection criteria were applied only to right flank, and (2) AUC of the same mice did not correlate between flanks.

For each flank, a one-way analysis of variance (ANOVA) model was fitted on AUC values by including group (6 levels) as fixed effect. Different variances were assumed in each vaccine group. Means of AUC by group and their 95% CIs as well as mean difference of AUC between groups and their 95% CIs were derived from these models.

Day 0 baseline correction was not performed for the AUC [Day 0-Day 9] model since Day 0 was already included in the response variable computation of the AUC.

As the current study was exploratory, no adjustment for multiplicity was done.

Concerning tumour infiltrating lymphocytes data, for each cell type separately, an analysis of variance (ANOVA) model with group as fixed effects was fitted on the log 10 cells. There was no evidence of interaction between group and flank and no evidence of an effect of the flank. Heterogeneous variances between groups were assumed. This model was used to compute the geometric means per group (over both flanks, with their 95% confidence interval) and the geometric mean ratios between groups (over both flanks, with their 95% confidence intervals). Analysis of tumour infiltrating lymphocytes were also performed separately for each side and are presented in Annex.

Concerning spleen lymphocytes data, for each sample, unspecific signal detected after medium stimulation was removed from the specific signal detected after peptide stimulation (HBc, HBc_F, or HBs). This was performed separately for each of the 7 cytokine combinations (IL-2 and/or IFN-γ and/or TNF-α). Negative values were replaced by 0. Then, the sum of the 7 cytokine combinations was computed. The distributions of HBc, HBc_F or HBs-specific of % of CD4/8+ T-cells responses are assumed to be lognormal. For each cell type and peptide, an analysis of variance (ANOVA) model was fitted on log 10 values by including group (6 levels) as fixed effect. Different variances were assumed in each vaccine group. This model was used to compute the geometric means per group (with their 95% confidence interval) and the geometric mean ratios between groups (with their 95% confidence intervals).

### Study Design

Six groups of mice were treated as detailed in Table 1.

TABLE 1
Study Groups and Dosing Regimen

| GROUP | DAY −43 | DAY −29 | DAY −15 | GROUP | DAY 0 | DAY 3 |
|---|---|---|---|---|---|---|
| GR 1 | 10⁸ vp | HBc-HBs | CT26 cells | GR 1 | 10⁷ pfu | 10⁷ pfu |
| (n = 32) | ChAd155-hli- | antigens/AS01 | engraftment | (n = 16) | MVA-HBV | MVA-HBV |
| | HBV_AG1 | (IM, 50 μl | (right and | | (IT, 50 μl | (IT, 50 μl |
| | (IM, 50 μl | left thigh) | left flanks) | | right flank | right flank |
| | right thigh) | | | | tumour | tumour |
| | HBc-HBs | | | | only) | only) |
| | antigens/AS01 | | | | | |
| | (IM, 50 μl | | | | | |
| | left thigh) | | | | | |
| GR2 | 10⁸ vp | | CT26 cells | GR 2 | 10⁷ pfu | 10⁷ pfu |
| (n = 32) | ChAd155-hli- | | engraftment | (n = 16) | MVA-HBV | MVA-HBV |
| | HBV_AG1 | | (right and | | (IT, 50 μl | (IT, 50 μl |
| | (IM, 50 μl | | left flanks) | | right flank | right flank |
| | right thigh) | | | | tumour | tumour |
| | | | | | only) | only) |
| GR3 | 10⁸ vp | HBc-HBs | CT26 cells | GR3 | PBS (IT) | PBS (IT) |
| (n = 32) | ChAd155-hli- | antigens/AS01 | engraftment | (n = 16) | | |
| | HBV_AG1 | (IM, 50 μl | (right and | | | |
| | (IM, 50 μl | left thigh) | left flanks) | | | |
| | right thigh) | | | | | |
| | HBc-HBs | | | | | |
| | antigens/AS01 | | | | | |
| | (IM, 50 μl | | | | | |
| | left thigh) | | | | | |
| GR4 | PBS (IM, | PBS (IM, | CT26 cells | GR4 | 10⁷ pfu | 10⁷ pfu |
| (n = 32) | right tigh) | right thigh) | engraftment | (n = 16) | MVA-HBV | MVA-HBV |
| | | | (right and | | (IT, 50 μl | (IT, 50 μl |
| | | | left flanks) | | right flank | right flank |
| | | | | | tumour | tumour |
| | | | | | only) | only) |
| GR5 | 10⁸ vp | HBc-HBs | CT26 cells | GR5 | 10⁷ pfu | 10⁷ pfu |
| (n = 32) | ChAd155-hli- | antigens/AS01 | engraftment | (n = 16) | MVA_AG2 | MVA_AG2 |
| | HBV-C_AG1 | (IM, 50 μl | (right and | | (IT, 50 ul | (IT, 50 ul |
| | (IM, 50 μl | left thigh) | left flanks) | | right flank | right flank |
| | right thigh) | | | | tumour) | tumour) |
| | HBc-HBs | | | | | |
| | antigens/AS01 | | | | | |
| | (IM, 50 μl | | | | | |
| | left thigh) | | | | | |
| GR6 | PBS (IM, | PBS (IM, | CT26 cells | GR6 | PBS (IT, | PBS (IT, |
| (n = 32) | right thigh) | right thigh) | engraftment | (n = 16) | right flank | right flank |
| | | | (right and | | only | only) |
| | | | left flanks) | | | |

Because of the constrains of the study design requiring vaccination prior to tumour implantation, the mice were not randomly assigned to treatment groups but rather assigned at the start of the study to respective groups. Therefore, 32 mice/group were initially engrafted with CT26 tumour cells to mitigate for variability of engraftment and tumour development. At the day of MVA dosing, among the 32 animals/group, 16 mice were selected based on their right tumour size (80-120 mm³) and included for further analyses of tumour growth.

### Results

### Tumour Growth

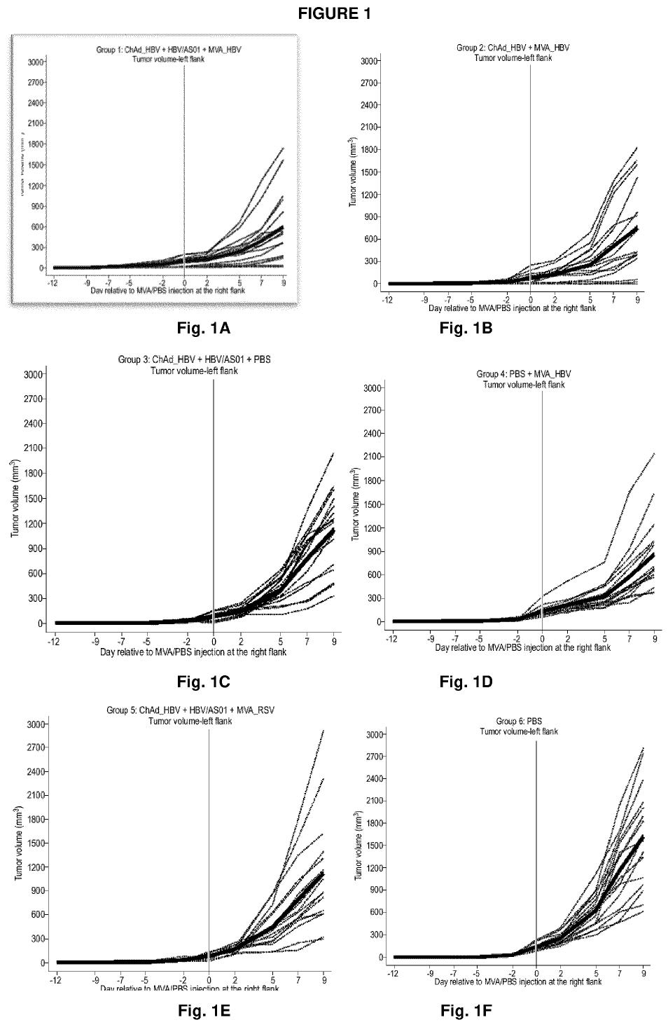

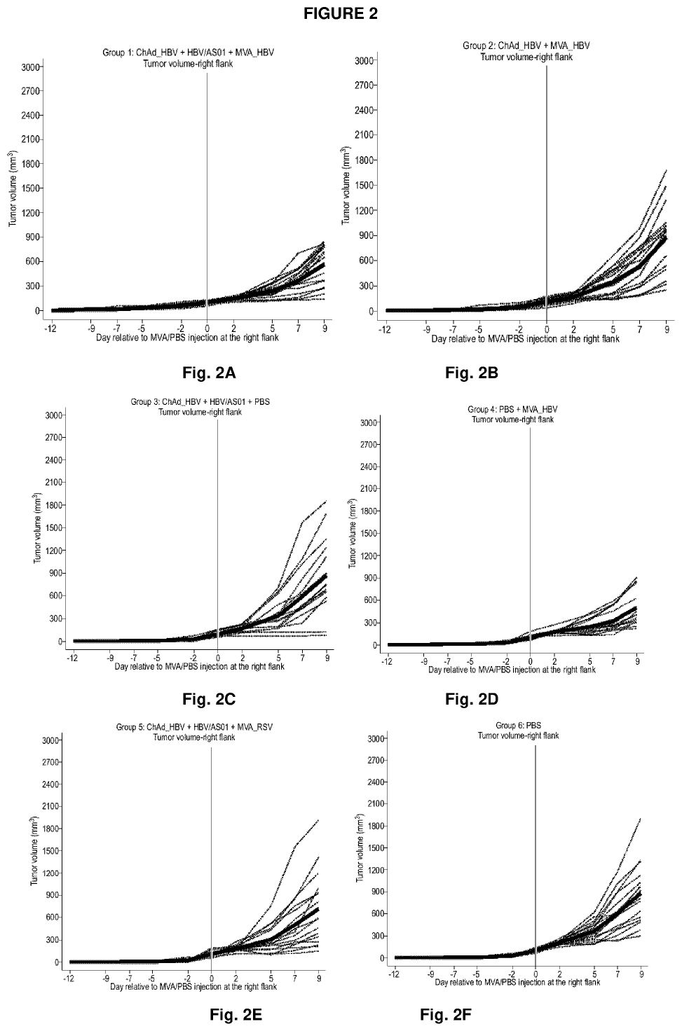

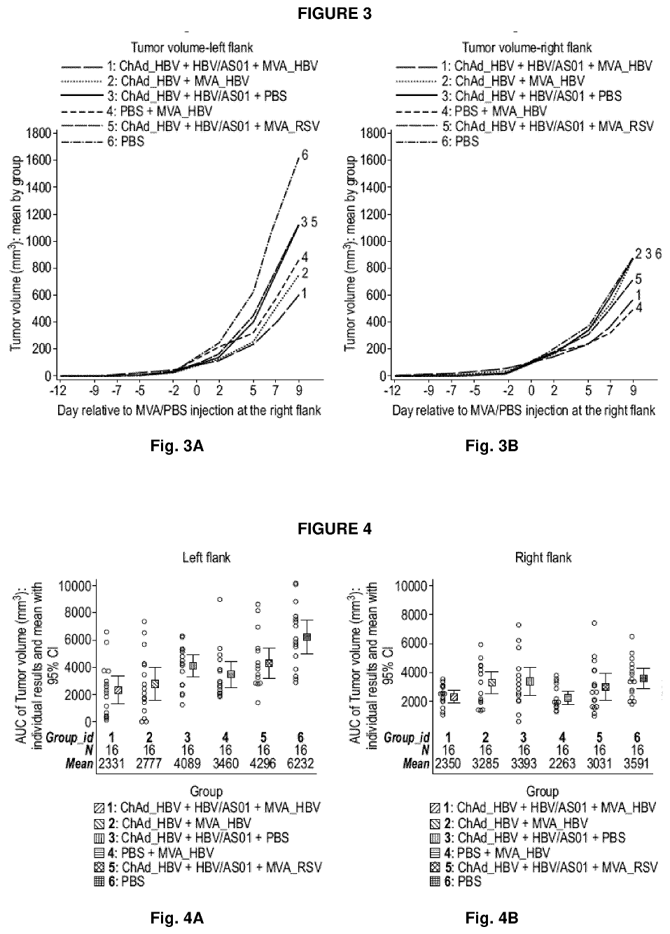

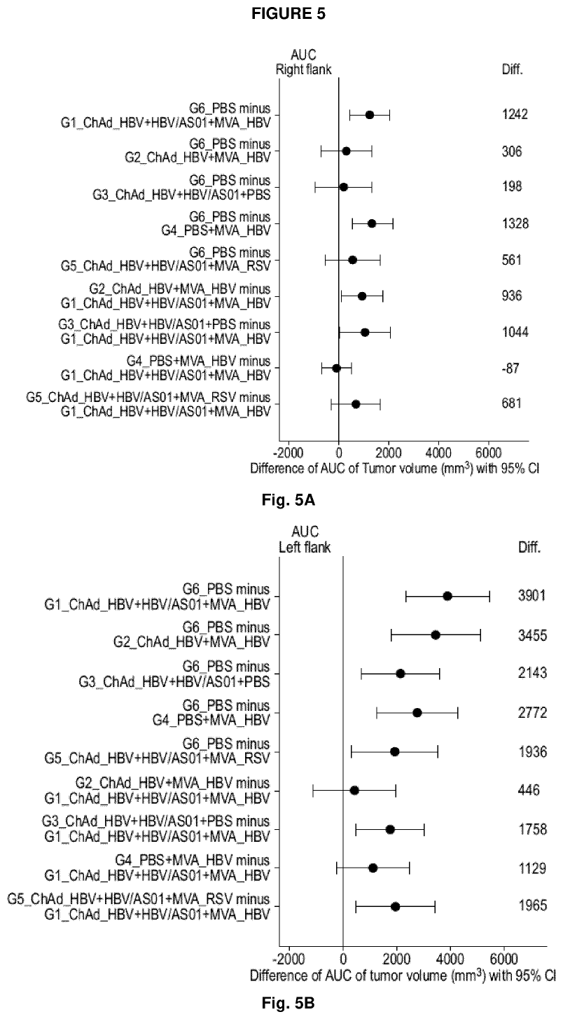

The individual tumour volumes are presented for each group and flank in FIGS. 1A-1F (left flank) and 2A-2F (right flank). Means by group are illustrated by the bold line as in FIGS. 3A and 3B. In order to statistically compare growth curves for the different groups and treatments, AUCs between the different groups were compared. Means of AUC of tumour volume for [Day 0-Day 9] with their 95% CIs by group and flank are presented in FIGS. 4A and 4B. Mean difference of AUC between groups and their 95% CIs are presented for each flank in FIGS. 5A and 5B.

Overall, AUCs of tumour volume seem to be lower at the right than at the left flank. Comparing Group 6 (receiving only PBS: control group) between flanks might suggest that when a needle is inserted in the tumour, tumour volume is reduced. Injection manipulation in the tumour by itself (irrespective of vaccine treatment) might have an impact on tumour volume growth. Nevertheless, comparisons of AUCs show significant differences on both flanks for Groups 1 and 4 versus control Group 6 as suggested by the observation of individual growth curves.

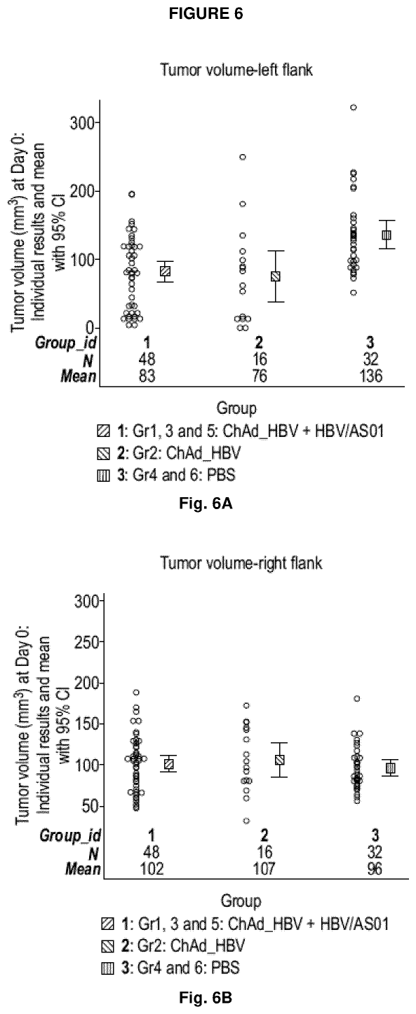

The impact of mice selection at the time of MVA intra-tumour injection was also assessed. FIGS. 6A and 6B shows tumour volume of selected mice at Day 0 just before MVA injection. The selection criteria were based on the right side tumours leading to some tumours on the left side having a volume out of range (below 80 mm³). The range of tumour volumes on the left side was more heterogenous than on the right side making interpretation of tumour growth differences on left side more difficult.

### Immunogenicity

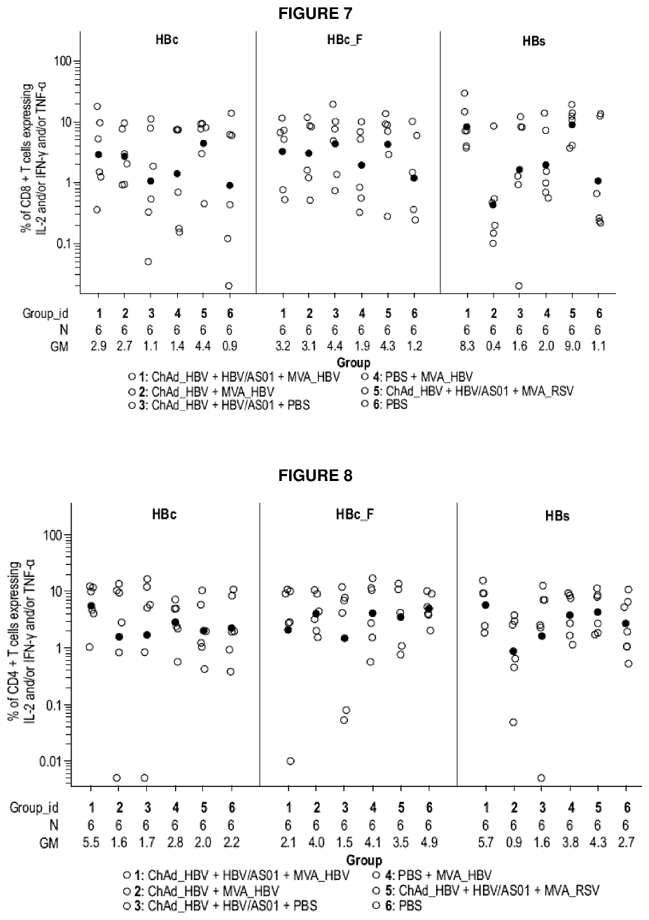

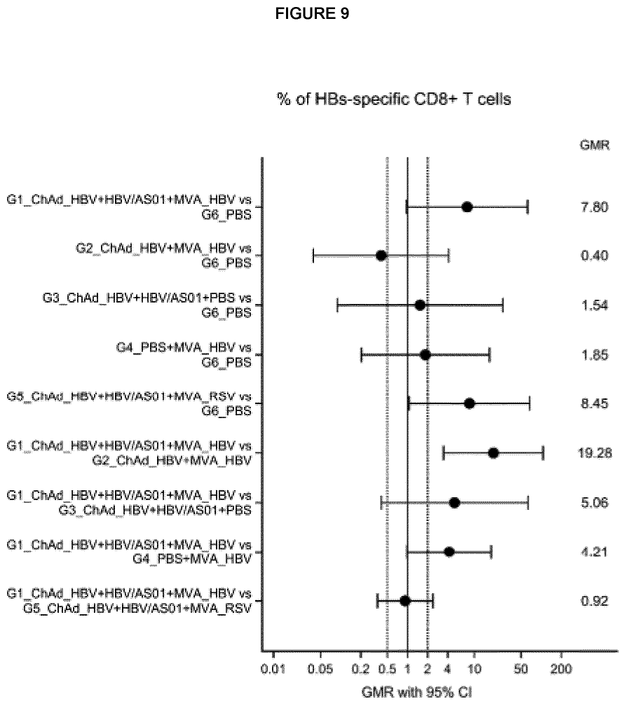

As described in the Study Design section, 6 mice/group were selected for further analysis of immunogenicity, tumour infiltrating lymphocytes and immunogenicity. Immunogenicity elicited by prior vaccination was assessed in terms of frequency of antigen specific CD4+ and CD8+ T cells presenting at least one activation marker (i.e, IFNg, TNFa and IL-2). Results are presented in FIGS. 7 and 8. Statistical analyses are presented in FIG. 9 with Geometric mean ratio and confidence interval being at the limit of significance for Group 1 vs. Group 6 comparisons.

### Tumour Immune Infiltration

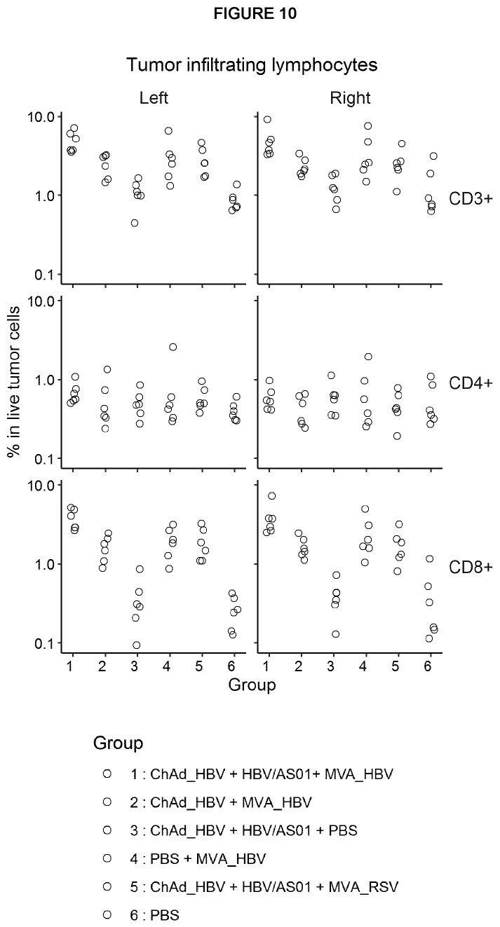

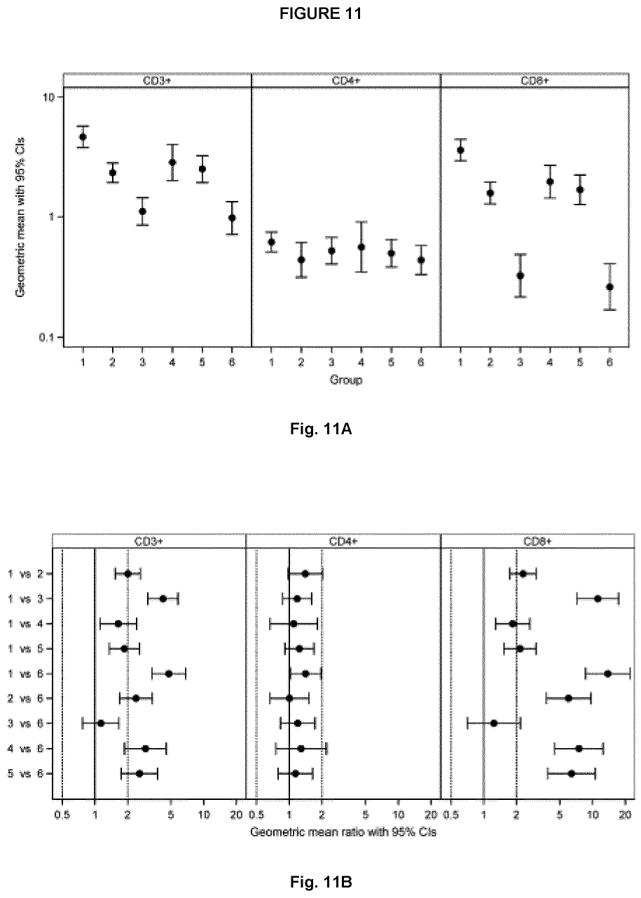

The tumour infiltration by lymphocytes was assessed by the frequency of CD3+, CD4+ and CD8+ T cells within the tumours for each flank after dissociation and flow cytometry analysis. The individual percentage of CD3+, CD4+ and CD8+ T cells among live tumour cells are presented in FIG. 10 for the right and left flank. Percentages from the right and left sides appear similar (no effect of the flank, confirmed by an ANOVA model). In absence of interaction between group and flank effects, the geometric mean percentage per group (overall for both right and left flank taken together) and their 95% confidence interval is presented in FIGS. 11A and 11B and Error! Reference source not found. 2.

TABLE 2
Geometric mean percentage of tumour infiltrating lymphocytes
(CD3+, CD4+, CD8+) and 95% confidence intervals

| | | Geometric | Lower Limit | Upper Limit |
|---|---|---|---|---|
| Cell type | Group | mean | of 95% CI | of 95% CI |
| CD3+ | 1 | 4.66 | 0.58 | 0.76 |
| CD3+ | 2 | 2.32 | 0.29 | 0.45 |
| CD3+ | 3 | 1.10 | −0.07 | 0.16 |
| CD3+ | 4 | 2.84 | 0.30 | 0.61 |
| CD3+ | 5 | 2.50 | 0.28 | 0.51 |
| CD3+ | 6 | 0.98 | −0.15 | 0.12 |
| CD4+ | 1 | 0.62 | −0.29 | −0.13 |
| CD4+ | 2 | 0.44 | −0.50 | −0.21 |
| CD4+ | 3 | 0.52 | −0.39 | −0.17 |
| CD4+ | 4 | 0.56 | −0.46 | −0.04 |
| CD4+ | 5 | 0.50 | −0.42 | −0.19 |
| CD4+ | 6 | 0.44 | −0.48 | −0.24 |
| CD8+ | 1 | 3.61 | 0.47 | 0.65 |
| CD8+ | 2 | 1.57 | 0.11 | 0.29 |
| CD8+ | 3 | 0.32 | −0.67 | −0.31 |
| CD8+ | 4 | 1.96 | 0.16 | 0.43 |
| CD8+ | 5 | 1.68 | 0.10 | 0.35 |
| CD8+ | 6 | 0.26 | −0.77 | −0.39 |

Separate analyses for right and left flank are also presented in Table 3.

TABLE 3
Geometric mean percentage of tumour infiltrating
lymphocytes (CD3+, CD4+, CD8+) and 95%
confidence intervals for right and left side independently

| | | | Geometric | Lower Limit | Upper Limit |
|---|---|---|---|---|---|
| Flank | Cell type | Group | mean | of 95% CI | of 95% CI |
| Left | CD3+ | 1 | 4.74 | 0.54 | 0.81 |
| Left | CD3+ | 2 | 2.37 | 0.25 | 0.50 |
| Left | CD3+ | 3 | 1.02 | −0.16 | 0.18 |
| Left | CD3+ | 4 | 2.69 | 0.20 | 0.66 |
| Left | CD3+ | 5 | 2.66 | 0.26 | 0.59 |
| Left | CD3+ | 6 | 0.85 | −0.26 | 0.12 |
| Left | CD4+ | 1 | 0.67 | −0.30 | −0.06 |
| Left | CD4+ | 2 | 0.48 | −0.53 | −0.11 |
| Left | CD4+ | 3 | 0.48 | −0.48 | −0.15 |
| Left | CD4+ | 4 | 0.56 | −0.56 | 0.06 |
| Left | CD4+ | 5 | 0.57 | −0.41 | −0.08 |
| Left | CD4+ | 6 | 0.40 | −0.58 | −0.23 |
| Left | CD8+ | 1 | 3.65 | 0.43 | 0.70 |
| Left | CD8+ | 2 | 1.55 | 0.05 | 0.32 |
| Left | CD8+ | 3 | 0.30 | −0.79 | −0.27 |
| Left | CD8+ | 4 | 1.81 | 0.06 | 0.46 |
| Left | CD8+ | 5 | 1.77 | 0.06 | 0.43 |
| Left | CD8+ | 6 | 0.24 | −0.91 | −0.34 |
| Right | CD3+ | 1 | 4.59 | 0.53 | 0.80 |
| Right | CD3+ | 2 | 2.28 | 0.24 | 0.48 |
| Right | CD3+ | 3 | 1.20 | −0.09 | 0.25 |
| Right | CD3+ | 4 | 3.01 | 0.25 | 0.71 |
| Right | CD3+ | 5 | 2.36 | 0.20 | 0.54 |
| Right | CD3+ | 6 | 1.12 | −0.15 | 0.24 |
| Right | CD4+ | 1 | 0.57 | −0.36 | −0.12 |
| Right | CD4+ | 2 | 0.40 | −0.61 | −0.18 |
| Right | CD4+ | 3 | 0.56 | −0.41 | −0.08 |
| Right | CD4+ | 4 | 0.56 | −0.56 | 0.06 |
| Right | CD4+ | 5 | 0.44 | −0.52 | −0.20 |
| Right | CD4+ | 6 | 0.48 | −0.49 | −0.14 |
| Right | CD8+ | 1 | 3.57 | 0.42 | 0.69 |
| Right | CD8+ | 2 | 1.60 | 0.07 | 0.34 |
| Right | CD8+ | 3 | 0.35 | −0.71 | −0.19 |
| Right | CD8+ | 4 | 2.12 | 0.12 | 0.53 |
| Right | CD8+ | 5 | 1.60 | 0.02 | 0.39 |
| Right | CD8+ | 6 | 0.29 | −0.83 | −0.26 |

The associated geometric mean ratios between groups and their 95% confidence intervals is presented in FIG. 11 and Table 4.

TABLE 4
Geometric mean ratios between percentage of
tumour infiltrating lymphocytes (CD3+, CD4+, CD8+) and
95% confidence intervals

| | Group | Geometric | Lower Limit | Upper Limit |
|---|---|---|---|---|
| Cell type | comparison | mean ratio | of 95% CI | of 95% CI |
| CD3+ | 1 vs 2 | 2.01 | 0.19 | 0.42 |
| CD3+ | 1 vs 3 | 4.22 | 0.49 | 0.76 |
| CD3+ | 1 vs 4 | 1.64 | 0.05 | 0.38 |
| CD3+ | 1 vs 5 | 1.86 | 0.13 | 0.41 |
| CD3+ | 1 vs 6 | 4.78 | 0.53 | 0.83 |
| CD3+ | 2 vs 3 | 2.10 | 0.19 | 0.46 |
| CD3+ | 2 vs 4 | 0.82 | −0.25 | 0.08 |
| CD3+ | 2 vs 5 | 0.93 | −0.16 | 0.10 |
| CD3+ | 2 vs 6 | 2.38 | 0.23 | 0.53 |
| CD3+ | 3 vs 4 | 0.39 | −0.59 | −0.23 |
| CD3+ | 3 vs 5 | 0.44 | −0.51 | −0.20 |
| CD3+ | 3 vs 6 | 1.13 | −0.11 | 0.22 |
| CD3+ | 4 vs 5 | 1.14 | −0.12 | 0.24 |
| CD3+ | 4 vs 6 | 2.91 | 0.27 | 0.66 |
| CD3+ | 5 vs 6 | 2.56 | 0.24 | 0.58 |
| CD4+ | 1 vs 2 | 1.41 | −0.01 | 0.31 |
| CD4+ | 1 vs 3 | 1.18 | −0.06 | 0.20 |
| CD4+ | 1 vs 4 | 1.10 | −0.18 | 0.26 |
| CD4+ | 1 vs 5 | 1.24 | −0.04 | 0.23 |
| CD4+ | 1 vs 6 | 1.41 | 0.01 | 0.29 |
| CD4+ | 2 vs 3 | 0.84 | −0.25 | 0.10 |
| CD4+ | 2 vs 4 | 0.78 | −0.35 | 0.13 |
| CD4+ | 2 vs 5 | 0.88 | −0.23 | 0.12 |
| CD4+ | 2 vs 6 | 1.01 | −0.17 | 0.18 |
| CD4+ | 3 vs 4 | 0.93 | −0.26 | 0.20 |
| CD4+ | 3 vs 5 | 1.05 | −0.13 | 0.17 |
| CD4+ | 3 vs 6 | 1.20 | −0.08 | 0.23 |
| CD4+ | 4 vs 5 | 1.13 | −0.18 | 0.28 |
| CD4+ | 4 vs 6 | 1.28 | −0.12 | 0.34 |
| CD4+ | 5 vs 6 | 1.14 | −0.10 | 0.21 |
| CD8+ | 1 vs 2 | 2.29 | 0.24 | 0.48 |
| CD8+ | 1 vs 3 | 11.15 | 0.86 | 1.24 |
| CD8+ | 1 vs 4 | 1.84 | 0.11 | 0.42 |
| CD8+ | 1 vs 5 | 2.15 | 0.19 | 0.48 |
| CD8+ | 1 vs 6 | 13.81 | 0.94 | 1.34 |
| CD8+ | 2 vs 3 | 4.86 | 0.50 | 0.88 |
| CD8+ | 2 vs 4 | 0.80 | −0.25 | 0.06 |
| CD8+ | 2 vs 5 | 0.94 | −0.17 | 0.12 |
| CD8+ | 2 vs 6 | 6.02 | 0.58 | 0.98 |
| CD8+ | 3 vs 4 | 0.17 | −0.99 | −0.57 |
| CD8+ | 3 vs 5 | 0.19 | −0.92 | −0.51 |
| CD8+ | 3 vs 6 | 1.24 | −0.15 | 0.34 |
| CD8+ | 4 vs 5 | 1.17 | −0.11 | 0.24 |
| CD8+ | 4 vs 6 | 7.49 | 0.65 | 1.10 |
| CD8+ | 5 vs 6 | 6.41 | 0.59 | 1.02 |

Overall, the results for tumour infiltrating lymphocytes show that group 1 (Pre-vaccination followed by intra-tumour MVA) shows percentages of CD3+ and CD8+ T cells that are higher than all other groups.

### Tumour Related Gene Expression

To obtain a more comprehensive view of immune functions within solid tumours at the time of tumour collection (Day 9 post MVA IT injection), RNA expression of over 750 immunologically relevant genes in tumour samples (right & left flank separately) was examined using the Nanostring PanCancer Immune profiling panel. This large gene panel can be divided into specific gene sets relevant to different immune cell functions, such as immune cell types (NK cell functions, T-cell functions, macrophage functions, etc.) as well as immunology/inflammation pathways (interferon, cytokines & receptors, etc.).

TABLE 5
nCounter ® PANCancer Mouse Immune
Profiling Gene sets summary

| Gene set | # of Genes |
|---|---|
| Adaptive | 112 |
| Adhesion | 65 |
| Antigen Processing | 34 |
| Apoptosis | 111 |
| Basic Cell Functions | 56 |
| B-Cell Functions | 85 |
| Cancer Progression | 57 |
| CD molecules | 212 |
| Cell Cycle | 36 |
| Chemokines & Receptors | 84 |
| Complement Pathway | 32 |
| Cytokines & Receptors | 191 |
| Dendritic Cell Functions | 17 |
| Humoral | 57 |
| Inflammation | 152 |
| Innate | 225 |
| Interferon | 40 |
| Interleukins | 128 |
| Leukocyte Functions | 41 |
| Macrophage Functions | 45 |
| Mast Cell Functions | 4 |
| MHC | 33 |
| Microglial Functions | 5 |
| NK Cell Functions | 53 |
| Pathogen Response | 29 |
| Senescence | 22 |
| T-Cell Functions | 200 |
| TLR | 26 |
| TNF Superfamily | 34 |
| Transporter Functions | 99 |

The list of genes included in each of the nCounter® PANCancer Mouse Immune Profiling gene set is detailed hereafter:
Adaptive: C1qbp, C3ar1, Camp, Ccl11, Ccl12, Ccl1, Ccl24, Ccl25, Ccl26, Ccl3, Ccl4, Ccl5, Ccl7, Ccl8, Ccr1, Ccr2, Ccr4, Ccr5, Ccr6, Ccr7, Ccr8, Ccr12, Cd28, Cd40lg, Cd40, Cd4, Cd80, Cd86, Cd8a, Cd97, Cklf, Cma1, Creb5, Crp, Csf2, Cxcl10, Cxcl13, Cxcl14, Cxcl1, Cxcl2, Cxcl3, Cxcl9, Cxcr1, Cxcr2, Cxcr3, Fasl, Fcer1a, Fcer2a, Foxp3, Fpr2, Gata3, H2-Q10, Hmgb1, Icam1, Ido1, Ifna2, Ifnar1, Ifnb1, Ifng, Ifngr1, Il10, Il13, Il17a, Il18, Il1a, Il1b, Il1r1, Il22, Il23a, Il24, Il25, Il2, Il2ra, Il4, Il5, Il6, Il6st, Il9, Irf3, Irf7, Itgam, Itk, Jak2, Jam3, Klrb1, Lta, Mapk8, Mbl2, Ms4a2, Mx1, Nfatc4, Nfkb1, Nod2, Nos2, Pnma1, Rag1, Rorc, S100a8, Sele, Slc11a1, Stat1, Stat3, Stat4, Stat6, Tbx21, Thbs1, Tlr4, Tlr6, Tnf, Tnfrsf11a, Txk, Xcr1.
Adhesion: Ada, Alcam, Amica1, Angpt2, Cd164, Cd22, Cd33, Cd63, Cd6, Cd84, Cd96, Cd97, Cd9, Cdh5, Col3a1, Csf3r, Cyfip2, Fn1, Glycam1, Icam2, Icam4, Irf2, Itga1, Itga2b, Itga2, Itga4, Itga5, Itga6, Itgae, Itgal, Itgax, Itgb1, Itgb4, Kdr, Klra4, Klra5, Klra6, Klra7, Ly9, Lyve1, Map2k1, Mcam, Mertk, Mfge8, Mmp9, Msln, Ncam1, Nrp1, Pecam1, Plau, Pvrl2, Saa1, Sell, Selplg, Siglec1, Spink5, Spn, Spp1, Tek, Thy1, Tnfrsf12a, Vcam1, Vwf.
Antigen Processing: Ccr7, Cd1d1, Cd1d2, Cd74, Fcer1g, Fcgr1, Fcgr2b, Fcgr3, H2-Aa, H2-Ab1, H2-D1, H2-DMa, H2-DMb1, H2-DMb2, H2-Ea-ps, H2-Eb1, H2-K1, H2-M3, H2-Ob, H2-Q1, H2-Q2, H2-T23, Icam1, Mr1, Nod1, Nod2, Psmb8, Psmb9, Relb, Tap1, Tap2, Tapbp.
Apoptosis: Abl1, Adora2a, Angpt1, Apoe, App, Atg5, Atg7, Atm, Bax, Bcl2l1, Bid, Birc5, Btk, C6, C9, Casp3, Casp8, Cd38, Cd3g, Cd59b, Cd5, Cdk1, Clec5a, Clu, Ctsh, Cyld, Dusp6, Egfr, Egr3, Ep300, Ets1, Fadd, Gpi1, Gzma, Gzmb, Hif1a, Hmgb1, Ifih1, Igf2r, Ikbke, Il19, Il24, Il3, Inpp5d, Jun, Lck, Lcn2, Litaf, Lrp1, Ltbr, Ltk, Map2k4, Map3k1, Map3k5, Map3k7, Mapk1, Mapk3, Mef2c, Muc1, Myc, Nefl, Nlrp3, Nos2, Osm, Pdcd1, Pik3cg, Plaur, Pml, Prkcd, Psen1, Ptgs2, Pycard, Rps6, Runx3, S100b, Tcf7, Tgfb2, Tgfb3, Tgfbr1, Tgfbr2, Tmem173, Tnfaip3, Tnfrsf10b, Tnfrsf11b, Tnfrsf18, Tnfrsf8, Tnfsf10, Tnfsf12, Tnfsf14, Tnfsf15, Traf2, Traf3, Trp53, Twist1, Txnip, Vegfa, Xaf1.
B-Cell Functions: Bcl10, Bcl2, Bcl6, Blk, Blnk, Bmi1, Btla, Card11, Cd19, Cd200r1, Cd27, Cd28, Cd37, Cd40lg, Cd40, Cd4, Cd69, Cd70, Cd74, Cd79a, Cd79b, Cd81, Cd83, Cd86, Cdkn1a, Cr2, Ctla4, Cxcl13, Cxcr5, Dpp4, Fas, Fcer2a, Fcgr2b, Flt3, Foxp3, Gpr183, Icosl, Ikbkb, Ikbkg, Ikzf1, Il10, Il11, Il13, Il13ra1, Il1r2, Il21, Il2ra, Il2rg, Il4, Il5, Il6, Il7, Il7r, Jak3, Lyn, Mif, Ms4a1, Nt5e, Prdm1, Ptprc, Sh2b2, Stat5b, Syk, Tgfb1, Ticam1, Tirap, Tnfrsf13b, Tnfrsf13c, Tnfrsf4, Tnfsf13b, Tnfsf4.
Basic cell Functions: Aicda, Arg1, Arg2, Chil3, Chit1, Cmpk2, Ctsg, Ctsl, Ddx60, Dock9, Dusp4, Emr1, Epsti1, Ewsr1, F12, F13a1, Gbp2b, Hamp, Hcst, Herc6, Hsd11b1, Isg15, Isg20, Map2k2, Mapk11, Mpo, Mpped1, Ncf4, Notch1, Oas2, Oas3, Oasl1, Pdgfc, Pla2g1b, PIa2g6, Pmch, Pou2af1, Prg2, Psmb7, Raet1c, Reps1, Rrad, Rsad2, St6gal1, Stat2, Tab1, Tank, Timd4, Trem2, Ubc, Usp18, Usp9y, Ythdf2, Zfp13.
Cancer Progression: Akt3, C3ar1, C3, Ccl11, Ccl5, Ccl7, Ccl8, Ccr2, Ccr3, Cd163, Cd34, Cd36, Cd44, Cd46, Cdh1, Ceacam1, Cfp, Cma1, Col1a1, Col4a1, Crebbp, Csf2rb, Cspg4, Dll4, Erbb2, Fap, Hspb2, Psma2, Sele, Smad2, Smad3, Smad4, Smn1, Snai1, Tdo2, Tie1, Vegfc, Vhl, Vim.
CD Molecules: Abcb1a, Bst1, Bst2, Ccr1, Ccr2, Ccr3, Ccr4, Ccr5, Ccr6, Ccr7, Ccr9, Cd14, Cd160, Cd180, Cd1d1, Cd1d2, Cd200, Cd207, Cd209e, Cd244, Cd247, Cd274, Cd276, Cd28, Cd2, Cd3d, Cd3eap, Cd3e, Cd40lg, Cd40, Cd47, Cd48, Cd4, Cd53, Cd55, Cd68, Cd74, Cd7, Cd83, Cd86, Cd8b1, Cd97, Cd99, Cr2, Csf1r, Ctsw, Cxcr1, Cxcr2, Cxcr3, Cxcr4, Cxcr6, Eng, Entpd1, Epcam, Fas, Fcer2a, Fcgr1, Fcgr2b, Fcgr4, Icam1, Icos, Ifitm1, Ifngr1, Igf1r, Igll1, Il10ra, Il12rb1, Il13ra1, Il13ra2, Il15ra, Il17ra, Il18r1, Il18rap, Il1r1, Il21r, Il2ra, Il2rb, Il2rg, Il3ra, Il4ra, Il5ra, Il6ra, Il6st, Itgam, Itgb2, Itgb3, Kit, Klrc1, Klrc2, Klrk1, Lag3, Lamp1, Lamp2, Lamp3, Lilra5, Lrrn3, Mme, Mrc1, Msr1, Mst1r, Ncr1, Pdcd1lg2, Pdgfrb, Psmd7, Ptgdr2, Pvr, Sele, Slamf1, Slamf6, Slamf7, Tfrc, Thbd, Tlr1, Tlr2, Tlr3, Tlr4, Tlr6, Tlr8, Tlr9, Tnfrsf11a, Tnfrsf14, Tnfrsf17, Tnfrsf1a, Tnfrsf1b, Tnfrsf4, Tnfrsf9, Tnfsf11, Tnfsf13b, Tnfsf13, Tnfsf14, Tnfsf4, Tnfsf8, Trem1.
Cell Cycle: Anp32b, Anxa1, Ccnd3, Cxcl15, Il12a, Il12b, Map2k1, Nfatc1, Pin1, Prkce, Smpd3, Stat5b, Tal1, Tgfb1.
Chemokines & Receptors: C5ar1, Ccl11, Ccl12, Ccl17, Ccl19, Ccl20, Ccl21a, Ccl22, Ccl25, Ccl28, Ccl2, Ccl3, Ccl4, Ccl5, Ccl6, Ccl7, Ccl8, Ccl9, Ccr1, Ccr2, Ccr3, Ccr4, Ccr5, Ccr6, Ccr7, Cklf, Cmklr1, Csf1r, Cx3cl1, Cx3cr1, Cxcl10, Cxcl11, Cxcl12, Cxcl13, Cxcl15, Cxcl16, Cxcl1, Cxcl2, Cxcl5, Cxcl9, Cxcr1, Cxcr2, Cxcr3, Cxcr4, Elane, Hc, Ifng, Il16, Il18, Il1b, Il1rl1, Il22ra1, Il4, Il4ra, Il6, Ilf3, Itch, Jak1, Lbp, Myd88, Ppbp, Sigirr, Tgfb1, Ticam1, Tirap, Tlr3, Tlr4, Tlr7, Tlr9, Tnf, Tnfsf4, Xcl1.
Complement Pathway: A2m, C1qa, C1qb, C1ra, C1s1, C2, C3ar1, C3, C4b, C7, C8a, C8b, C8g, Cfb, Cfd, Cfh, Cfi, Cr2, Crp, Hc, Masp1, Masp2, Mbl2, Serping1.
Cytokines & Receptors: Casp1, Ccl11, Ccl12, Ccl19, Ccl25, Ccl27a, Ccl2, Ccl3, Ccl4, Ccl7, Ccl8, Ccr1, Ccr2, Ccr3, Ccr5, Cd14, Cd40lg, Cklf, Clec4n, Csf1, Csf1r, Csf2, Csf3, Cxcl10, Cxcl12, Cxcl13, Cxcl15, Cxcl1, Cxcl2, Cxcl9, Cxcr1, Cxcr2, Cxcr3, Cxcr4, Ebi3, Elane, F2rl1, Fasl, Flt3l, Foxp3, Hc, Ido1, Ifna1, Ifna4, Ifnar2, Ifnb1, Ifng, Ifngr1, Il10, Il11ra1, Il12a, Il12b, Il12rb1, Il12rb2, Il13, Il13ra1, Il15, Il17a, Il17b, Il17f, Il17rb, Il18, Il18rap, Il1a, Il1r1, Il1rapl2, Il1rap, Il1rl1, Il1rl2, Il1rn, Il21, Il22ra2, Il23a, Il23r, Il24, Il25, Il27, Il2rg, Il34, Il4, Il4ra, Il5, Il6, Il6st, Il7, Il9, Ikak1, Irak2, Irak3, Irak4, Irf3, Jak2, Jak3, Lif, Lta, Ltb, Lyn, Mif, Myd88, Nfatc2, Nfkb1, Nod2, Pparg, Prkce, Rel, Socs1, Tgfb1, Ticam2, Tnf, Tnfrsf11a, Tnfrsf1a, Tnfrsf4, Tnfsf11, Tnfsf13b, Tnfsf14, Tnfsf18, Tnfsf4, Traf2, Traf6, Xcl1.
Dendritic Cell Functions: Ccl19, Ccl5, Ccr1, Ccr2, Ccr5, Cd40lg, Cd40, Cd83, Cd86, Cr2, Cxcr1, Cxcr4, 1110, Lyn, Tgfb1.
Humoral: Aire, Ccl12, Ccl2, Ccl3, Ccl7, Ccr2, Ccr6, Ccr7, Cd28, Cd40, Cd83, Crp, Cxcl13, Fcer2a, Foxj1, Ifnb1, Ifng, Il10, Il1b, Il6, Il7, Itgb2, Lta, Ltf, Ly86, Ly96, Mbl2, Mnx1, Nfkb1, Nod2, Pax5, Pou2f2, Psmb10, Sh2d1a, Tfe3, Tfeb, Tnf.
Inflammation: Axl, C3ar1, C3, Ccl11, Ccl12, Ccl19, Ccl25, Ccl2, Ccl3, Ccl4, Ccl5, Ccl7, Ccl8, Ccr1, Ccr2, Ccr3, Ccr4, Ccr7, Cd14, Cd28, Cd40lg, Cd40, Cd97, Cebpb, Cklf, Clec7a, Cma1, Crp, Csf1, Cxcl10, Cxcl13, Cxcl15, Cxcl1, Cxcl2, Cxcl9, Cxcr1, Cxcr2, Cxcr4, Elane, Fasl, Fas, Fcer2a, Fcgr2b, Fos, Foxp3, Hck, Hc, Hmgb1, Ido1, Il10, Il13, Il17a, Il18, Il1a, Il1b, Il1r1, Il1rl1, Il23r, Il24, Il25, Il27, Il2ra, Il4, Il4ra, Il6, Il6st, Il9, Itgb2, Lbp, Lta, Lyn, Mapkapk2, Mefv, Mif, Myd88, Nfkb1, Nos2, Pik3cd, Pparg, Ripk2, Sbno2, Sele, Tgfb1, Ticam1, Ticam2, Tirap, Tlr1, Tlr2, Tlr3, Tlr4, Tlr5, Tlr6, Tlr7, Tlr8, Tlr9, Tnf, Tnfrsf1a, Tnfrsf4, Tnfsf4, Tollip, Xcl1.
Innate: Abca1, Abcg1, Aft1, Atf2, Atg12, C3, Card9, Ccl2, Ccl5, Ccr1, Ccr3, Ccr6, Cd14, Cd1d1, Cd1d2, Cd4, Cd74, Cd97, Chuk, Clec4a2, Colec12, Cr2, Creb1, Crp, Csf1, Csf1r, Csf2, Ctss, Cxcl10, Cxcl2, Cxcl9, Cxcr2, Cxcr3, Cybb, Ddx58, Defb1, Dmbt1, Ecsit, Elk1, Fcgr1, Gzmk, Gzmm, Hc, Hmgb1, Ifitm2, Ifnb1, Ifngr1, Il18rap, Il1a, Il1b, Il1r1, Il1rl1, Il23a, Il23r, Il27, Il4, Irf3, Irgm2, Itgam, Jak2, Jak3, Klrg1, Lbp, Lgais3, Lyn, Map2k1, Map4k2, Mapk14, Marco, Mays, Mbl2, Mif, Mx2, Myd88, Nfkb1, Nfkb2, Nfkbia, Nlrc5, Nod2, Pparg, Rela, Syk, Tbk1, Ticam1, Ticam2, Tirap, Tlr1, Tlr2, Tlr3, Tlr4, Tlr6, Tlr7, Tlr8, Tlr9, Traf2, Traf6, Tyk2, Xcl1, Zbp1.
Interferon: Ccr7, Ciita, Eomes, Gbp5, H60a, Ifi27, Ifi35, Ifi441, Ifi44, Ifit1, Ifit2, Ifit3, Ifngr1, Ifnl2, Irf8, Nos2, Sh2d1b1, Ulbp1.
Interleukins: Ccl2, Ccr2, Ccr7, Cd1d1, Cd1d2, Cd28, Cd40lg, Cd83, Cma1, Csf2, Cxcl15, Cxcr1, Cxcr2, Egr1, Elane, Fcgr2b, Foxp3, Gfi1, Ido1, Ifng, Il10, Il12a, Il12b, Il2rb1, Il13, Il13ra1, Il17a, Il18, Il18rap, Il1a, Il1b, Il1r1, Il1rl1, Il21, Il23a, Il23r, Il24, Il25, Il27, Il2rg, Il4, Il5, Il6, Il6st, Il7, Il9, Irf1, Irf4, Jak2, Jak3, Lag3, Myd88, Nfkb1, Nod2, Sele, Stat5b, Syk, Ticam1, Ticam2, Tigit, Tirap, Tlr1, Tlr3, Tlr4, Tlr6, Tlr7, Tlr8, Tlr9, Tnfrsf11a, Tnfrsf1a, Tnfsf4, Traf2, Traf6.
Leukocyte Functions: Ccl19, Ccl25, Ccl4, Ccr1, Ccr7, Cklf, Cxcl10, Cxcl12, Cxcl2, Elane, Fut7, Hc, Icam1, Il23r, Itgam, Itgb2, Lbp, Psen2, Sele, Syk, Tlr2.
Macrophage Functions: C3ar1, Ccl2, Ccl5, Ccr2, Ccr5, Ccr7, Cd1d1, Cklf, Crp, Csf1, Csf1r, Csf2, Fcer2a, Hc, Il13, Il18, Il1b, Il1rl1, Il23a, Il4, Il4ra, Lbp, Pparg, Prkce, Rora, Syk, Tlr1.
MHC: Cd1d1, Cd40lg, Cd74, Fcgr1, Fcgr2b, Il10, Lag3.
Microglial Functions: Nod2, Tlr6, Tlr7.
NK Cell Functions: Ccl2, Ccl3, Ccl4, Ccl5, Ccl7, Il12a, Il12b, Il12rb1, Il21, Il23a, Itgb2, Klra15, Klra17, Klra1, Klra20, Klra21, Klra27, Klra2, Klra3, Klrb1c, Klrd1, Lag3, Mill2, Stat5b.
Pathogen Response: Ccl2, Cd14, Hmgb1, Ifnb1, Ifng, Il10, Il12a, Il1b, Il6, Irf3, Lta, Ticam1, Tlr3, Tlr4, Tlr6, Tlr7, Tlr8, Tnf, Tnfrsf1a.
Senescence: Hras, Ifng, Irf3, Irf5, Map2k1, Nfkb1, Serpinb2, Tgfb1.
T-Cell Functions: Ccl1, Ccl19, Ccl2, Ccl3, Ccl5, Ccl7, Ccr2, Ccr3, Ccr4, Ccr5, Ccr6, Ccr7, Cd1d1, Cd1d2, Cd28, Cd40lg, Cd40, Cd4, Cd74, Cd83, Cd86, Cma1, Csf2, Cxcl12, Cxcl13, Cxcr3, Cxcr4, Fasl, Fas, Foxp3, Gpr44, Havcr2, Icam1, Ido1, Ifnb1, Ifng, Ikzf2, Il10, Il12a, Il12b, Il12rb1, Il13, Il13ra1, Il17a, Il18, Il18rap, Il1b, Il1r1, Il21, Il23a, Il25, Il27, Il2ra, Il2rg, Il4, Il4ra, Il5, Il6, Il6st, Il7, Il9, Itgam, Itgb2, Jak2, Jak3, Lag3, Lcp1, Maf, Nfkb1, Nos2, Socs3, Stat5b, Syk, Tgfb1, Tlr4, Tlr6, Tmed1, Tnf, Tnfrsf4, Tnfsf11, Tnfsf13b, Tnfsf14, Tnfsf4, Traf2, Traf6, Xcl1, Yy1, Zap70.
TLR: Cd86, Irf3, Myd88, Prkce, Ticam1, Ticam2, Tirap, Tlr1, Tlr2, Tlr3, Tlr4, Tlr6, Tlr7, Tlr8, Tlr9, Traf6.
TNF Superfamily: Cd40lg, Cd40, Fasl, Fas, Lta, Tnf, Tnfrsf11a, Tnfrsf1a, Tnfrsf4, Tnfsf11, Tnfsf13b, Tnfsf14, Tnfsf4.
Transporter Functions: Ambp, Atg10, Atg16l1, C3, Ccl3, Ccl4, Ccl5, Ccr1, Ccr5, Cd14, Cmah, Crp, Csf1, Csf2, Cxcl12, Cxcl1, Cxcr4, Fas, Fcgr1, Fcgr2b, Fez1, Fyn, Icam1, Ifng, Il13, Il1b, Il1rl1, Il4, Itgam, Itgb2, Lbp, Lyn, Lyz2, Map2k1, Mbl2, Mif, Myd88, Nup107, Pparg, Prkce, Slc7a11, Syk, Syt17, Ticam1, Tlr3, Tlr9, Tnf, Tnfsf11.

Differentially expressed genes (DEGs) between Group 6 (Control group) and all other Groups at the level of right and left flank tumours separately was looked at first. At the right flank, Group 1 had 108 DEGs over 756 (corrected p val. <0.05) when compared to Group 6. At the left flank, Group 1 showed 76 DEGs, Group 2 showed 3 DEGs and Group 5 only 1 DEG when compared to Group 6.

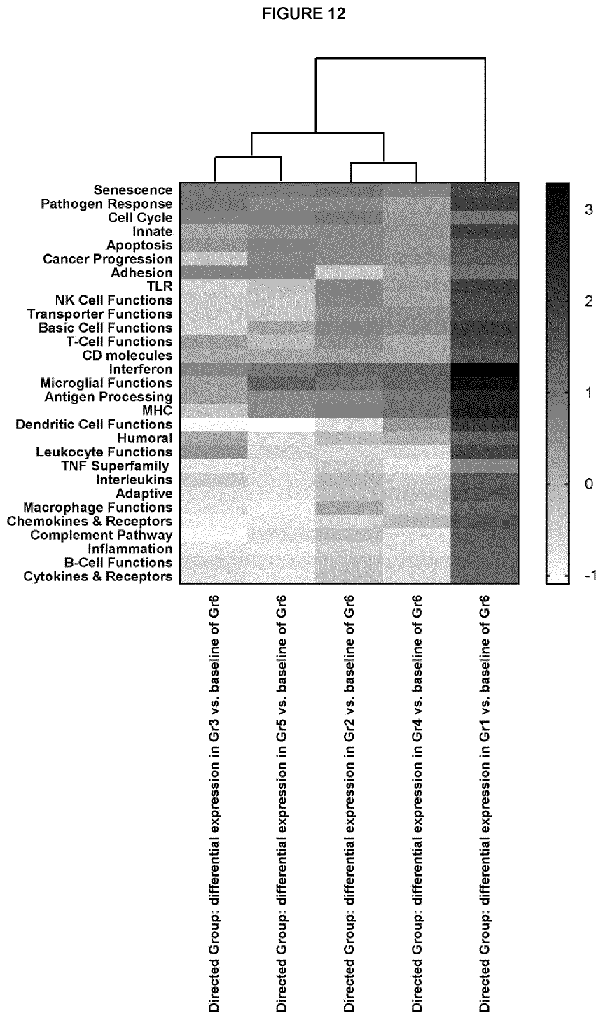

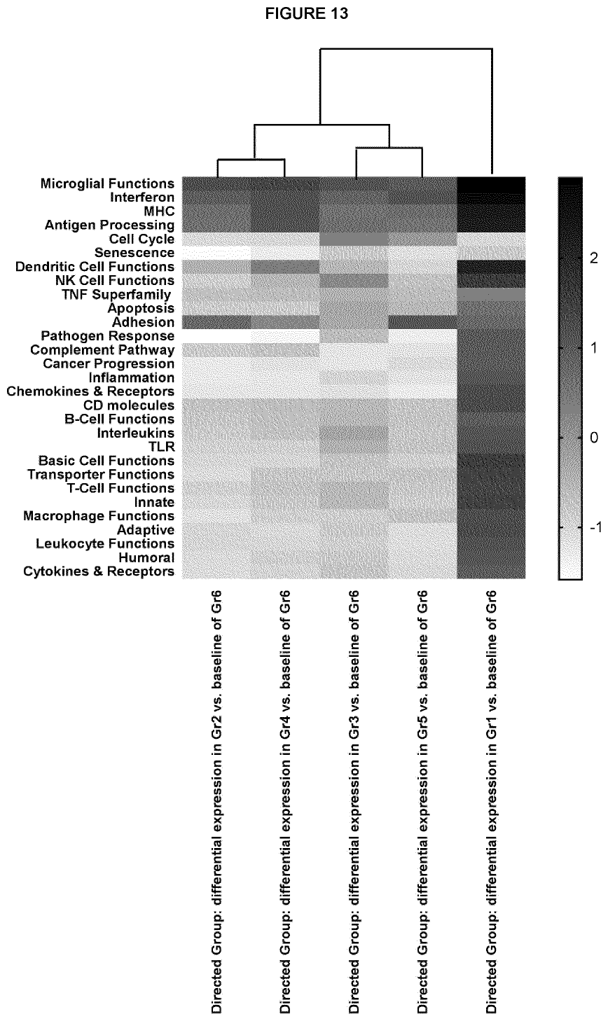

DEG were used to perform gene set enrichment analysis (as described in the Material and Methods section). The most differentially expressed genes for each gene set were identified, and the extent of differential expression in each gene set was summarized in a heatmap displaying each sample directed (up- or down-regulated) global significance scores. As shown in FIG. 12 and Table 6, at the right flank tumour, results indicate a different pathway enrichment profile for Group 1, as illustrated by the hierarchical clustering analysis and the resulting dendrogram. At the left flank tumour, as shown in FIG. 13 and Table 7, similar results are observed with Group 1 showing a different pathway enrichment profiles compared to all other Groups.

The differentially expressed genes were then computed for all groups compared to Group 1. Analysis of differentially expressed genes of all Groups compared to Group 1 resulted in 4 genes being statistically different in all comparisons at the right flank tumours: Cd8b1, Ddx58, Hif1a, Jak2 illustrating a specific modulation of these genes in Group 1 compared to all other treatments.

Gene set enrichment analysis was then performed, as above, and the enrichment scores are presented in Table 8. These scores illustrate the results of pathway enrichment when Group 1 is used as baseline for comparison.

TABLE 6
Directed Global Significance scores for gene set
enrichment analysis when Group 6 is used as baseline
for comparison (Right Tumours)
RIGHT TUMOUR

| | DIRECTED GLOBAL SIGNIFICANCE SCORE | | | | |
|---|---|---|---|---|---|
| | Gr1 vs. | Gr2 vs. | Gr3 vs. | Gr4 vs. | Gr5 vs. |
| | baseline | baseline | baseline | baseline | baseline |
| | of Gr6 | of Gr6 | of Gr6 | of Gr6 | of Gr6 |
| Adaptive | 1.548 | −0.22 | −0.67 | −0.316 | −0.772 |
| Adhesion | 1.076 | −0.325 | 0.748 | 0.21 | 0.742 |
| Antigen | 2.559 | 0.664 | 0.243 | 0.971 | 0.594 |
| Processing | | | | | |
| Apoptosis | 1.464 | 0.612 | 0.439 | 0.386 | 0.758 |
| B-Cell | 1.267 | −0.325 | −0.579 | −0.544 | −0.725 |
| Functions | | | | | |
| Basic Cell | 1.992 | 0.703 | −0.456 | 0.548 | 0.209 |
| Functions | | | | | |
| Cancer | 1.431 | 0.608 | −0.283 | 0.468 | 0.678 |
| Progression | | | | | |
| CD | 1.543 | 0.318 | 0.138 | 0.21 | 0.182 |
| molecules | | | | | |
| Cell Cycle | 1.014 | 0.878 | 0.765 | 0.375 | 0.791 |
| Chemokines | 1.514 | −0.504 | −0.912 | −0.177 | −0.829 |
| & Receptors | | | | | |
| Complement | 1.362 | −0.262 | −1.018 | −0.692 | −0.695 |
| Pathway | | | | | |
| Cytokines & | 1.246 | −0.296 | −0.701 | −0.588 | −0.816 |
| Receptors | | | | | |
| Dendritic Cell | 2.056 | −0.662 | −1.043 | 0.396 | −1.089 |
| Functions | | | | | |
| Humoral | 1.347 | −0.167 | 0.157 | 0.036 | −0.723 |
| Inflammation | 1.305 | −0.471 | −0.799 | −0.685 | −0.85 |
| Innate | 1.933 | 0.626 | 0.244 | 0.457 | 0.524 |
| Interferon | 3.29 | 1.225 | 0.682 | 1.269 | 0.948 |
| Interleukins | 1.439 | −0.141 | −0.43 | −0.47 | −0.681 |
| Leukocyte | 1.775 | −0.561 | 0.409 | −0.574 | −0.561 |
| Functions | | | | | |
| Macrophage | 1.256 | 0.137 | −0.751 | −0.431 | −0.93 |
| Functions | | | | | |
| MHC | 2.63 | 0.799 | −0.301 | 1.009 | 0.564 |
| Microglial | 3.057 | 1.009 | 0.275 | 1.209 | 1.289 |
| Functions | | | | | |
| NK Cell | 1.761 | 0.69 | −0.388 | 0.266 | −0.345 |
| Functions | | | | | |
| Pathogen | 1.903 | 0.654 | 0.93 | 0.306 | 0.672 |
| Response | | | | | |
| Senescence | 1.708 | 0.933 | 0.881 | 0.725 | 0.839 |
| T-Cell | 1.807 | 0.517 | 0.292 | 0.184 | −0.111 |
| Functions | | | | | |
| TLR | 1.927 | 0.7 | −0.482 | 0.324 | −0.207 |
| TNF | 0.694 | −0.41 | −0.725 | −0.734 | −0.715 |
| Superfamily | | | | | |
| Transporter | 1.697 | 0.491 | −0.385 | 0.524 | −0.331 |
| Functions | | | | | |

TABLE 7
Directed Global Significance scores for gene set
enrichment analysis when Group 6 is used as baseline
for comparison (Left Tumours)
LEFT TUMOUR

| | DIRECTED GLOBAL SIGNIFICANCE SCORE | | | | |
|---|---|---|---|---|---|
| | Gr1 vs. | Gr2 vs. | Gr3 vs. | Gr4 vs. | Gr5 vs. |
| | baseline | baseline | baseline | baseline | baseline |
| | of Gr6 | of Gr6 | of Gr6 | of Gr6 | of Gr6 |
| Adaptive | 1.271 | −0.999 | −0.845 | −1.054 | −1.094 |
| Adhesion | 0.635 | −0.86 | −0.509 | −0.905 | −0.89 |
| Antigen | 2.231 | 0.608 | 0.641 | 1.134 | 0.544 |
| Processing | | | | | |
| Apoptosis | 0.626 | −0.849 | −0.297 | −0.853 | −0.55 |
| B-Cell | 0.946 | −0.791 | −0.545 | −0.676 | −0.752 |
| Functions | | | | | |
| Basic Cell | 1.53 | −1.013 | −0.753 | −0.99 | −0.905 |
| Functions | | | | | |
| Cancer | 0.965 | −1.279 | −1.217 | −1.168 | −1.07 |
| Progression | | | | | |
| CD molecules | 1.274 | −0.728 | −0.58 | −0.658 | −0.758 |
| Cell Cycle | −1.031 | −1.045 | 0.319 | −1.004 | −0.066 |
| Chemokines & | 1.246 | −1.229 | −1.141 | −1.216 | −1.319 |
| Receptors | | | | | |
| Complement | 0.989 | −0.852 | −1.21 | −0.731 | −1.19 |
| Pathway | | | | | |
| Cytokines & | 0.958 | −1.085 | −0.766 | −0.962 | −1.027 |
| Receptors | | | | | |
| Dendritic Cell | 2.12 | −0.443 | −0.533 | 0.368 | −1.003 |
| Functions | | | | | |
| Humoral | 1.071 | −1.058 | −0.827 | −0.907 | −1.067 |
| Inflammation | 1.081 | −1.276 | −0.974 | −1.24 | −1.188 |
| Innate | 1.45 | −1.026 | −0.572 | −0.812 | −0.869 |
| Interferon | 2.78 | 0.938 | 0.833 | 1.162 | 1.129 |
| Interleukins | 1.101 | −0.86 | −0.249 | −0.713 | −0.784 |
| Leukocyte | 1.146 | −0.998 | −0.9 | −1.006 | −1.115 |
| Functions | | | | | |
| Macrophage | 1.24 | −1.161 | −0.894 | −0.912 | −0.741 |
| Functions | | | | | |
| MHC | 2.331 | 0.662 | 0.579 | 1.173 | 0.671 |
| Microglial | 2.894 | 1.24 | 1.122 | 1.36 | 0.986 |
| Functions | | | | | |
| NK Cell | 1.581 | −0.897 | 0.188 | −0.521 | −0.712 |
| Functions | | | | | |
| Pathogen | 1.012 | −1.583 | −0.624 | −1.445 | −1.347 |
| Response | | | | | |
| Senescence | −0.842 | −1.556 | −0.821 | −1.314 | −1.127 |
| T-Cell | 1.383 | −0.932 | −0.569 | −0.741 | −0.804 |
| Functions | | | | | |
| TLR | 1.143 | −0.938 | −0.446 | −0.933 | −0.765 |
| TNF | 0.337 | −0.679 | −0.449 | −0.628 | −0.618 |
| Superfamily | | | | | |
| Transporter | 1.442 | −1.085 | −0.777 | −0.781 | −0.741 |
| Functions | | | | | |

TABLE 8
Directed Global Significance scores for gene set enrichment
analysis when Group 1 is used as baseline for comparison

| | DIRECTED GLOBAL SIGNIFICANCE SCORE | | | | |
|---|---|---|---|---|---|
| | Gr2 vs. | Gr3 vs. | Gr4 vs. | Gr5 vs. | Gr6 vs. |
| | baseline | baseline | baseline | baseline | baseline |
| Pathway | of Gr1 | of Gr1 | of Gr1 | of Gr1 | of Gr1 |

RIGHT TUMOUR

| | | | | | |
|---|---|---|---|---|---|
| Adaptive | −1.357 | −1.697 | −1.293 | −1.721 | −1.548 |
| Adhesion | −1.047 | −0.783 | −0.936 | −0.39 | −1.076 |
| Antigen | −2.022 | −2.483 | −1.676 | −2.329 | −2.559 |
| Processing | | | | | |
| Apoptosis | −1.104 | −1.191 | −1.198 | −1.014 | −1.464 |
| B-Cell Functions | −1.191 | −1.391 | −1.257 | −1.48 | −1.267 |
| Basic Cell | −1.284 | −1.943 | −1.394 | −1.701 | −1.992 |
| Functions | | | | | |
| Cancer | −1.122 | −1.447 | −1.097 | −1.264 | −1.431 |
| Progression | | | | | |
| CD molecules | −1.335 | −1.533 | −1.304 | −1.44 | −1.543 |
| Cell Cycle | 0.751 | 0.516 | −0.79 | 0.849 | −1.014 |
| Chemokines & | −1.342 | −1.699 | −1.272 | −1.608 | −1.514 |
| Receptors | | | | | |
| Complement | −1.209 | −1.832 | −1.42 | −1.639 | −1.367 |
| Pathway | | | | | |
| Cytokines & | −1.166 | −1.401 | −1.203 | −1.426 | −1.247 |
| Receptors | | | | | |
| Dendritic Cell | −2.012 | −2.469 | −1.627 | −2.403 | −2.057 |
| Functions | | | | | |
| Humoral | −1.202 | −1.387 | −1.161 | −1.529 | −1.347 |
| Inflammation | −1.237 | −1.574 | −1.319 | −1.505 | −1.306 |
| Innate | −1.503 | −1.784 | −1.507 | −1.667 | −1.934 |
| Interferon | −2.245 | −2.924 | −2.035 | −2.76 | −3.29 |
| Interleukins | −1.226 | −1.398 | −1.296 | −1.471 | −1.44 |
| Leukocyte | −1.695 | −1.642 | −1.611 | −1.701 | −1.775 |
| Functions | | | | | |
| Macrophage | −1.157 | −1.546 | −1.003 | −1.561 | −1.256 |
| Functions | | | | | |
| MHC | −2.05 | −2.565 | −1.755 | −2.373 | −2.63 |
| Microglial | −2.105 | −2.418 | −2.102 | −2.261 | −3.057 |
| Functions | | | | | |
| NK Cell | −1.159 | −1.577 | −1.24 | −1.508 | −1.761 |
| Functions | | | | | |
| Pathogen | −1.445 | −1.597 | −1.636 | −1.624 | −1.903 |
| Response | | | | | |
| Senescence | −0.846 | −1.268 | −1.112 | −0.811 | −1.708 |
| T-Cell Functions | −1.43 | −1.67 | −1.474 | −1.683 | −1.808 |
| TLR | −1.455 | −1.937 | −1.655 | −1.876 | −1.927 |
| TNF Superfamily | −0.684 | −1.08 | −0.942 | −0.926 | −0.694 |
| Transporter | −1.43 | −1.618 | −1.31 | −1.529 | −1.697 |
| Functions | | | | | |

LEFT TUMOUR

| | | | | | |
|---|---|---|---|---|---|
| Adaptive | −1.414 | −1.482 | −1.395 | −1.608 | −1.271 |
| Adhesion | −0.976 | −0.555 | −0.963 | −0.911 | −0.635 |
| Antigen | −1.733 | −1.801 | −1.308 | −1.838 | −2.231 |
| Processing | | | | | |
| Apoptosis | −1.083 | −0.753 | −1.107 | −0.845 | −0.626 |
| B-Cell Functions | −1.18 | −1.183 | −1.058 | −1.229 | −0.946 |
| Basic Cell | −1.547 | −1.521 | −1.588 | −1.526 | −1.53 |
| Functions | | | | | |
| Cancer | −1.337 | −1.565 | −1.333 | −1.337 | −0.965 |
| Progression | | | | | |
| CD molecules | −1.321 | −1.355 | −1.236 | −1.448 | −1.274 |
| Cell Cycle | −0.537 | 0.864 | −0.595 | 0.758 | 1.031 |
| Chemokines & | −1.395 | −1.572 | −1.393 | −1.525 | −1.246 |
| Receptors | | | | | |
| Complement | −1.213 | −1.542 | −1.046 | −1.562 | −0.989 |
| Pathway | | | | | |
| Cytokines & | −1.265 | −1.208 | −1.173 | −1.365 | −0.958 |
| Receptors | | | | | |
| Dendritic Cell | −1.673 | −2.177 | −1.631 | −2.073 | −2.12 |
| Functions | | | | | |
| Humoral | −1.32 | −1.334 | −1.152 | −1.416 | −1.071 |
| Inflammation | −1.388 | −1.366 | −1.36 | −1.472 | −1.081 |
| Innate | −1.484 | −1.401 | −1.4 | −1.534 | −1.45 |
| Interferon | −1.932 | −2.241 | −1.782 | −1.846 | −2.78 |
| Interleukins | −1.273 | −1.095 | −1.196 | −1.408 | −1.101 |
| Leukocyte | −1.458 | −1.493 | −1.387 | −1.759 | −1.146 |
| Functions | | | | | |
| Macrophage | −1.344 | −1.513 | −1.244 | −1.323 | −1.24 |
| Functions | | | | | |
| MHC | −1.81 | −1.961 | −1.403 | −1.876 | −2.331 |
| Microglial | −2.303 | −1.858 | −1.722 | −2.088 | −2.894 |
| Functions | | | | | |
| NK Cell | −1.487 | −1.471 | −1.237 | −1.518 | −1.581 |
| Functions | | | | | |
| Pathogen | −1.525 | −0.674 | −1.456 | −1.391 | −1.012 |
| Response | | | | | |
| Senescence | −1.197 | −0.669 | −1.045 | −0.687 | 0.842 |
| T-Cell Functions | −1.462 | −1.389 | −1.284 | −1.497 | −1.383 |
| TLR | −1.312 | −1.219 | −1.243 | −1.216 | −1.143 |
| TNF Superfamily | −0.895 | −0.631 | −0.773 | −0.632 | −0.337 |
| Transporter | −1.565 | −1.577 | −1.451 | −1.604 | −1.442 |
| Functions | | | | | |

1. A method of treating cancer in a mammal, said method comprising the steps of:
(i) administering to the mammal a first composition comprising a protein antigen and/or a nucleic acid encoding the antigen and
(ii) administering to the mammal a second composition comprising an oncolytic virus wherein the oncolytic virus comprises a nucleic acid encoding the antigen.

2. (canceled)

3. The method according to claim 1 wherein the first composition comprises a protein antigen.

4. The method according to claim 1 wherein the first composition comprises a nucleic acid encoding the antigen.

5. The method according to claim 1 wherein the first and the second compositions are administered sequentially.

6. The method according to claim 5 wherein the first composition is administered first, followed by the second composition.

7. The method according to claim 1 wherein the first composition comprises a protein antigen and a nucleic acid encoding the antigen, wherein the protein antigen and the nucleic acid are administered simultaneously or sequentially.

8. The method according to claim 1 wherein the cancer is a solid tumour.

9. The method according to claim 8, wherein the first composition and/or the second composition is administered intratumorally.

10. The method according to claim 1 wherein the nucleic acid in the first composition is comprised in a vector.

11. The method according to claim 10 wherein the vector is a viral vector.

12. The method according to claim 10 wherein the vector is a self-amplifying RNA molecule.

13. The method according to claim 1 wherein the oncolytic virus is Newcastle disease virus (NDV) or Modified Vaccinia Ankara (MVA).

14. The method according to claim 1 wherein the antigen is a non-self antigen.

15. The method according to claim 1 wherein administration of the first and/or the second composition leads to differential expression of genes within the tumour microenvironment involved in one or more gene sets that characterise a particular pathway of anti-cancer immunity.
